(19) United States
(12) Patent Application Publication
(10) Pub. No.: US 2026/0035346 A1
(43) Pub. Date: Feb. 5, 2026

(54) CO-CRYSTALS OF 2-BROMO-1-(3,3-DINITROAZETIDIN-1-YL)ETHANONE AND METHODS

(71) Applicant: EpicentRx, Inc., La Jolla, CA (US)

(72) Inventors: James KANTER, La Jolla, CA (US); Bryan T. ORONSKY, Los Altos Hills, CA (US); Scott CAROEN, San Francisco, CA (US); Tony R. REID, San Diego, CA (US)

(73) Assignee: EpicentRx, Inc., La Jolla, CA (US)

(21) Appl. No.: 18/847,878

(22) PCT Filed: Mar. 16, 2023
(86) PCT No.: PCT/US2023/064591

§ 371 Date: Sep. 17, 2024

Related U.S. Application Data
(60) Provisional application No. 63/321,592, filed on Mar. 18, 2022.

(51) Int. Cl. C07D 205/04; A61K 31/397; A61K 35/18; A61K 45/06; C07D 213/67; C07D 417/06

(52) U.S. Cl. CPC C07D 205/04; A61K 31/397; A61K 35/18; A61K 45/06; C07D 213/67; C07D 417/06

(57) ABSTRACT

The present disclosure relates generally to crystals, and more specifically to co-crystals of RRx-001 or 2-bromo-1-(3,3-dinitroazetidin-1-yl)ethanone and methods of preparing and using the co-crystals to treat or prevent various diseases, disorders, and conditions.

### CROSS-REFERENCE TO RELATED APPLICATIONS SECTION

This application claims priority to U.S. Provisional Patent Application Ser. 63/321,592 filed on Mar. 18, 2022, the entire contents of which are hereby incorporated by reference in their entirety.

### FIELD OF THE INVENTION

The present disclosure relates generally to crystals, and more specifically to co-crystals of RRx-001 or 2-bromo-1-(3,3-dinitroazetidin-1-yl)ethanone and methods of preparing and using the co-crystals to prevent or treat various diseases, disorders, and conditions.

### BACKGROUND

Co-crystals have generated tremendous interest in pharmaceutical research and development because of the potential to customize physicochemical properties of the solid while maintaining the chemical integrity of the drug. Pharmaceutically relevant properties that can change via co-crystallization include but are not limited to solubility, dissolution, moisture uptake, chemical stability, mechanical properties, and bioavailability.

2-bromo-1-(3,3-dinitroazetidin-1-yl)ethanone, also referred to as Compound 1 herein, is currently being clinically evaluated for the treatment of various cancers as well as an antiradiation agent or radioprotectant to be used against nuclear radiation which may be encountered during military conflict or a nuclear meltdown and a radioprotectant and chemoprotectant intended to reduce the undesirable adverse effects of chemotherapy and radiation therapy during the treatment of cancer. Further, the compound is being considered as an effective blood preservative.

However, Compound 1 drug substance, despite many advantageous properties, is subject to detonation by application of shock or impact which creates potential safety issues when making, transporting, storing, and formulating the drug substance for use in therapy. Accordingly, there remains a need to create a consistently non-explosive form of Compound 1 to enable its safe storage, transport and general handling for use in therapeutic applications and as an anti-inflammatory or antioxidant agent for the treatment of autoimmune and inflammatory conditions. Furthermore, Compound 1 has relatively low solubility in water, and there remains a need to create more water soluble forms of the compound to improve administration and use.

### BRIEF SUMMARY

In one aspect, provided is a co-crystal comprising Compound 1 and calcium chloride. In some embodiments, Compound 1 calcium chloride co-crystal is characterized by having an XRPD pattern comprising peaks at angles 2-theta of 10.8±0.2, 11.6±0.2, and 14.9±0.2 degrees. In some embodiments, Compound 1 calcium chloride co-crystal is characterized by having an XRPD pattern comprising peaks at angles 2-theta of 10.8±0.2, 11.6±0.2, 12.9±0.2, and 14.9±0.2 degrees. In some embodiments, Compound 1 calcium chloride co-crystal is characterized by having an XRPD pattern comprising peaks at angles 2-theta of 10.8±0.2, 11.6±0.2, 12.9±0.2, 14.9±0.2, 17.7±0.2, 18.0±0.2, 18.2±0.2, 18.9±0.2, 19.2±0.2, 19.9±0.2, 20.7±0.2, 21.5±0.2, 21.9±0.2, 22.9±0.2, 25.0±0.2, 25.9±0.2, 26.7±0.2, 27.1±0.2, 27.7±0.2, 28.3±0.2, 29.0±0.2, 29.1±0.2, 29.9±0.2, 30.4±0.2, 31.0±0.2, 31.8±0.2, 32.2±0.2, 32.5±0.2, 32.9±0.2, 33.4±0.2, 33.6±0.2, 34.2±0.2, 34.7±0.2, 35.5±0.2, 35.6±0.2, 35.8±0.2, 36.7±0.2, 37.0±0.2, 37.5±0.2, 38.0±0.2, and 39.0±0.2 degrees. In some embodiments, Compound 1 calcium chloride co-crystal is characterized by having an XRPD pattern substantially as shown in FIG. 1A. In some embodiments, Compound 1 calcium chloride co-crystal is characterized by having a DSC graph comprising an endothermic peak at about 136.2° C. In some embodiments, Compound 1 calcium chloride co-crystal is characterized by having a DSC graph substantially as shown in FIG. 1B. In some embodiments, Compound 1 calcium chloride co-crystal is characterized by having a TGA graph substantially as shown in FIG. 1C. In some embodiments, Compound 1 calcium chloride co-crystal is characterized by having a DVS graph substantially as shown in FIG. 1D. In some embodiments, Compound 1 calcium chloride co-crystal is characterized by having an IR spectrum comprising peaks at wavenumbers of about 411±2, 450±2, 510±2, 551±2, 568±2, 654±2, 708±2, 724±2, 757±2, 790±2, 841±2, 871±2, 897±2, 926±2, 1009±2, 1097±2, 1116±2, 1191±2, 1214±2, 1253±2, 1285±2, 1308±2, 1339±2, 1369±2, 1414±2, 1438±2, 1462±2, 1567±2, 1586±2, 1644±2, 1679±2, 2956±2, 3005±2, 3020±2, and 3387±2 cm⁻¹. In some embodiments, Compound 1 calcium chloride co-crystal is characterized by having IR spectrum substantially as shown in FIG. 1F. In some embodiments. Compound 1 calcium chloride co-crystal is characterized by having an FT-Raman spectrum comprising peaks at wavenumbers of about 257±2, 413±2, 448±2, and 791±2 cm⁻¹. In some embodiments, Compound 1 calcium chloride co-crystal is characterized by having an FT-Raman spectrum comprising peaks at wavenumbers of about 122±2, 161±2, 190±2, 257±2, 282±2, 309±2, 340±2, 413±2, 448±2, 512±2, 570±2, 656±2, 710±2, 724±2, 757±2, 791±2, 841±2, 870±2, 897±2, 1009±2, 1098±2, 1123±2, 1189±2, 1214±2, 1252±2, 1273±2, 1287±2, 1308±2, 1341±2, 1370±2, 1414±2, 1461±2, 1569±2, 1590±2, 1680±2, 2866±2, 2961±2, 2999±2, and 3019±2 cm⁻¹. In some embodiments, Compound 1 calcium chloride co-crystal is characterized by having an FT-Raman spectrum substantially as shown in FIG. 1G. In some embodiments, Compound 1 calcium chloride co-crystal is characterized by having an LF-Raman spectrum comprising peaks at wavenumbers of about −340±2, −309±2, −282±2, −256±2, −189±2, −121±2, −76±2, −46±2, and −35±2 cm⁻¹. In some embodiments, Compound 1 calcium chloride co-crystal is characterized by having an LF-Raman spectrum substantially as shown in FIG. 1H. In some embodiments, Compound 1 calcium chloride co-crystal is characterized by having an ¹H NMR spectrum comprising peaks at chemical shifts of about 3.35, 4.28, 4.83, and 5.15 ppm. In some embodiments, Compound 1 calcium chloride co-crystal is characterized by having an ¹H NMR spectrum substantially as shown in FIG. 1I.

In some aspect, provided is a co-crystal comprising Compound 1 and pyridoxine HCl. In some embodiments, Compound 1 pyridoxine HCl co-crystal is characterized by having an XRPD pattern comprising peaks at angles 2-theta of 11.4±0.2, 12.7±0.2, and 14.8±0.2 degrees. In some embodiments, Compound 1 pyridoxine HCl co-crystal is characterized by having an XRPD pattern comprising peaks at angles 2-theta of 10.7±0.2, 11.4±0.2, 12.7±0.2, and 14.8±0.2 degrees. In some embodiments, Compound 1 pyridoxine HCl co-crystal is characterized by having an XRPD pattern comprising peaks at angles 2-theta of 10.3±0.2, 10.7±0.2, 10.9±0.2, 11.4±0.2, 12.7±0.2, 14.8±0.2, 15.6±0.2, 17.0±0.2, 17.5±0.2, 17.9±0.2, 18.7±0.2, 19.7±0.2, 20.0±0.2, 20.3±0.2, 20.8±0.2, 21.3±0.2, 21.6±0.2, 21.8±0.2, 22.6±0.2, 22.8±0.2, 23.3±0.2, 24.2±0.2, 24.8±0.2, 25.0±0.2, 25.8±0.2, 26.5±0.2, 26.9±0.2, 27.5±0.2, 27.8±0.2, 28.2±0.2, 28.7±0.2, 29.8±0.2, 30.2±0.2, 30.8±0.2, 31.5±0.2, 32.0±0.2, 32.2±0.2, 32.9±0.2, 33.8±0.2, 34.4±0.2, 35.3±0.2, 35.5±0.2, 37.0±0.2, 37.7±0.2, 38.1±0.2, and 38.8±0.2 degrees. In some embodiments, Compound 1 pyridoxine HCl co-crystal is characterized by having an XRPD pattern substantially as shown in FIG. 2A. In some embodiments, Compound 1 pyridoxine HCl co-crystal is characterized by having a DSC graph comprising an endothermic peak at about 134.7° C. In some embodiments, Compound 1 pyridoxine HCl co-crystal is characterized by having a DSC graph substantially as shown in FIG. 2B. In some embodiments, Compound 1 pyridoxine HCl co-crystal is characterized by having a TGA graph substantially as shown in FIG. 2C. In some embodiments, Compound 1 pyridoxine HCl co-crystal is characterized by having a DVS graph substantially as shown in FIG. 2D. In some embodiments, Compound 1 pyridoxine HCl co-crystal is characterized by having an IR spectrum comprising peaks at wavenumbers of about 409±2, 450±2, 475±2, 511±2, 575±2, 589±2, 622±2, 653±2, 685±2, 724±2, 749±2, 791±2, 841±2, 870±2, 926±2, 962±2, 989±2, 1017±2, 1089±2, 1115±2, 1191±2, 1214±2, 1253±2, 1275±2, 1307±2, 1339±2, 1368±2, 1382±2, 1400±2, 1413±2, 1439±2, 1447±2, 1460±2, 1480±2, 1545±2, 1567±2, 1587±2, 1625±2, 1645±2, 1679±2, 1732±2, 1805±2, 1826±2, 1900±2, 1929±2, 2817±2, 2955±2, 3093±2, 3232±2, 3267±2, and 3317±2 cm⁻¹. In some embodiments, Compound 1 pyridoxine HCl co-crystal is characterized by having an FT-Raman spectrum comprising peaks at wavenumbers of about 116±2, 161±2, 257±2, 286±2, 309±2, 329±2, 356±2, 404±2, 477±2, 517±2, 533±2, 568±2, 654±2, 691±2, 724±2, 753±2, 791±2, 841±2, 868±2, 897±2, 928±2, 965±2, 988±2, 1009±2, 1052±2, 1088±2, 1123±2, 1216±2, 1233±2, 1293±2, 1322±2, 1356±2, 1356±2, 1370±2, 1381±2, 1449±2, 1569±2, 1588±2, 1628±2, 1644±2, 1678±2, 2826±2, 2868±2, 2901±2, 2934±2, 2959±2, 2999±2, 3015±2, and 3102±2 cm⁻¹. In some embodiments, Compound 1 pyridoxine HCl co-crystal is characterized by having IR spectrum substantially as shown in FIG. 2F. In some embodiments, Compound 1 pyridoxine HCl co-crystal is characterized by having an FT-Raman spectrum comprising peaks at wavenumbers of about 477±2, 791±2, 1233±2, 1628±2, and 1644±2. In some embodiments, Compound 1 pyridoxine HCl co-crystal is characterized by having an FT-Raman spectrum substantially as shown in FIG. 2G. In some embodiments, Compound 1 pyridoxine HCl co-crystal is characterized by having an LF-Raman spectrum comprising peaks at wavenumbers of about −329±2, −285±2, −167±2, −118±2, −83±2, and −57±2 cm⁻¹. In some embodiments, Compound 1 pyridoxine HCl co-crystal is characterized by having an LF-Raman spectrum substantially as shown in FIG. 2H. In some embodiments, Compound 1 pyridoxine HCl co-crystal is characterized by having an ¹H NMR spectrum comprising peaks at chemical shifts of about 2.56, 4.07, 4.29, 4.71, 4.80, 4.84, 5.16, or 8.16 ppm. In some embodiments, Compound 1 pyridoxine HCl co-crystal is characterized by having an ¹H NMR spectrum substantially as shown in FIG. 2I.

In some aspect, provided is a co-crystal comprising Compound 1 and thiamine HCl. In some embodiments, Compound 1 thiamine HCl co-crystal is characterized by having an XRPD pattern comprising peaks at angles 2-theta of 8.2±0.2, 10.3±0.2, and 16.4±0.2 degrees. In some embodiments, Compound 1 thiamine HCl co-crystal is characterized by having an XRPD pattern comprising peaks at angles 2-theta of 7.5±0.2, 8.2±0.2, 10.3±0.2, 14.8±0.2, and 16.4±0.2 degrees. In some embodiments, Compound 1 thiamine HCl co-crystal is characterized by having an XRPD pattern comprising peaks at angles 2-theta of 7.5±0.2, 8.2±0.2, 10.3±0.2, 10.7±0.2, 11.9±0.2, 12.7±0.2, 14.8±0.2, 15.0±0.2, 16.4±0.2, 17.2±0.2, 17.5±0.2, 17.9±0.2, 18.2±0.2, 18.9±0.2, 19.3±0.2, 19.7±0.2, 20.6±0.2, 21.3±0.2, 21.6±0.2, 22.7±0.2, 23.0±0.2, 23.5±0.2, 23.6±0.2, 24.1±0.2, 24.7±0.2, 25.0±0.2, 25.3±0.2, 25.6±0.2, 25.9±0.2, 26.7±0.2, 27.5±0.2, 27.7±0.2, 28.1±0.2, 28.8±0.2, 29.2±0.2, 29.7±0.2, 30.1±0.2, 30.8±0.2, 31.1±0.2, 31.8±0.2, 32.1±0.2, 32.5±0.2, 32.7±0.2, 33.2±0.2, 33.7±0.2, 34.3±0.2, 34.8±0.2, 35.4±0.2, 35.7±0.2, 36.1±0.2, 36.4±0.2, 37.2±0.2, 37.7±0.2, 38.8±0.2, and 39.2±0.2 degrees. In some embodiments, Compound 1 thiamine HCl co-crystal is characterized by having an XRPD pattern substantially as shown in FIG. 3A. In some embodiments, Compound 1 thiamine HCl co-crystal is characterized by having a DSC graph comprising an endothermic peak at about 131.8° C. In some embodiments, Compound 1 thiamine HCl co-crystal is characterized by having a DSC graph substantially as shown in FIG. 3B. In some embodiments, Compound 1 thiamine HCl co-crystal is characterized by having a TGA graph substantially as shown in FIG. 3C. In some embodiments, Compound 1 thiamine HCl co-crystal is characterized by having a DVS graph substantially as shown in FIG. 3D. In some embodiments, Compound 1 thiamine HCl co-crystal is characterized by having an IR spectrum comprising peaks at wavenumbers of about 408±2, 440±2, 450±2, 463±2, 509±2, 542±2, 572±2, 645±2, 702±2, 724±2, 751±2, 760±2, 790±2, 841±2, 870±2, 891±2, 972±2, 1007±2, 1026±2, 1045±2, 1072±2, 1095±2, 1115±2, 1170±2, 1189±2, 1213±2, 1225±2, 1233±2, 1251±2, 1286±2, 1308±2, 1339±2, 1368±2, 1380±2, 1399±2, 1438±2, 1480±2, 1508±2, 1532±2, 1567±2, 1587±2, 1613±2, 1655±2, 2915±2, 2964±2, 3038±2, 3193±2, 3414±2, and 3498±2 cm⁻¹. In some embodiments, Compound 1 thiamine HCl co-crystal is characterized by having an FT-Raman spectrum comprising peaks at wavenumbers of about 109±2, 246±2, 282±2, 309±2, 346±2, 409±2, 517±2, 543±2, 571±2, 583±2, 627±2, 645±2, 666±2, 701±2, 751±2, 791±2, 820±2, 841±2, 868±2, 897±2, 932±2, 946±2, 973±2, 1007±2, 1057±2, 1189±2, 1212±2, 1235±2, 1250±2, 1283±2, 1308±2, 1331±2, 1370±2, 1381±2, 1449±2, 1480±2, 1507±2, 1569±2, 1590±2, 1613±2, 1651±2, 1678±2, 2882±2, 2932±2, 2959±2, 2998±2, and 3015±2 cm⁻¹. In some embodiments, Compound 1 thiamine HCl co-crystal is characterized by having IR spectrum substantially as shown in FIG. 3F. In some embodiments, Compound 1 thiamine HCl co-crystal is characterized by having an FT-Raman spectrum comprising peaks at wavenumbers of about 627±2, 751±2, 791±2, 820±2, 1480±2, 1651±2, and 2932±2 cm⁻¹. In some embodiments, Compound 1 thiamine HCl co-crystal is characterized by having an FT-Raman spectrum substantially as shown in FIG. 3G. In some embodiments, Compound 1 thiamine HCl co-crystal is characterized by having an LF-Raman spectrum comprising peaks at wavenumbers of about −346±2, −107±2, −77±2, −62±2, 40±2, and −29±2 cm⁻¹. In some embodiments, Compound 1 thiamine HCl co-crystal is characterized by having an LF-Raman spectrum substantially as shown in FIG. 3H. In some embodiments, Compound 1 thiamine HCl co-crystal is characterized by having an ¹H NMR spectrum comprising peaks at chemical shifts of about 2.56, 3.07, 3.08, 3.10, 3.66, 3.67, 3.68, 4.07, 4.29, 4.84, 5.16, and 5.50 ppm. In some embodiments, Compound 1 thiamine HCl co-crystal is characterized by having an ¹H NMR spectrum substantially as shown in FIG. 3I.

In some aspect, provided is a co-crystal comprising Compound 1 and ferric chloride. In some embodiments, Compound 1 ferric chloride co-crystal is characterized by having an XRPD pattern comprising peaks at angles 2-theta of 7.8±0.2, 13.5±0.2, and 15.7±0.2 degrees. In some embodiments, Compound 1 ferric chloride co-crystal is characterized by having an XRPD pattern comprising peaks at angles 2-theta of 7.8±0.2, 11.2±0.2, 13.5±0.2, 15.7±0.2, 16.0±0.2, and 16.2±0.2 degrees. In some embodiments. Compound 1 ferric chloride co-crystal is characterized by having an XRPD pattern comprising peaks at angles 2-theta of 7.8±0.2, 11.2±0.2, 13.5±0.2, 15.7±0.2, 16.0±0.2, 16.2±0.2, 17.5±0.2, 17.9±0.2, 20.6±0.2, 21.0±0.2, 22.1±0.2, 22.4±0.2, 24.2±0.2, 24.8±0.2, 26.2±0.2, 27.8±0.2, 28.3±0.2, 29.4±0.2, 31.1±0.2, 31.4±0.2, 31.6±0.2, 32.0±0.2, 32.4±0.2, 32.6±0.2, 33.4±0.2, 33.9±0.2, 34.2±0.2, 35.2±0.2, 36.1-0.2, 37.6±0.2, 38.0±0.2, 38.7±0.2, and 39.7±0.2 degrees. In some embodiments. Compound 1 ferric chloride co-crystal is characterized by having an XRPD pattern substantially as shown in FIG. 4A. In some embodiments, Compound 1 ferric chloride co-crystal is characterized by having a DSC graph comprising an endothermic peak at about 40° C. In some embodiments, Compound 1 ferric chloride co-crystal is characterized by having a DSC graph substantially as shown in FIG. 4B. In some embodiments, Compound 1 ferric chloride co-crystal is characterized by having a TGA graph substantially as shown in FIG. 4C. In some embodiments, Compound 1 ferric chloride co-crystal is characterized by having an ¹H NMR spectrum comprising peaks at chemical shifts of about 4.21, 4.77, and 5.09 ppm. In some embodiments, Compound 1 ferric chloride co-crystal is characterized by having an ¹H NMR spectrum substantially as shown in FIG. 4D.

In some aspect, provided is a co-crystal comprising Compound 1 and manganese chloride. In some embodiments, Compound 1 manganese chloride co-crystal is characterized by having an XRPD pattern comprising peaks at angles 2-theta of 12.7±0.2, 14.7±0.2, and 15.3±0.2 degrees. In some embodiments, Compound 1 manganese chloride co-crystal is characterized by having an XRPD pattern comprising peaks at angles 2-theta of 10.6±0.2, 11.4±0.2, 12.7±0.2, 14.7±0.2, 15.3-0.2, and 15.8±0.2 degrees. In some embodiments, Compound 1 manganese chloride co-crystal is characterized by having an XRPD pattern comprising peaks at angles 2-theta of 10.6±0.2, 11.4±0.2, 12.7±0.2, 14.7±0.2, 15.3±0.2, 15.8±0.2, 17.5±0.2, 17.8±0.2, 18.3±0.2, 19.7±0.2, 20.5±0.2, 21.2±0.2, 21.7±0.2, 22.7±0.2, 24.3±0.2, 24.7±0.2, 24.9±0.2, 25.4±0.2, 26.5±0.2, 26.8±0.2, 27.5±0.2, 28.1±0.2, 28.8±0.2, 29.7±0.2, 30.1±0.2, 30.9±0.2, 31.9±0.2, 32.2±0.2, 32.8±0.2, 33.2±0.2, 33.8±0.2, 34.4±0.2, 35.3±0.2, 35.5±0.2, 37.7±0.2, 38.8±0.2, and 39.6±0.2 degrees. In some embodiments, Compound 1 manganese chloride co-crystal is characterized by having an XRPD pattern substantially as shown in FIG. 5A. In some embodiments, Compound 1 manganese chloride co-crystal is characterized by having a DSC graph comprising endothermic peaks at about 58.8° C., 121.2° C., 130.6° C., and 195.5° C. In some embodiments, Compound 1 manganese chloride co-crystal is characterized by having a DSC graph substantially as shown in FIG. 5B. In some embodiments, Compound 1 manganese chloride co-crystal is characterized by having a TGA graph substantially as shown in FIG. 5C. In some embodiments, Compound 1 manganese chloride co-crystal is characterized by having an ¹H NMR spectrum comprising peaks at chemical shifts of about 4.05, 4.27, 4.82, and 5.14 ppm. In some embodiments, Compound 1 manganese chloride co-crystal is characterized by having an ¹H NMR spectrum substantially as shown in FIG. 5D.

In some aspect, provided is a co-crystal comprising Compound 1 and zinc chloride. In some embodiments, Compound 1 zinc chloride co-crystal is characterized by having an XRPD pattern comprising peaks at angles 2-theta of 13.4±0.2, 15.1±0.2, and 17.1±0.2 degrees. In some embodiments, Compound 1 zinc chloride co-crystal is characterized by having an XRPD pattern comprising peaks at angles 2-theta of 11.2±0.2, 13.4±0.2, 15.1±0.2, 16.5±0.2, and 17.1±0.2 degrees. In some embodiments, Compound 1 zinc chloride co-crystal is characterized by having an XRPD pattern comprising peaks at angles 2-theta of 11.2-0.2, 13.4±0.2, 15.1±0.2, 16.5±0.2, 17.1±0.2, 17.5±0.2, 18.3±0.2, 18.7±0.2, 19.3±0.2, 20.7±0.2, 21.4±0.2, 22.0±0.2, 22.1±0.2, 22.5±0.2, 22.8±0.2, 23.0±0.2, 23.4±0.2, 24.0±0.2, 24.9±0.2, 25.1±0.2, 26.9±0.2, 27.2±0.2, 28.1±0.2, 28.4±0.2, 29.0±0.2, 29.3±0.2, 29.6±0.2, 30.1±0.2, 30.5±0.2, 31.4±0.2, 31.7±0.2, 31.9±0.2, 32.4±0.2, 32.8±0.2, 33.3±0.2, 34.4±0.2, 35.1±0.2, 35.6±0.2, 36.0±0.2, 36.8±0.2, 37.0±0.2, 37.8±0.2, and 38.6±0.2 degrees. In some embodiments, Compound 1 zinc chloride co-crystal is characterized by having an XRPD pattern substantially as shown in FIG. 6A. In some embodiments, Compound 1 zinc chloride co-crystal is characterized by having a DSC graph comprising endothermic peaks at about 124.1° C. and 153° C. In some embodiments, Compound 1 zinc chloride co-crystal is characterized by having a DSC graph substantially as shown in FIG. 6B. In some embodiments, Compound 1 zinc chloride co-crystal is characterized by having a TGA graph substantially as shown in FIG. 6C. In some embodiments, Compound 1 zinc chloride co-crystal is characterized by having an ¹H NMR spectrum comprising peaks at chemical shifts of about 4.06, 4.84, and 5.15 ppm. In some embodiments, Compound 1 zinc chloride co-crystal is characterized by having an ¹H NMR spectrum substantially as shown in FIG. 6D.

In some aspect, provided are compositions comprising one or more co-crystals described herein, and a pharmaceutically acceptable excipient.

In some aspect, provided are methods of treating or preventing a cancer. In some embodiments, the method comprises intravenously or orally administering to a mammal in need thereof an effective amount of the co-crystal or the composition described herein.

In some aspect, provided are methods of treating or preventing an ischemic or hypoxic condition. In some embodiments, the method comprises administering to a mammal in need thereof an effective amount of the co-crystal or the composition described herein.

Another embodiment of the present invention provides a pharmaceutical composition comprising a co-crystal and a pharmaceutically acceptable excipient. In some embodiments, the pharmaceutical composition further comprises a blood product. In some embodiments, the blood product comprises erythrocyte cells. In some embodiments, the erythrocyte cells have not undergone any manipulation selected from the group consisting of genetic modification, electroporation, conjugation through biotin, conjugation to a cell-penetrating peptide, conjugation to hemoglobin, dimethyl sulfoxide osmotic pulse, endocytosis and hypotonic preswelling, hypotonic dilution, and hypo-osmotic dialysis. In some embodiments, the blood product is a mixture of packed red blood cells. In some embodiments, the blood product is whole blood. In some embodiments, the whole blood is autologous whole blood or donor-matched allogenic whole blood.

In some embodiments, the pharmaceutical composition comprises the blood product and/or at least one agent. In some embodiments, each agent of the at least one agent is selected from the group consisting of: a sympathomimetic drug, an anti-inflammatory agent, an anti-diabetic agent, an anti-fibrotic agent, an anti-steatotic agent, a cholesterol/lipid modulating agent, an anti-cancer agent, and an anti-diabetic agent. In some embodiments, each agent of the at least one agent is selected from the group consisting of: an antioxidant, a vitamin, a mineral, a steroid, a growth hormone, a nitric oxide donor, a terpene-indole alkaloid compound, a bisphosphonate compound, a glucocorticoid, a coagulation factor, a narcotic, an opioid receptor agonist, a narcotic receptor antagonist, an anti-viral agent, a biologic response modifier (BRM) agent, and an antigen.

A further embodiment of the present invention provides a method for preventing or treating a disease, a disorder, or a condition. The method comprises administering an effective amount of the co-crystal or the pharmaceutical composition to a subject in need thereof. In some embodiments, the subject is a mammal subject. In some embodiments, the mammal subject is a human subject. In other embodiments, the mammal subject is a non-human subject. In some embodiments, the effective amount of the co-crystal or the pharmaceutical composition is a therapeutically effective amount of the co-crystal or the pharmaceutical composition.

In some embodiments, the disease is associated with abnormal cell proliferation. In some embodiments, the disease is selected from the group consisting of: a neurodegenerative disease, an allergic disease, an autoimmune disease, a fibrotic disease, an inflammatory disease, an infectious disease, a pulmonary disease, a reproductive-related disease, a cardiovascular disease, and a metabolic disease.

In some embodiments, the condition comprises an ischemic or hypoxic condition. In some embodiments, the ischemic condition comprises an acute ischemic condition or a chronic ischemic condition. In some embodiments, ischemic condition comprises the acute ischemic condition and is selected from the group consisting of: myocardial infarction, ischemic stroke, pulmonary embolism, perinatal hypoxia, circulatory shock, mountain sickness, and acute respiratory failure. In some embodiments, the ischemic condition comprises the chronic ischemic condition and is selected from the group consisting of: atherosclerosis, chronic venous insufficiency, chronic heart failure, cardiac cirrhosis, diabetes, macular degeneration, sleep apnea, Raynaud's disease, systemic sclerosis, nonbacterial thrombotic endocarditis, occlusive artery disease, angina pectoris, transient ischemic attacks, and chronic alcoholic liver disease.

In some embodiments, the condition comprises a hypoxic condition. In some embodiments, the hypoxic condition is selected from the group consisting of: cancer, gastric or duodenal ulcers, liver or renal disease, thrombocytopenia, a blood coagulation disorder, and a chronic illness.

In some embodiments, the condition comprises a hemolytic condition. The hemolytic condition is selected from the group consisting of: sickle cell crisis, thalassemia, hemoglobin C disease, hemoglobin SC disease, sickle thalassemia, hereditary spherocytosis, hereditary elliptocytosis, hereditary ovalocytosis, glucose-6-phosphate deficiency, paroxysmal nocturnal hemoglobinuria (PNH), paroxysmal cold hemoglobinuria (PCH), thrombotic thrombocytopenic purpura/hemolytic uremic syndrome (TTP/HUS), idiopathic autoimmune hemolytic anemia, drug-induced immune hemolytic anemia, secondary immune hemolytic anemia, non-immune hemolytic anemia, malaria, falciparum malaria, bartonellosis, babesiosis, clostridial infection, severe Haemophilus influenzae type b infection, extensive burns, transfusion reaction, rhabdomyolysis (myoglobinemia), cardiopulmonary bypass, and hemodialysis.

In some embodiments, the disease, the disorder, or the condition is selected from the group consisting of: pulmonary hypertension, hyperlipidemia, muscular dystrophies, peripheral vascular disease, patent foramen ovale, obesity, heart failure, mitochondrial disorders or diseases, chronic obstructive pulmonary disease, hyperCKemia, motor neuron disease, neuromuscular disease, multiple sclerosis, Charcot-myositis including polymyositis and dermatomyositis, insulin resistance, myoedema, rhabdomyolysis, idiopathic chronic muscle fatigue, reduced skeletal muscle function, disrupted skeletal muscle function or metabolism, cardiac abnormalities, non-alcoholic fatty liver disease (NAFLD), non-alcoholic steatohepatitis (NASH), hypercholesterolemia, steatosis, liver fibrosis, coronary heart disease, peripheral vascular disease, aortic aneurysm, and glucose intolerance.

In some embodiments, the disease is cancer. In some embodiments, the cancer is a solid tumor. In some embodiments, the cancer is selected from the group consisting of: brain cancer, bladder cancer, breast cancer, cervical cancer, cholangiocarcinoma, colon cancer, colorectal cancer, endometrial cancer, esophageal cancer, lung cancer, liver cancer, melanoma, ovarian cancer, pancreatic cancer, prostate cancer, rectal cancer, renal cancer, stomach cancer, testicular cancer, uterine cancer, leukemia, and lymphoma. In some embodiments, the cancer is lymphoma. In some embodiments, the lymphoma is selected from the group consisting of: a B-cell lymphoma and non-Hodgkin lymphoma.

A further embodiment of the present invention describes a method for preventing or treating an infection. The method includes administering an effective amount of the co-crystal or the pharmaceutical composition to the subject in need thereof. In some embodiments, the subject is a mammal subject. In some embodiments, the mammal subject is a human subject. In some embodiments, the mammal subject is a non-human subject. In some embodiments, the effective amount of the co-crystal or the pharmaceutical composition is a therapeutically effective amount of the co-crystal or the pharmaceutical composition.

In some embodiments, the infection comprises a bacterial infection. In some embodiments, the bacterial infection comprises a gram-positive bacterial infection. In some embodiments, the gram-positive bacterial infection comprises a gram-positive cocci bacterial infection or a gram-positive bacilli bacterial infection. In some embodiments, the bacterial infection comprises a gram-negative bacterial infection. In some embodiments, the gram-negative bacterial infection comprises a gram-negative cocci bacterial infection or a gram-negative bacilli bacterial infection.

In some embodiments, the bacterial infection is an anaerobic bacterial infection. In some embodiments, the bacterial infection is an aerobic bacterial infection. In some embodiments, the bacterial infection is a mycobacterial infection. In some embodiments, the bacterial infection is caused by a bacteria selected from the group consisting of: Mycobacterium tuberculosis, Staphylococcus aureus, Staphylococcus epidermidis, Enterococcus faecalis, Enterococcus faecium, Streptococcus pneumoniae, Streptococcus pyogenes, Mycobacterium smegmatis, Bacillus anthracis, Escherichia coli, Proteus mirabilis, Pseudomonas aeruginosa, Acinetobacter baumannii. Yersinia enterocolitica, Francisella tularensis, Eubacterium lentum, Bacteroides fragilis, Fusobacterium nucleatum, Porphyromonas asaccharolyticus, Clostridium perfringens, Mycobacterium tuberculosis, and Clostridium difficile. In some embodiments, the bacterial infection is caused by a bacteria having a genus selected from the group consisting of: Peptostreptococci, Actinomyces, Clostridium, Anaerobiospirillum, Bacteroides, Prevotella, Fusobacterium, and Bilophila. In some embodiments, the bacterial infection occurs from an antibiotic-resistant bacteria.

In some aspect, provided are methods of protecting against normal tissue toxicity caused by chemotherapy and/or radiation therapy. In some embodiments, the method comprises subcutaneously administering to a mammal in need thereof an effective amount of the co-crystal or the composition described herein before the mammal is exposed to the chemotherapy and/or radiation therapy.

In some aspect, provided are methods of treating or preventing neurodegenerative, allergic, autoimmune, fibrotic, inflammatory, infectious, pulmonary, cardiac, vascular, or metabolic diseases. In some embodiments, the method comprises subcutaneously administering to a mammal in need thereof an effective amount of the co-crystal or the composition described herein.

In some aspect, provided are methods of treating a mammal suffering from reduced blood volume or low perfusion. In some embodiments, the method comprises administering to the mammal in need thereof a blood product comprising the co-crystal described herein.

In some aspect, provided are methods of preparing Compound 1 calcium chloride co-crystal. In some embodiments, the method comprises a) mixing molar equivalents of Compound 1 and calcium chloride. In some embodiments, the method comprises b) mixing ethanol and water with Compound 1 and calcium chloride to form a slurry. In some embodiments, the method comprises c) stirring the slurry of step b) to obtain stirred slurry. In some embodiments, the method comprises d) filtering the stirred slurry of step c) to obtain solid content. In some embodiments, the method comprises e) drying the solid content of step d).

In some aspect, provided are methods of preparing Compound 1 pyridoxine HCl co-crystal. In some embodiments, the method comprises a) mixing molar equivalents of Compound 1 and pyridoxine HCl. In some embodiments, the method comprises b) mixing ethanol and water with Compound 1 and pyridoxine HCl to form a slurry. In some embodiments, the method comprises c) stirring the slurry of step b) to obtain stirred slurry. In some embodiments, the method comprises d) filtering the stirred slurry of step c) to obtain solid content. In some embodiments, the method comprises e) drying the solid content of step d).

In some aspect, provided are methods of preparing Compound 1 thiamine HCl co-crystal. In some embodiments, the method comprises a) mixing molar equivalents of Compound 1 and thiamine HCl. In some embodiments, the method comprises b) mixing ethanol and water with Compound 1 and thiamine HCl to form a slurry. In some embodiments, the method comprises c) stirring the slurry of step b) to obtain stirred slurry. In some embodiments, the method comprises d) filtering the stirred slurry of step c) to obtain solid content. In some embodiments, the method comprises e) drying the solid content of step d).

In some aspect, provided are methods of preparing Compound 1 ferric chloride co-crystal. In some embodiments, the method comprises a) mixing molar equivalents of Compound 1 and ferric chloride. In some embodiments, the method comprises b) mixing ethanol and water with Compound 1 and ferric chloride to form a slurry. In some embodiments, the method comprises c) stirring the slurry of step b) to obtain stirred slurry. In some embodiments, the method comprises d) filtering the stirred slurry of step c) to obtain solid content. In some embodiments, the method comprises drying the solid content of step d).

In some aspect, provided are methods of preparing Compound 1 manganese chloride co-crystal. In some embodiments, the method comprises a) mixing molar equivalents of Compound 1 and manganese chloride. In some embodiments, the method comprises b) mixing ethanol and water with Compound 1 and manganese chloride to form a slurry. In some embodiments, the method comprises c) stirring the slurry of step b) to obtain stirred slurry. In some embodiments, the method comprises d) filtering the stirred slurry of step c) to obtain solid content. In some embodiments, the method comprises e) drying the solid content of step d).

In some aspect, provided are methods of preparing Compound 1 zinc chloride co-crystal. In some embodiments, the method comprises a) mixing molar equivalents of Compound 1 and zinc chloride. In some embodiments, the method comprises b) mixing ethanol and water with Compound 1 and zinc chloride to form a slurry. In some embodiments, the method comprises c) stirring the slurry of step b) to obtain stirred slurry. In some embodiments, the method comprises d) filtering the stirred slurry of step c) to obtain solid content. In some embodiments, the method comprises e) drying the solid content of step d).

### BRIEF DESCRIPTION OF THE DRAWINGS

The present application can be understood by reference to the following description taken in conjunction with the accompanying figures.

FIG. 1A depicts X-ray powder diffraction data of Compound 1 calcium chloride co-crystal, according to at least some embodiments disclosed herein.

FIG. 1B depicts differential scanning calorimetry (DSC) analysis of Compound 1 calcium chloride co-crystal, according to at least some embodiments disclosed herein.

FIG. 1C depicts thermogravimetric analysis (TGA) of Compound 1 calcium chloride co-crystal, according to at least some embodiments disclosed herein.

FIG. 1D depicts dynamic vapor sorption (DVS) of Compound 1 calcium chloride co-crystal, according to at least some embodiments disclosed herein.

FIG. 1E depicts X-ray powder diffraction pattern of Compound 1 calcium chloride co-crystal after DVS analysis, according to at least some embodiments disclosed herein.

FIG. 1F depicts IR spectrum of Compound 1 calcium chloride co-crystal, according to at least some embodiments disclosed herein.

FIG. 1G depicts FT-Raman spectrum of Compound 1 calcium chloride co-crystal, according to at least some embodiments disclosed herein.

FIG. 1H depicts Low-Frequency (LF) Raman spectrum of Compound 1 calcium chloride co-crystal, according to at least some embodiments disclosed herein.

FIG. 1I depicts ¹H NMR spectrum of Compound 1 calcium chloride co-crystal, according to at least some embodiments disclosed herein.

FIG. 2A depicts X-ray powder diffraction data of Compound 1 pyridoxine HCl co-crystal, according to at least some embodiments disclosed herein.

FIG. 2B depicts differential scanning calorimetry (DSC) analysis of Compound 1 pyridoxine HCl co-crystal, according to at least some embodiments disclosed herein.

FIG. 2C depicts thermogravimetric analysis (TGA) of Compound 1 pyridoxine HCl co-crystal, according to at least some embodiments disclosed herein.

FIG. 2D depicts dynamic vapor sorption (DVS) of Compound 1 pyridoxine HCl co-crystal, according to at least some embodiments disclosed herein.

FIG. 2E depicts X-ray powder diffraction pattern of Compound 1 pyridoxine HCl co-crystal after DVS analysis, according to at least some embodiments disclosed herein.

FIG. 2F depicts IR spectrum of Compound 1 pyridoxine HCl co-crystal, according to at least some embodiments disclosed herein.

FIG. 2G depicts FT-Raman spectrum of Compound 1 pyridoxine HCl co-crystal, according to at least some embodiments disclosed herein.

FIG. 2H depicts Low-Frequency (LF) Raman spectrum of Compound 1 pyridoxine HCl co-crystal, according to at least some embodiments disclosed herein.

FIG. 2I depicts ¹H NMR spectrum of Compound 1 pyridoxine HCl co-crystal, according to at least some embodiments disclosed herein.

FIG. 3A depicts X-ray powder diffraction data of Compound 1 thiamine HCl co-crystal, according to at least some embodiments disclosed herein.

FIG. 3B depicts differential scanning calorimetry (DSC) analysis of Compound 1 thiamine HCl co-crystal, according to at least some embodiments disclosed herein.

FIG. 3C depicts thermogravimetric analysis (TGA) of Compound 1 thiamine HCl co-crystal, according to at least some embodiments disclosed herein.

FIG. 3D depicts dynamic vapor sorption (DVS) of Compound 1 thiamine HCl co-crystal, according to at least some embodiments disclosed herein.

FIG. 3E depicts X-ray powder diffraction pattern of Compound 1 thiamine HCl co-crystal after DVS analysis, according to at least some embodiments disclosed herein.

FIG. 3F depicts IR spectrum of Compound 1 thiamine HCl co-crystal, according to at least some embodiments disclosed herein.

FIG. 3G depicts FT-Raman spectrum of Compound 1 thiamine HCl co-crystal, according to at least some embodiments disclosed herein.

FIG. 3H depicts Low-Frequency (LF) Raman spectrum of Compound 1 thiamine HCl co-crystal, according to at least some embodiments disclosed herein.

FIG. 3I depicts ¹H NMR spectrum of Compound 1 thiamine HCl co-crystal, according to at least some embodiments disclosed herein.

FIG. 4A depicts X-ray powder diffraction data of Compound 1 ferric chloride co-crystal, according to at least some embodiments disclosed herein.

FIG. 4B depicts differential scanning calorimetry (DSC) analysis of Compound 1 ferric chloride co-crystal, according to at least some embodiments disclosed herein.

FIG. 4C depicts dynamic vapor sorption (DVS) of Compound 1 ferric chloride co-crystal, according to at least some embodiments disclosed herein.

FIG. 4D depicts ¹H NMR spectrum of Compound 1 ferric chloride co-crystal, according to at least some embodiments disclosed herein.

FIG. 5A depicts X-ray powder diffraction data of Compound 1 manganese chloride co-crystal, according to at least some embodiments disclosed herein.

FIG. 5B depicts Differential scanning calorimetry (DSC) analysis of Compound 1 manganese chloride co-crystal, according to at least some embodiments disclosed herein.

FIG. 5C depicts dynamic vapor sorption (DVS) of Compound 1 manganese chloride co-crystal, according to at least some embodiments disclosed herein.

FIG. 5D depicts ¹H NMR spectrum of Compound 1 manganese chloride co-crystal, according to at least some embodiments disclosed herein.

FIG. 6A depicts X-ray powder diffraction data of Compound 1 zinc chloride co-crystal, according to at least some embodiments disclosed herein.

FIG. 6B depicts differential scanning calorimetry (DSC) analysis of Compound 1 zinc chloride co-crystal, according to at least some embodiments disclosed herein.

FIG. 6C depicts dynamic vapor sorption (DVS) of Compound 1 zinc chloride co-crystal, according to at least some embodiments disclosed herein.

FIG. 6D depicts ¹H NMR spectrum of Compound 1 zinc chloride co-crystal, according to at least some embodiments disclosed herein.

### DETAILED DESCRIPTION

The following description is presented to enable a person of ordinary skill in the art to make and use the various embodiments. Descriptions of specific devices, techniques, and applications are provided only as examples. Various modifications to the examples described herein will be readily apparent to those of ordinary skill in the art, and the general principles defined herein may be applied to other examples and applications without departing from the spirit and scope of the various embodiments. Thus, the various embodiments are not intended to be limited to the examples described herein and shown, but are to be accorded the scope consistent with the claims.

Co-crystals may exhibit properties different from the free drug. The solid form may influence relevant physico-chemical parameters such as solubility, dissolution rate of the drug, chemical stability, melting point, and hygroscopicity, which can result in solids with superior properties.

As used herein and in the appended claims, the singular forms “a”, “an” and “the” include plural forms, unless the context clearly dictates otherwise.

As used herein, and unless otherwise specified, the terms “about” and “approximately,” when used in connection with doses, amounts, molar percent, or weight percent of ingredients of a composition or a dosage form, mean a dose, amount, molar percent, or weight percent that is recognized by those of ordinary skill in the art to provide a pharmacological effect equivalent to that obtained from the specified dose, amount, molar percent, or weight percent. Specifically, the terms “about” and “approximately,” when used in this context, contemplate a dose, amount, molar percent, or weight percent within 15%, within 10%, within 5%, within 4%, within 3%, within 2%, within 1%, or within 0.5% of the specified dose, amount, molar percent, or weight percent.

As used herein, “therapeutically effective amount” or “effective amount” indicates an amount that results in a desired pharmacological and/or physiological effect for the condition. The effect may be prophylactic in terms of completely or partially preventing a condition or symptom thereof and/or may be therapeutic in terms of a partial or complete cure for the condition and/or adverse effect attributable to the condition.

As used herein, the term “pharmaceutically acceptable excipient,” and cognates thereof, refers to adjuvants, binders, diluents, etc. known to the skilled artisan that are suitable for administration to an individual (e.g., a mammal or non-mammal). Combinations of two or more excipients are also contemplated. The pharmaceutically acceptable excipient(s) and any additional components, as described herein, should be compatible for use in the intended route of administration (e.g., oral, parenteral) for a particular dosage form, as would be recognized by the skilled artisan.

The terms “treat,” “treating,” and “treatment” are meant to include alleviating or abrogating a disorder, disease, or condition, or one or more of the symptoms associated with the disorder, disease, or condition; or to slowing the progression, spread or worsening of a disease, disorder or condition or of one or more symptoms thereof. Often, the beneficial effects that a subject derives from a therapeutic agent do not result in a complete cure of the disease, disorder or condition.

The term “subject” refers to an animal, including, but not limited to, a primate (e.g., human), monkey, cow, pig, sheep, goat, horse, dog, cat, rabbit, rat, or mouse. The terms “subject” and “patient” are used interchangeably herein in reference, for example, to a mammalian subject, such as a human.

As used herein, the term “substantially as shown in” when referring, for example, to an X-Ray Powder Diffraction (XRPD) pattern or a Differential Scanning Calorimetry (DSC), includes a pattern or graph that is not necessarily identical to those depicted herein, but that falls within the limits of experimental error or deviations when considered by one of ordinary skill in the art.

### I. Co-Crystals

Co-crystals may exhibit properties different from the free drug. The solid form may influence relevant physico-chemical parameters, such as solubility, dissolution rate of the drug, chemical stability, melting point, and hygroscopicity, which can result in solids with superior properties.

In one aspect, provided herein are co-crystals of 2-bromo-1-(3,3-dinitroazetidin-1-yl)ethanone (“Compound 1”):

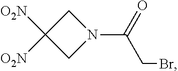 [SMILES: O=C(CBr)N1CC([N+](=O)[O-])([N+](=O)[O-])C1]

and calcium chloride, pyridoxine HCl, thiamine HCl, ferric chloride, manganese chloride, or zinc chloride. In some embodiments, co-crystals described herein can have a purity of at least 80%, 85%, 90%, 91%, 92%, 93%, 94%, 95%, 96%, 97%, 98%, 99%, 99.5%, 99.8%, or 99.9% by weight. In some embodiments, co-crystals provided herein can be characterized by XRPD, DSC, TGA, DVS, IR spectroscopy, FT-Raman spectroscopy, LF Raman spectroscopy, or NMR spectroscopy, or any combination thereof.

In some embodiments, formulated drug products comprising any of the co-crystals described herein can be analyzed by XRPD to identify the co-crystal in the product. In some embodiments, this analysis and identification can be achieved by deconvoluting the XRPD data obtained from the formulated product. In some embodiments, this deconvolution can be achieved by subtracting the known excipient signals from the XRPD data of the formulated product. In some embodiments, various chemometrics techniques can be used to identify the co-crystal in the product, for example, principal component analysis. Such analysis may be required to identify XRPD peaks belonging to the co-crystal as they may overlap with XRPD peaks of some of the excipients used in the formulation.

For formulated drug products comprising any of the co-crystals described herein, the formulated products can be analyzed by IR and or Raman spectroscopy to identify the co-crystal in the product. This can be achieved by deconvoluting the IR and/or Raman spectroscopy obtained from the formulated product. This can be achieved by subtracting the known excipient signals from the IR and/or Raman spectroscopy of the formulated product. In addition, various chemometrics techniques can be used to identify the co-crystal in the product, for example principal component analysis. Such analysis may be required to identify IR and/or Raman signals belonging to the co-crystal as they may overlap with IR and/or Raman signals of some of the excipients used in the formulation.

In some embodiments, the solubility in water or in a mixture of water with other solvents of any of the co-crystals described herein may be higher than the solubility of an amorphous or crystalline form of Compound 1. In some embodiments, the solubility of the co-crystals described herein may be significantly higher than the solubility of an amorphous or crystalline form of Compound 1. In some embodiments, the solubility of any of the co-crystals described herein is at least about 1%, 5%, 10%, 15%, 20%, 30%, 40%, 50%, 60%, 70%, 80%, 90%, 100%, 150%, 200%, 250%, 300%, 400%, 500%, 600%, 700%, 800%, or 900% higher than the solubility of an amorphous or crystalline form of Compound 1. In some embodiments, solubility is measured as gram soluble in 100 g water.

In some embodiments, the co-crystal is impact-insensitive or not impact-sensitive. A material can be tested for impact sensitivity using one or more standard protocols, for example, as described in the United Nations Manual of Tests and Criteria, seventh edition, 2019 (Orange Book). For example, the explosiveness of a compound, for example, a crystalline form of Compound 1, can be determined by a Series 3 Type (a)(ii) Test procedure using a BAM Fallhammer, as described in the Orange Book. In some embodiments, tests are conducted such that 40 mm³ of sample disposed in an impact device is subjected to about 40 J of energy (e.g., using a drop weight of about 10 kg dropped from a height of about 40 cm). The test is conducted on six separate samples under the same experimental conditions, and the operator determines whether an explosion has occurred. If no explosion occurs on all six samples, then the material is characterized as not impact-sensitive or impact-insensitive. However, if one of the samples fails the test and causes an explosion, then the material is characterized as impact-sensitive.

### 1. Compound 1 Calcium Chloride Co-Crystal

In one aspect, provided herein is a co-crystal of Compound 1 and calcium chloride.

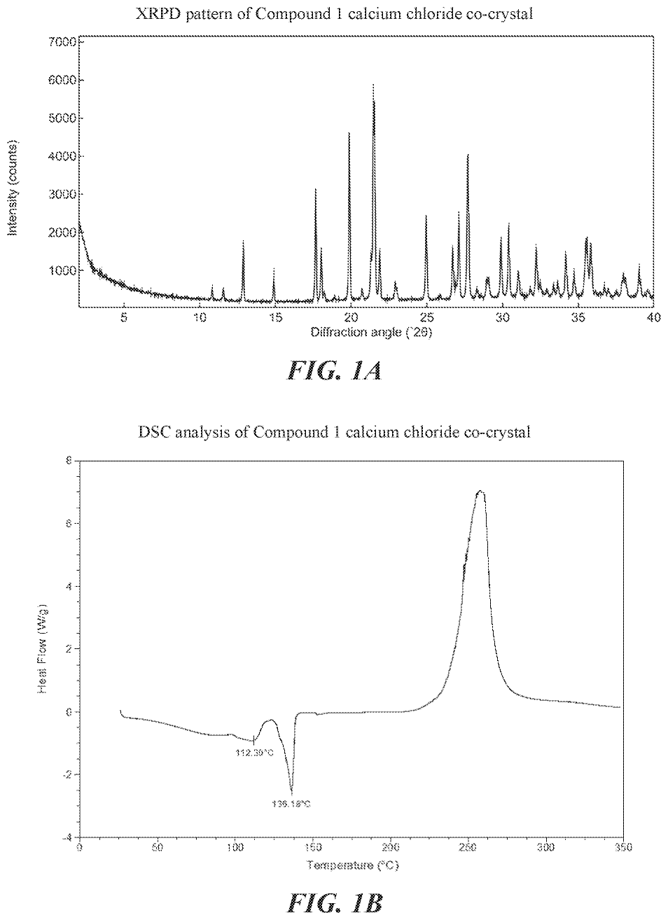

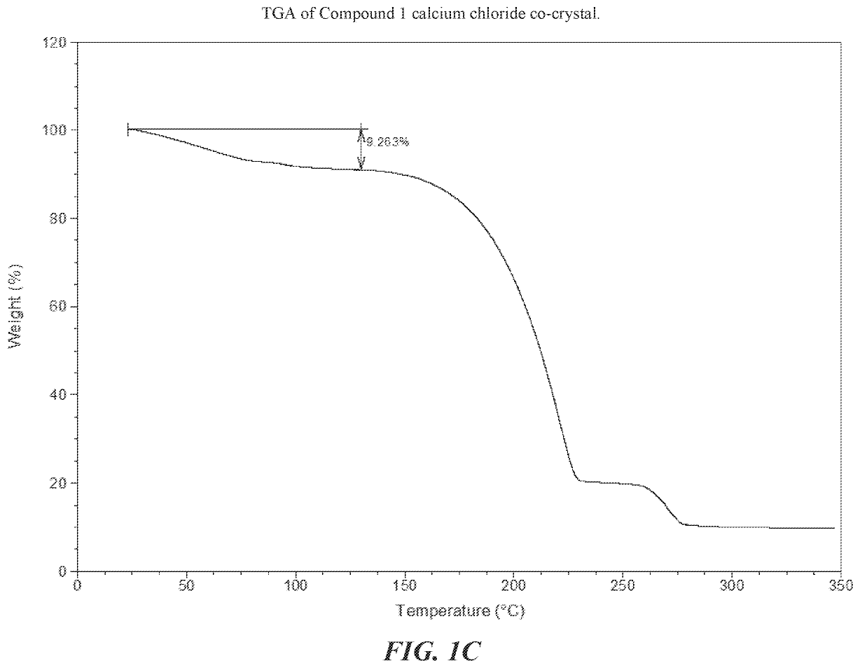

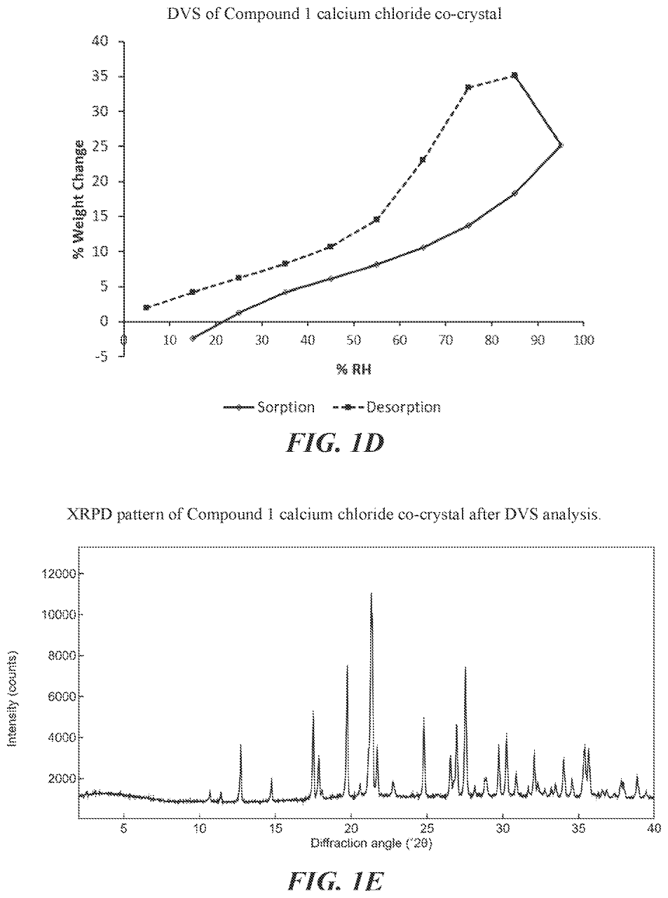

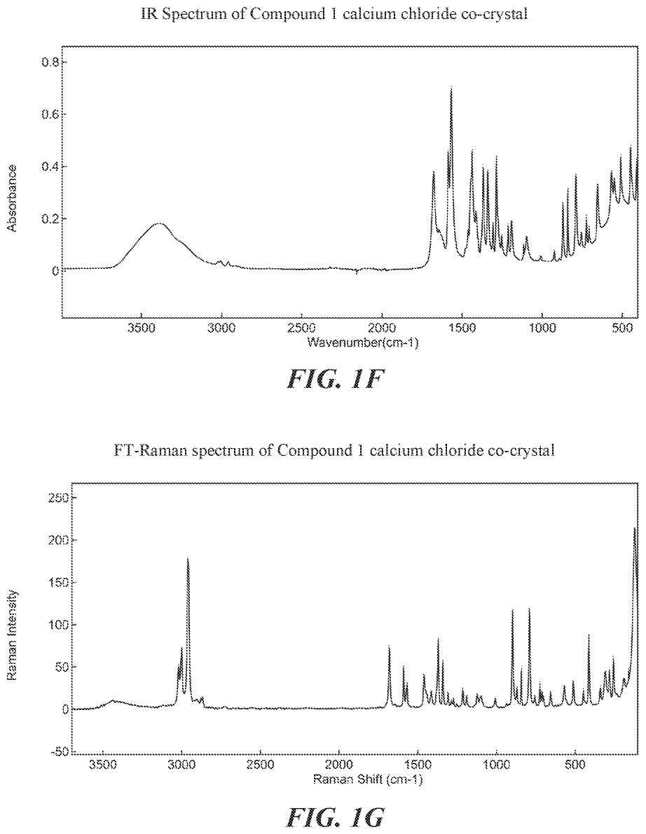

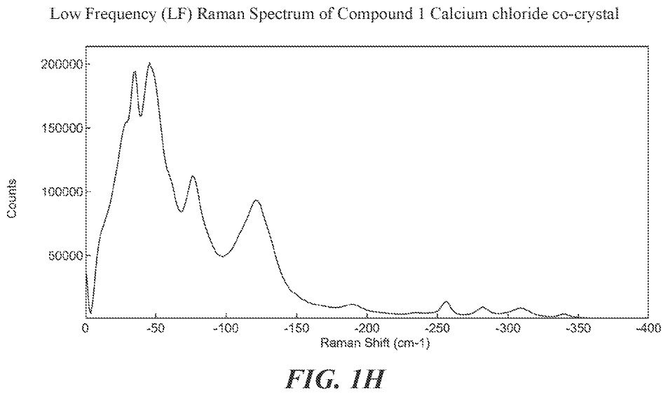

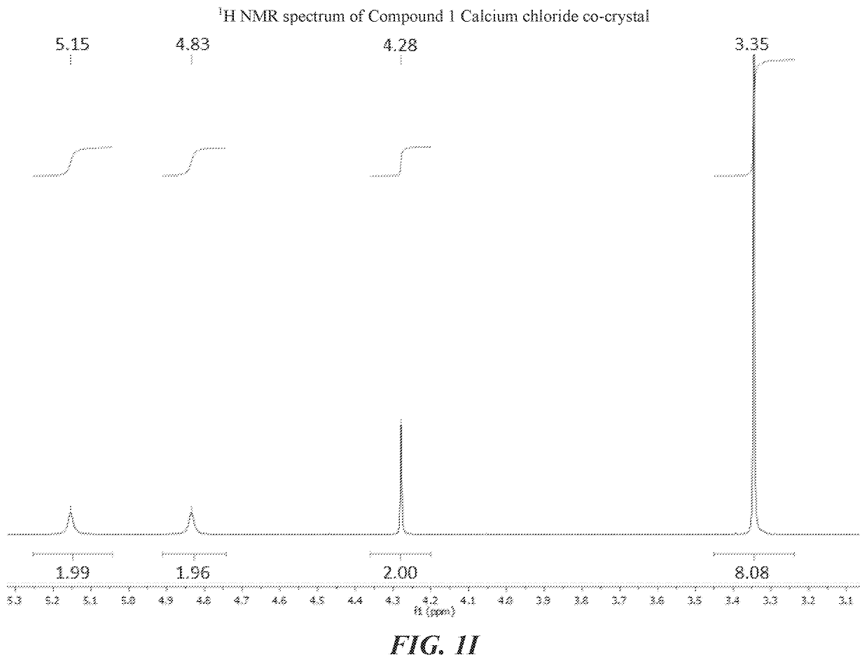

In some embodiments, the Compound 1 calcium chloride co-crystal has an XRPD pattern displaying at least one, at least two, at least three, at least four, at least five, at least six, at least seven, at least eight, at least nine, at least ten or more of the peaks at angles 2-theta in the XRPD pattern substantially as shown in FIG. 1A or as provided in Table 1B. In some embodiments, the Compound 1 calcium chloride co-crystal has an XRPD pattern displaying at least one, at least two, at least three, at least four, at least five, at least six, at least seven, at least eight, at least nine, at least ten or more of the peaks at angles 2-theta with the greatest intensity in the XRPD pattern substantially as shown in FIG. 1A or as provided in Table 1B. It should be understood that relative intensities can vary depending on a number of factors, including sample preparation, mounting, and the instrument and analytical procedure and settings used to obtain the spectrum. Relative peak intensities and peak assignments can vary within experimental error. In some embodiments, peak assignments listed herein, including for the Compound 1 calcium chloride co-crystal, can vary by about ±2.0 degrees, ±1.5 degrees, ±1.0 degrees, ±0.9 degrees, ±0.8 degrees, ±0.7 degrees, ±0.6 degrees, ±0.5 degrees, ±0.4 degrees, ±0.3 degrees, ±0.2 degrees, or ±0.1 degrees 2-theta.

In some embodiments, the Compound 1 calcium chloride co-crystal has an XRPD pattern comprising one or more peaks at angles 2-theta of about 10.8±δx, 11.6±δx, and 14.9±δx degrees. In some embodiments, the Compound 1 calcium chloride co-crystal has an XRPD pattern comprising peaks at angles 2-theta of about 10.8±δx, 11.6±δx, 12.9±δx, and 14.9±δx degrees. In some embodiments, the Compound 1 calcium chloride co-crystal has an XRPD pattern comprising peaks at angles 2-theta of about 10.8±δx, 11.6±δx, 12.9±δx, 14.9±δx, 17.7±δx, 18.0±δx, 18.2±δx, 18.9±δx, 19.2±δx, 19.9±δx, 20.7±δx, 21.5±δx, 21.9±δx, 22.9±δx, 25.0±δx, 25.9±δx, 26.7±δx, 27.1±δx, 27.7±δx, 28.3±δx, 29.0±δx, 29.1±δx, 29.9±δx, 30.4±δx, 31.0±δx, 31.8±δx, 32.2±δx, 32.5±δx, 32.9±δx, 33.4±δx, 33.6±δx, 34.2±δx, 34.7±δx, 35.5±δx, 35.6±δx, 35.8±δx, 36.7±δx, 37.0±δx, 37.5±δx, 38.0±δx, or 39.0±δx degrees, or any combination thereof. In some embodiments, δx is 0 degrees 2-theta. In some embodiments, δx is 0.2 degrees 2-theta. In some embodiments, δx is 0.1, 0.2, 0.3, 0.4, 0.5, 0.6, 0.7, 0.8, 0.9, 1.0, 1.5, 2.0, 2.5, 3.0, 4.0, 5.0, 6.0, 7.0, 8.0, 9.0, or 10 degrees 2-theta.

In some embodiments, the Compound 1 calcium chloride co-crystal has a differential scanning calorimetry (DSC) graph substantially as shown in FIG. 1B. In some embodiments, the Compound 1 calcium chloride co-crystal is characterized by having a DSC graph comprising one or more endothermic peaks at about 112.3 or 136.2° C. In some embodiments, the Compound 1 calcium chloride co-crystal is characterized by having a DSC graph comprising one or more endothermic peaks at about 112.3° C.±δ_{DSC} or 136.2° C.±δ_{DSC}, where δ_{DSC} is 0.1, 0.2, 0.3, 0.4, 0.5, 0.6, 0.7, 0.8, 0.9, 1, 1.25, 1.5, 1.75, 2, 2.5, 3, 3.5, 4, 4.5, 5, 6, 7, 8, 9, or 10° C. In some embodiments, δ_{DSC} is 2° C. In some embodiments, the Compound 1 calcium chloride co-crystal is characterized by having a DSC graph comprising one or more endothermic peaks between about 110° C. and about 114° C. or between about 134° C. and about 138° C.

In some embodiments, Compound 1 calcium chloride co-crystal, when heated, loses volatiles. In some embodiments, Compound 1 calcium chloride co-crystal, when heated, loses water. In some embodiments, the Compound 1 calcium chloride co-crystal melts at or has a melting point at about 112.3 or 136.2° C. as determined by DSC. In some embodiments, the Compound 1 calcium chloride co-crystal melts at or has a melting point at about 112.3° C.±δ_{DSC} or 136.2° C.±δ_{DSC} as determined by DSC, where δ_{DSC} is 0.1, 0.2, 0.3, 0.4, 0.5, 0.6, 0.7, 0.8, 0.9, 1, 1.25, 1.5, 1.75, 2, 2.5, 3, 3.5, 4, 4.5, 5, 6, 7, 8, 9, or 10° C. In some embodiments, δ_{DSC} is 2° C. In some embodiments, the Compound 1 calcium chloride co-crystal melts between 110° C. and 114° C. or between 134° C. and 138° C. as determined by DSC.

In some embodiments, Compound 1 calcium chloride co-crystal has a thermogravimetric analysis (TGA) graph substantially as shown in FIG. 1C. In some embodiments, Compound 1 calcium chloride co-crystal is hydrated. In some embodiments, % weight decrease of Compound 1 calcium chloride co-crystal as determined by TGA from about 25° C. to about 130° C. is about 9.3%. In some embodiments, % weight decrease of Compound 1 calcium chloride co-crystal as determined by TGA from about 25° C. to about 130° C. is at least about 9%. In some embodiments, % weight decrease of Compound 1 calcium chloride co-crystal as determined by TGA from about 25° C. to about 130° C. is about 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 10.5%, 11%, 11.5%, 12%, 12.5%, 13%, 13.5%, 14%, 14.5%, 15%, 16%, 17%, 18%, 19%, or 20%. In some embodiments, % weight decrease of Compound 1 calcium chloride co-crystal as determined by TGA from about 25° C. to about 130° C. is at least about 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 10.5%, 11%, 11.5%, 12%, 12.5%, 13%, 13.5%, 14%, 14.5%, 15%, 16%, 17%, 18%, 19%, or 20%.

In some embodiments, Compound 1 calcium chloride co-crystal has a dynamic vapor sorption (DVS) graph substantially as shown in FIG. 1D. In some embodiments, Compound 1 calcium chloride co-crystal is hygroscopic. In some embodiments, Compound 1 calcium chloride co-crystal is reversibly hygroscopic. In some embodiments, Compound 1 calcium chloride co-crystal does not undergo hysteresis. In some embodiments, Compound 1 calcium chloride co-crystal is stable under various humidity conditions. In some embodiments, % weight increase of Compound 1 calcium chloride co-crystal recovers to about 5% or less after a 15% RH→95% RH→15% RH DVS cycle. In some embodiments, crystalline form of Compound 1 calcium chloride co-crystal after a DVS analysis exposing Compound 1 calcium chloride co-crystal from 5% RH to 95% RH.

In some embodiments, % weight increase of Compound 1 calcium chloride co-crystal as determined by DVS at about 95% RH is about 25%. In some embodiments, % weight increase of Compound 1 calcium chloride co-crystal as determined by DVS from about 35% RH to about 65% RH is at least 5%. In some embodiments, % weight increase of Compound 1 calcium chloride co-crystal as determined by DVS from about 35% RH to about 65% RH is at least 1, 2, 3, 4, 5, 6, 7, 8, 9, 10, 11, 12, 13, 14, 15, 16, 17, 18, 19, 20, 25, 30, 35, 40, 45, or 50%. In some embodiments, % weight increase of Compound 1 calcium chloride co-crystal as determined by DVS from about 35% RH to about 65% RH is about 1, 2, 3, 4, 5, 6, 7, 8, 9, 10, 11, 12, 13, 14, 15, 16, 17, 18, 19, 20, 25, 30, 35, 40, 45, or 50%.

In some embodiments, Compound 1 calcium chloride co-crystal has an IR spectrum displaying at least one, at least two, at least three, at least four, at least five, at least six, at least seven, at least eight, at least nine, at least ten or more of the peaks in the IR spectrum substantially as shown in FIG. 1F or as provided in Table 1E.

In some embodiments, the Compound 1 calcium chloride co-crystal has an IR spectrum displaying at least one, at least two, at least three, at least four, at least five, at least six, at least seven, at least eight, at least nine, at least ten or more of the peaks at angles 2-theta with the greatest intensity in the IR spectrum substantially as shown in FIG. 1F or as provided in Table 1E. It should be understood that relative intensities can vary depending on a number of factors, including sample preparation, mounting, and the instrument and analytical procedure and settings used to obtain the spectrum. Relative peak intensities and peak assignments can vary within experimental error. In some embodiments, peak assignments listed herein, including for the Compound 1 calcium chloride co-crystal, can vary by about ±0.1, ±0.2, ±0.3, ±0.4, ±0.5, ±0.6, ±0.7, ±0.8, ±0.9, ±0.10, ±0.15, ±0.2, ±0.25, ±0.3, ±0.35, ±0.4, ±0.45, ±0.5, ±0.75, ±1, ±1.25, ±1.5, ±2, ±2.5, ±3, ±3.5, ±4, ±4.5, ±5, +6, ±7, ±8, ±9, or ±10 cm⁻¹. In some embodiments, peak assignments listed herein, including for the Compound 1 calcium chloride co-crystal, can vary by about ±2 cm⁻¹.

In some embodiments, the Compound 1 calcium chloride co-crystal has an IR spectrum comprising one or more peaks at about 411, 450, 510, 551, 568, 654, 708, 724, 757, 790, 841, 871, 897, 926, 1009, 1097, 1116, 1191, 1214, 1253, 1285, 1308, 1339, 1369, 1414, 1438, 1462, 1567, 1586, 1644, 1679, 2956, 3005, 3020, or 3387 cm⁻¹, or any combination thereof. In some embodiments, the Compound 1 calcium chloride co-crystal has an IR spectrum comprising peaks at about 450, 510, 654, 790, 841, 871, 1285, 1339, 1369, 1438, 1567, and 1679 cm⁻¹. In some embodiments, the Compound 1 calcium chloride co-crystal has an IR spectrum comprising one or more peaks at about 411±δ_{IR}, 450±δ_{IR}, 510±δ_{IR}, 551±δ_{IR}, 568±δ_{IR}, 654±δ_{IR}, 708±δ_{IR}, 724±δ_{IR}, 757±δ_{IR}, 790±δ_{IR}, 841±δ_{IR}, 871±δ_{IR}, 897±δ_{IR}, 926±δ_{IR}, 1009±δ_{IR}, 1097±δ_{IR}, 1116±δ_{IR}, 1191±δ_{IR}, 1214±δ_{IR}, 1253±δ_{IR}, 1285±δ_{IR}, 1308±δ_{IR}, 1339±δ_{IR}, 1369±δ_{IR}, 1414±δ_{IR}, 1438±δ_{IR}, 1462±δ_{IR}, 1567±δ_{IR}, 1586±δ_{IR}, 1644±δ_{IR}, 1679±δ_{IR}, 2956±δ_{IR}, 3005±δ_{IR}, 3020±δ_{IR}, or 3387±δ_{IR} cm⁻¹, or any combination thereof. In some embodiments, the Compound 1 calcium chloride co-crystal has an IR spectrum comprising peaks at about 450±δ_{IR}, 510±δ_{IR}, 654±δ_{IR}, 790±δ_{IR}, 841±δ_{IR}, 871±δ_{IR}, 1285±δ_{IR}, 1339±δ_{IR}, 1369±δ_{IR}, 1438±δ_{IR}, 1567±δ_{IR}, and 1679±δ_{IR} cm⁻¹. In some embodiments, δ_{IR} is 0.1, 0.2, 0.3, 0.4, 0.5, 0.6, 0.7, 0.8, 0.9, 0.10, 0.15, 0.2, 0.25, 0.3, 0.35, 0.4, 0.45, 0.5, 0.75, 1, 1.25, 1.5, 2, 2.5, 3, 3.5, 4, 4.5, 5, 6, 7, 8, 9, or 10 cm⁻¹. In some embodiments. δ_{IR} is 2 cm⁻¹.

In some embodiments, Compound 1 calcium chloride co-crystal has an FT-Raman spectrum displaying at least one, at least two, at least three, at least four, at least five, at least six, at least seven, at least eight, at least nine, at least ten or more of the peaks in the FT-Raman spectrum substantially as shown in FIG. 1G or as provided in Table 1F.

In some embodiments, the Compound 1 calcium chloride co-crystal has an FT-Raman spectrum displaying at least one, at least two, at least three, at least four, at least five, at least six, at least seven, at least eight, at least nine, at least ten or more of the peaks at angles 2-theta with the greatest intensity in the FT-Raman spectrum substantially as shown in FIG. 1G or as provided in Table 1F. It should be understood that relative intensities can vary depending on a number of factors, including sample preparation, mounting, and the instrument and analytical procedure and settings used to obtain the spectrum. Relative peak intensities and peak assignments can vary within experimental error. In some embodiments, peak assignments listed herein, including for the Compound 1 calcium chloride co-crystal, can vary by about ±0.1, ±0.2, ±0.3, ±0.4, ±0.5, ±0.6, ±0.7, ±0.8, ±0.9, ±0.10, ±0.15, ±0.2, ±0.25, ±0.3, ±0.35, ±0.4, ±0.45, ±0.5, ±0.75, ±1, ±1.25, ±1.5, ±2, ±2.5, ±3, ±3.5, ±4, ±4.5, ±5, ±6, ±7, ±δ, ±9, or ±10 cm⁻¹. In some embodiments, peak assignments listed herein, including for the Compound 1 calcium chloride co-crystal, can vary by about ±2 cm⁻¹.

In some embodiments, the Compound 1 calcium chloride co-crystal has an FT-Raman spectrum comprising one or more peaks at about 122, 161, 190, 257, 282, 309, 340, 413, 448, 512, 570, 656, 710, 724, 757, 791, 841, 870, 897, 1009, 1098, 1123, 1189, 1214, 1252, 1273, 1287, 1308, 1341, 1370, 1414, 1461, 1569, 1590, 1680, 2866, 2961, 2999, or 3019 cm⁻¹, or any combination thereof. In some embodiments, the Compound 1 calcium chloride co-crystal has an FT-Raman spectrum comprising peaks at about 413, 791, 897, 1370, 1680, and 2961 cm⁻¹. In some embodiments, the Compound 1 calcium chloride co-crystal has an FT-Raman spectrum comprising one or more peaks at about 122±δ_{FT}, 161±δ_{FT}, 190±δ_{FT}, 257±δ_{FT}, 282±δ_{FT}, 309±δ_{FT}, 340±δ_{FT}, 413±δ_{FT}, 448±δ_{FT}, 512±δ_{FT}, 570±δ_{FT}, 656±δ_{FT}, 710±δ_{FT}, 724±δ_{FT}, 757±δ_{FT}, 791±δ_{FT}, 841±δ_{FT}, 870±δ_{FT}, 897±δ_{FT}, 1009±δ_{FT}, 1098±δ_{FT}, 1123±δ_{FT}, 1189±δ_{FT}, 1214±δ_{FT}, 1252±δ_{FT}, 1273±δ_{FT}, 1287±δ_{FT}, 1308±δ_{FT}, 1341±δ_{FT}, 1370±δ_{FT}, 1414±δ_{FT}, 1461±δ_{FT}, 1569±δ_{FT}, 1590±δ_{FT}, 1680±δ_{FT}, 2866±δ_{FT}, 2961±δ_{FT}, 2999±δ_{FT}, or 3019±δ_{FT} cm⁻¹, or any combination thereof. In some embodiments, the Compound 1 calcium chloride co-crystal has an FT-Raman spectrum comprising one or more peaks at about 413±δ_{FT}, 791δ_{FT}, 897δ_{FT}, 1370±δ_{FT}, 1680δ_{FT}, and 2961±δ_{FT} cm⁻¹. In some embodiments, δ_{FT} is 0.1, 0.2, 0.3, 0.4, 0.5, 0.6, 0.7, 0.8, 0.9, 0.10, 0.15, 0.2, 0.25, 0.3, 0.35, 0.4, 0.45, 0.5, 0.75, 1, 1.25, 1.5, 2, 2.5, 3, 3.5, 4, 4.5, 5, 6, 7, 8, 9, or 10 cm⁻¹. In some embodiments, δ_{FT} is 2 cm⁻¹.

In some embodiments, Compound 1 calcium chloride co-crystal has an LF-Raman spectrum displaying at least one, at least two, at least three, at least four, at least five, at least six, at least seven, at least eight, at least nine, at least ten or more of the peaks in the LF-Raman spectrum substantially as shown in FIG. 1H or as provided in Table 1H.

In some embodiments, the Compound 1 calcium chloride co-crystal has an LF-Raman spectrum displaying at least one, at least two, at least three, at least four, at least five, at least six, at least seven, at least eight, at least nine, at least ten or more of the peaks at angles 2-theta with the greatest intensity in the LF-Raman spectrum substantially as shown in FIG. 1H or as provided in Table 1H. It should be understood that relative intensities can vary depending on a number of factors, including sample preparation, mounting, and the instrument and analytical procedure and settings used to obtain the spectrum. Relative peak intensities and peak assignments can vary within experimental error. In some embodiments, peak assignments listed herein, including for the Compound 1 calcium chloride co-crystal, can vary by about ±0.1, ±0.2, ±0.3, ±0.4, ±0.5, ±0.6, ±0.7, ±0.8, ±0.9, ±0.10, ±0.15, ±0.2, ±0.25, ±0.3, ±0.35, ±0.4, ±0.45, ±0.5, ±0.75, ±1, ±1.25, ±1.5, ±2, ±2.5, ±3, ±3.5, ±4, ±4.5, ±5, ±6, ±7, ±8, ±9, or ±10 cm⁻¹. In some embodiments, peak assignments listed herein, including for the Compound 1 calcium chloride co-crystal, can vary by about ±2 cm⁻¹.

In some embodiments, the Compound 1 calcium chloride co-crystal has an LF-Raman spectrum comprising one or more peaks at about −340, −309, −282, −256, −189, −121, −76, −46, or −35 cm⁻¹, or any combination thereof. In some embodiments, the Compound 1 calcium chloride co-crystal has an LF-Raman spectrum comprising peaks at about −256, −189, −121, −76, −46, and −35 cm⁻¹. In some embodiments, the Compound 1 calcium chloride co-crystal has an LF-Raman spectrum comprising one or more peaks at about −510±δ_{LF}, −448±δ_{LF}, −412±δ_{LF}, −340±δ_{LF}, −309±δ_{LF}, −282±δ_{LF}, −256±δ_{LF}, −189±δ_{LF}, −121±δ_{LF}, −76±δ_{LF}, −46±δ_{LF}, or −35±δ_{LF} cm⁻¹, or any combination thereof. In some embodiments, the Compound 1 calcium chloride co-crystal has an LF-Raman spectrum comprising peaks at about −256±δ_{LF}, −189±δ_{LF}, −121±δ_{LF}, −76±δ_{LF}, −46±δ_{LF}, and −35±δ_{LF} cm⁻¹. In some embodiments, δ_{LF} is 0.1, 0.2, 0.3, 0.4, 0.5, 0.6, 0.7, 0.8, 0.9, 0.10, 0.15, 0.2, 0.25, 0.3, 0.35, 0.4, 0.45, 0.5, 0.75, 1, 1.25, 1.5, 2, 2.5, 3, 3.5, 4, 4.5, 5, 6, 7, 8, 9, or 10 cm⁻¹. In some embodiments, δ_{LF} is 2 cm⁻¹.

In some embodiments, Compound 1 calcium chloride co-crystal has a ¹H NMR spectrum substantially as shown in FIG. 1I. In some embodiments, the Compound 1 calcium chloride co-crystal has ¹H NMR spectrum comprising one or more peaks at about 3.35, 4.28, 4.83, or 5.15 ppm, or any combination thereof. In some embodiments, the Compound 1 calcium chloride co-crystal has an ¹H NMR spectrum comprising one or more peaks at about 3.35±δ_{N}, 4.28±δ_{N}, 4.83±δ_{N}, or 5.15±δ_{N} ppm, or any combination thereof. In some embodiments, δ_{N} is 0.01, 0.05, 0.1, 0.2, 0.3, 0.4, 0.5, 0.6, 0.7, 0.8, 0.9, 0.10, 0.15, 0.2, 0.25, 0.3, 0.35, 0.4, 0.45, 0.5, 0.75, 1, 1.25, 1.5, 2, 3, 4, or 5 ppm. In some embodiments, δ_{N} is 0.2 ppm.

In some embodiments, Compound 1 calcium chloride co-crystal is hygroscopic. In some embodiments, Compound 1 calcium chloride co-crystal is unsolvated. In some embodiments, Compound 1 calcium chloride co-crystal is hydrated. In some embodiments, Compound 1 calcium chloride co-crystal is not impact-sensitive. In some embodiments, Compound 1 calcium chloride co-crystal is less impact-sensitive than a Compound 1 crystal.

In some embodiments, the solubility of Compound 1 calcium chloride co-crystal in water or in a mixture of water with other solvents is higher than the solubility of an amorphous or crystalline form of Compound 1. In some embodiments, the solubility of Compound 1 calcium chloride co-crystal herein may be significantly higher than the solubility of an amorphous or crystalline form of Compound 1. In some embodiments, the solubility of Compound 1 calcium chloride co-crystal is at least about 1%, 5%, 10%, 15%, 20%, 30%, 40%, 50%, 60%, 70%, 80%, 90%, 100%, 150%, 200%, 250%, 300%, 400%, 500%, 600%, 700%, 800%, or 900% higher than the solubility of an amorphous or crystalline form of Compound 1. In some embodiments, solubility is measured as grams of Compound 1 calcium chloride co-crystal soluble in 100 g water.

### 2. Compound 1 Pyridoxine HCl Co-Crystal

In one aspect, provided herein is a co-crystal of Compound 1 and pyridoxine HCl.

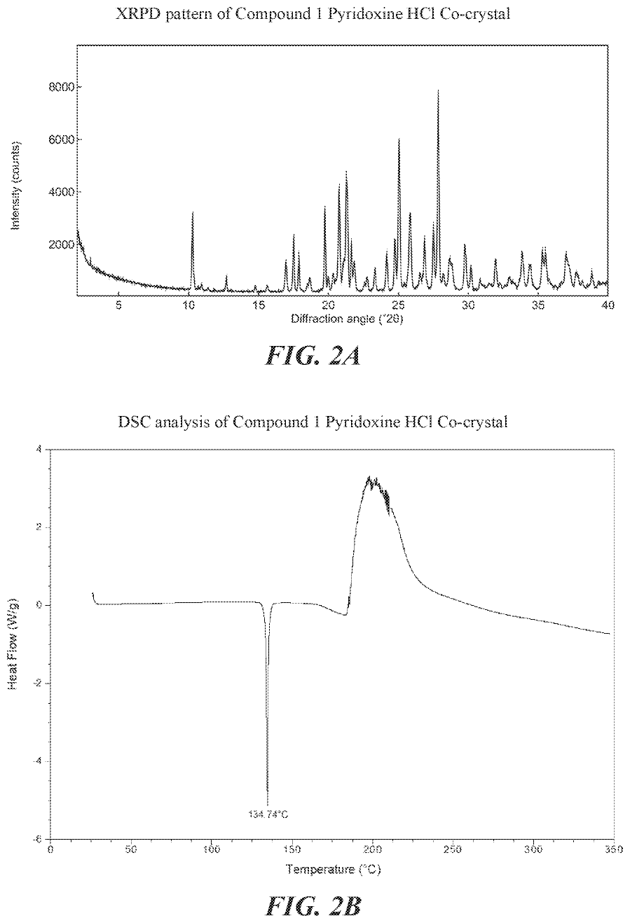

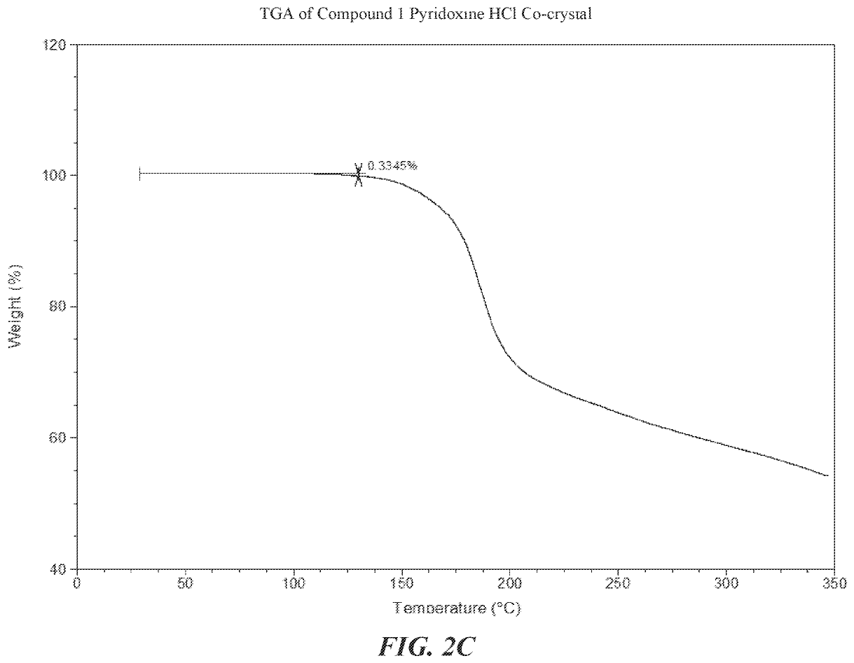

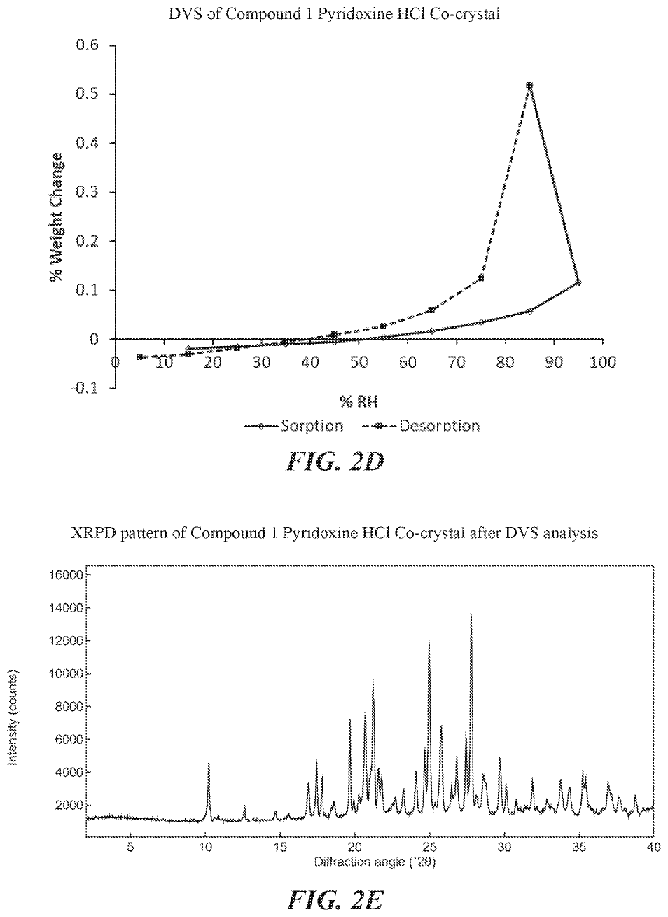

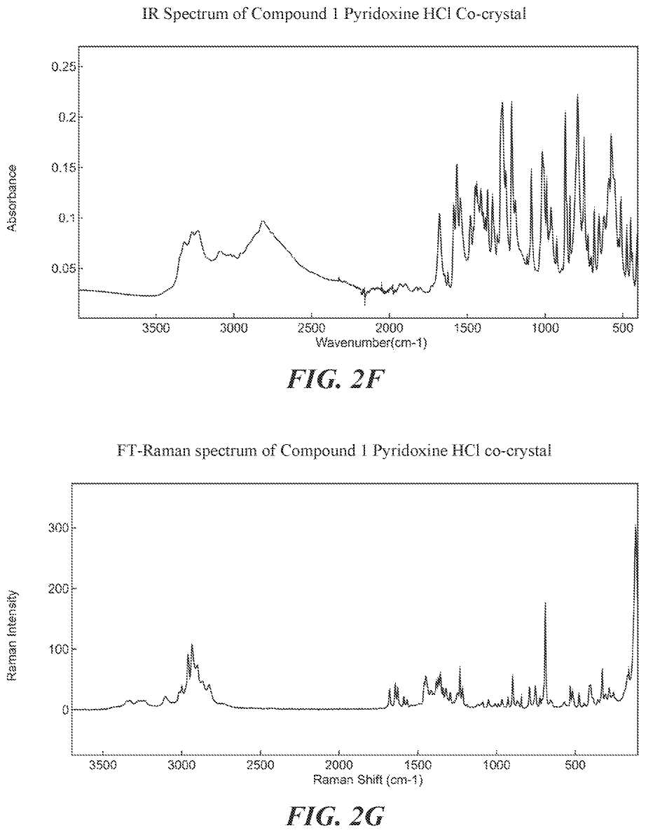

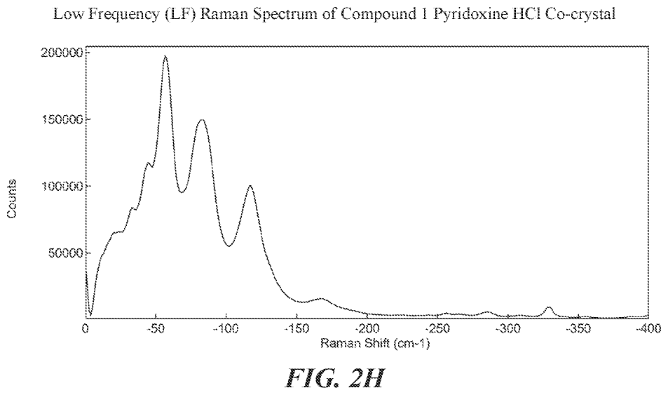

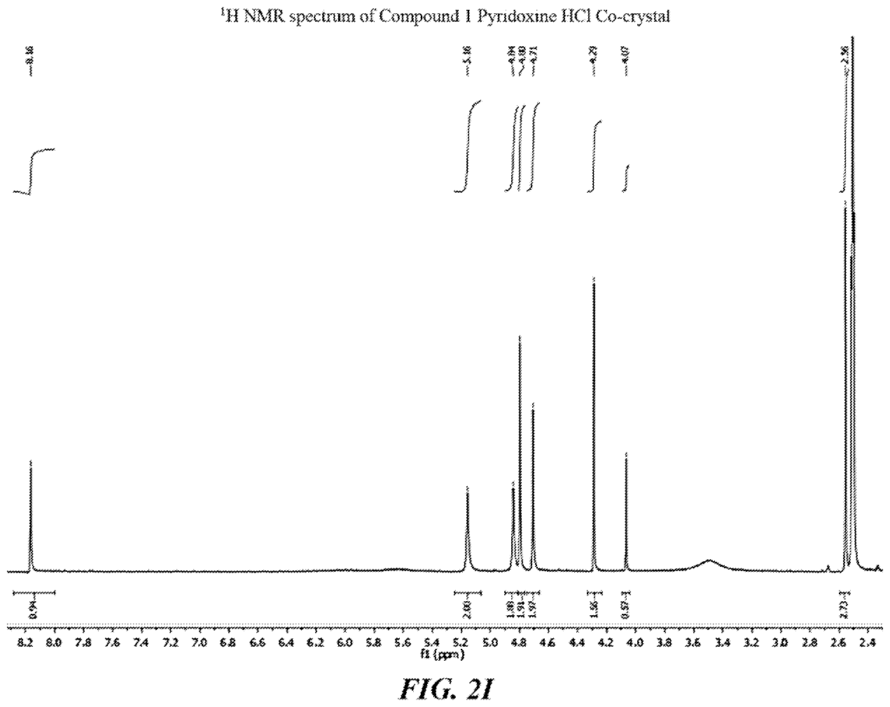

In some embodiments, the Compound 1 pyridoxine HCl co-crystal has an XRPD pattern displaying at least one, at least two, at least three, at least four, at least five, at least six, at least seven, at least eight, at least nine, at least ten or more of the peaks at angles 2-theta in the XRPD pattern substantially as shown in FIG. 2A or as provided in Table 2B. In some embodiments, the Compound 1 pyridoxine HCl co-crystal has an XRPD pattern displaying at least one, at least two, at least three, at least four, at least five, at least six, at least seven, at least eight, at least nine, at least ten or more of the peaks at angles 2-theta with the greatest intensity in the XRPD pattern substantially as shown in FIG. 2A or as provided in Table 2B. It should be understood that relative intensities can vary depending on a number of factors, including sample preparation, mounting, and the instrument and analytical procedure and settings used to obtain the spectrum. Relative peak intensities and peak assignments can vary within experimental error. In some embodiments, peak assignments listed herein, including for the Compound 1 pyridoxine HCl co-crystal, can vary by about ±2.0 degrees, ±1.5 degrees, ±1.0 degrees, ±0.9 degrees, ±0.8 degrees, ±0.7 degrees, ±0.6 degrees, ±0.5 degrees, ±0.4 degrees, ±0.3 degrees, ±0.2 degrees, or ±0.1 degrees 2-theta.

In some embodiments, the Compound 1 pyridoxine HCl co-crystal has an XRPD pattern comprising one or more peaks at angles 2-theta of about 11.4±δx, 12.7±δx, and 14.8±δx degrees. In some embodiments, the Compound 1 pyridoxine HCl co-crystal has an XRPD pattern comprising peaks at angles 2-theta of about 10.7±δx, 11.4±δx, 12.7±δx, and 14.8±δx degrees. In some embodiments, the Compound 1 pyridoxine HCl co-crystal has an XRPD pattern comprising peaks at angles 2-theta of about 10.3±δx, 10.7±δx, 10.9±δx, 11.4±δx, 12.7±δx, 14.8±δx, 15.6±δx, 17.0±δx, 17.5±δx, 17.9±δx, 18.7±δx, 19.7±δx, 20.0±δx, 20.3±δx, 20.8±δx, 21.3±δx, 21.6±δx, 21.8±δx, 22.6±δx, 22.8±δx, 23.3±δx, 24.2±δx, 24.8±δx, 25.0±δx, 25.8±δx, 26.5±δx, 26.9±δx, 27.5±δx, 27.8±δx, 28.2±δx, 28.7±δx, 29.8±δx, 30.2±δx, 30.8±δx, 31.5±δx, 32.0±δx, 32.2±δx, 32.9±δx, 33.8±δx, 34.4±δx, 35.3±δx, 35.5±δx, 37.0±δx, 37.7±δx, 38.1±δx, or 38.8±δx degrees, or any combination thereof. In some embodiments. δx is 0 degrees 2-theta In some embodiments, δx is 0.2 degrees 2-theta. In some embodiments, δx is 0.1, 0.2, 0.3, 0.4, 0.5, 0.6, 0.7, 0.8, 0.9, 1.0, 1.5, 2.0, 2.5, 3.0, 4.0, 5.0, 6.0, 7.0, 8.0, 9.0, or 10 degrees 2-theta.

In some embodiments, the Compound 1 pyridoxine HCl co-crystal has a differential scanning calorimetry (DSC) graph substantially as shown in FIG. 2B. In some embodiments, the Compound 1 pyridoxine HCl co-crystal is characterized by having a DSC graph comprising an endothermic peak at about 134.7° C. In some embodiments, the Compound 1 pyridoxine HCl co-crystal is characterized by having a DSC graph comprising an endothermic peak at 134.7° C.±δ_{DSC}, where δ_{DSC} is 0.1, 0.2, 0.3, 0.4, 0.5, 0.6, 0.7, 0.8, 0.9, 1, 1.25, 1.5, 1.75, 2, 2.5, 3, 3.5, 4, 4.5, 5, 6, 7, 8, 9, or 10° C. In some embodiments, δ_{DSC} is 2° C. In some embodiments, the Compound 1 pyridoxine HCl co-crystal is characterized by having a DSC graph comprising an endothermic peak between 133° C. and 137° C.

In some embodiments, the Compound 1 pyridoxine HCl co-crystal melts at or has a melting point at about 134.7° C. as determined by DSC. In some embodiments, the Compound 1 pyridoxine HCl co-crystal melts at or has a melting point at about 134.7° C.±δ_{DSC} as determined by DSC, where δ_{DSC} is 0.1, 0.2, 0.3, 0.4, 0.5, 0.6, 0.7, 0.8, 0.9, 1, 1.25, 1.5, 1.75, 2, 2.5, 3, 3.5, 4, 4.5, 5, 6, 7, 8, 9, or 10° C. In some embodiments, δ_{DSC} is 2° C. In some embodiments, the Compound 1 pyridoxine HCl co-crystal melts between about 133° C. and about 137° C. as determined by DSC.

In some embodiments, Compound 1 pyridoxine HCl co-crystal has a thermogravimetric analysis (TGA) graph substantially as shown in FIG. 2C. In some embodiments, Compound 1 pyridoxine HCl co-crystal is unsolvated as determined by TGA. In some embodiments, Compound 1 pyridoxine HCl co-crystal is anhydrous as determined by TGA. In some embodiments, % weight decrease of Compound 1 pyridoxine HCl co-crystal as determined by TGA from about 25° C. to about 130° C. is about 0.3%. In some embodiments, % weight decrease of Compound 1 pyridoxine HCl co-crystal as determined by TGA from about 25° C. to about 130° C. is about 0.1%, 0.2%, 0.3%, 0.4%, 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, or 10%. In some embodiments, % weight decrease of Compound 1 pyridoxine HCl co-crystal as determined by TGA from about 25° C. to about 130° C. is at least about 0.1%, 0.2%, 0.3%, 0.4%, 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, or 10%.

In some embodiments, Compound 1 pyridoxine HCl co-crystal has a dynamic vapor sorption (DVS) graph substantially as shown in FIG. 2D. In some embodiments, Compound 1 pyridoxine HCl co-crystal is hygroscopic. In some embodiments, Compound 1 pyridoxine HCl co-crystal is deliquescent. In some embodiments, Compound 1 pyridoxine HCl co-crystal undergoes a weight change of not more than about 0.6% at about 98% RH. In some embodiments, Compound 1 pyridoxine HCl co-crystal is reversibly hygroscopic. In some embodiments, Compound 1 pyridoxine HCl co-crystal does not undergo hysteresis. In some embodiments, Compound 1 pyridoxine HCl co-crystal is stable under various humidity conditions. In some embodiments, % weight increase of Compound 1 pyridoxine HCl co-crystal recovers to about 0% after a DVS cycle running from 5% RH to 95% RH. In some embodiments, crystalline form of Compound 1 pyridoxine HCl co-crystal remains unchanged after a DVS analysis running from 5% RH to 95% RH.

In some embodiments, % weight increase of Compound 1 pyridoxine HCl co-crystal as determined by DVS at about 95% RH is about 0.12%. In some embodiments. % weight increase of Compound 1 pyridoxine HCl co-crystal as determined by DVS from about 55% RH to about 85% RH is about 0.06% or at least 0.05%. In some embodiments, % weight increase of Compound 1 pyridoxine HCl co-crystal as determined by DVS from about 55% RH to about 85% RH is at least 0.01, 0.02, 0.03, 0.04, 0.05, 0.06, 0.07, 0.08, 0.09, 0.1, 0.15, 0.2, 0.25, 0.3, 0.35, 0.4, 0.45, 0.5, 1, 2, 3, 4, or 5%. In some embodiments, % weight increase of Compound 1 pyridoxine HCl co-crystal as determined by DVS from about 55% RH to about 85% RH is about 0.01, 0.02, 0.03, 0.04, 0.05, 0.06, 0.07, 0.08, 0.09, 0.1, 0.15, 0.2, 0.25, 0.3, 0.35, 0.4, 0.45, 0.5, 1, 2, 3, 4, or 5%.

In some embodiments, Compound 1 pyridoxine HCl co-crystal has an IR spectrum displaying at least one, at least two, at least three, at least four, at least five, at least six, at least seven, at least eight, at least nine, at least ten or more of the peaks in the IR spectrum substantially as shown in FIG. 2F or as provided in Table 2E.

In some embodiments, the Compound 1 pyridoxine HCl co-crystal has an IR spectrum displaying at least one, at least two, at least three, at least four, at least five, at least six, at least seven, at least eight, at least nine, at least ten or more of the peaks at angles 2-theta with the greatest intensity in the IR spectrum substantially as shown in FIG. 2F or as provided in Table 2E. It should be understood that relative intensities can vary depending on a number of factors, including sample preparation, mounting, and the instrument and analytical procedure and settings used to obtain the spectrum. Relative peak intensities and peak assignments can vary within experimental error. In some embodiments, peak assignments listed herein, including for the Compound 1 pyridoxine HCl co-crystal, can vary by about 0.1, ±0.2, ±0.3, ±0.4, ±0.5, ±0.6, ±0.7, ±0.8, ±0.9, ±0.10, ±0.15, ±0.2, ±0.25, ±0.3, ±0.35, ±0.4, ±0.45, ±0.5, ±0.75, ±1, ±1.25, ±1.5, ±2, ±2.5, ±3, ±3.5, ±4, ±4.5, ±5, ±6, ±7, ±8, ±9, or ±10 cm⁻¹.

In some embodiments, the Compound 1 pyridoxine HCl co-crystal has an IR spectrum comprising one or more peaks at about 409, 450, 475, 511, 575, 589, 622, 653, 685, 724, 749, 791, 841, 870, 926, 962, 989, 1017, 1089, 1115, 1191, 1214, 1253, 1275, 1307, 1339, 1368, 1382, 1400, 1413, 1439, 1447, 1460, 1480, 1545, 1567, 1587, 1625, 1645, 1679, 1732, 1805, 1826, 1900, 1929, 2817, 2955, 3093, 3232, 3267, or 3317 cm⁻¹, or any combination thereof. In some embodiments, the Compound 1 pyridoxine HCl co-crystal has an IR spectrum comprising peaks at about 575, 749, 791, 870, 1017, 1089, 1214, 1275, 1567, and 1679 cm⁻¹. In some embodiments, the Compound 1 pyridoxine HCl co-crystal has an IR spectrum comprising one or more peaks at about 409±δ_{IR}, 450±δ_{IR}, 475±δ_{IR}, 511±δ_{IR}, 575±δ_{IR}, 589±δ_{IR}, 622±δ_{IR}, 653±δ_{IR}, 685±δ_{IR}, 724±δ_{IR}, 749±δ_{IR}, 791±δ_{IR}, 841±δ_{IR}, 870±δ_{IR}, 926±δ_{IR}, 962±δ_{IR}, 989±δ_{IR}, 1017±δ_{IR}, 1089±δ_{IR}, 1115±δ_{IR}, 1191±δ_{IR}, 1214±δ_{IR}, 1253±δ_{IR}, 1275±δ_{IR}, 1307±δ_{IR}, 1339±δ_{IR}, 1368±δ_{IR}, 1382±δ_{IR}, 1400±δ_{IR}, 1413±δ_{IR}, 1439±δ_{IR}, 1447±δ_{IR}, 1460±δ_{IR}, 1480±δ_{IR}, 1545±δ_{IR}, 1567±δ_{IR}, 1587±δ_{IR}, 1625±δ_{IR}, 1645±δ_{IR}, 1679±δ_{IR}, 1732±δ_{IR}, 1805±δ_{IR}, 1826±δ_{IR}, 1900±δ_{IR}, 1929±δ_{IR}, 2817±δ_{IR}, 2955±δ_{IR}, 3093±δ_{IR}, 3232±δ_{IR}, 3267±δ_{IR}, or 3317±δ_{IR} cm⁻¹, or any combination thereof. In some embodiments, the Compound 1 pyridoxine HCl co-crystal has an IR spectrum comprising peaks at about 575±δ_{IR}, 749±δ_{IR}, 791±δ_{IR}, 870±δ_{IR}, 1017±δ_{IR}, 1089±δ_{IR}, 1214±δ_{IR}, 1275±δ_{IR}, 1567±δ_{IR}, and 1679±δ_{IR} cm⁻¹. In some embodiments, δ_{IR} is 0, 1, 0.2, 0.3, 0.4, 0.5, 0.6, 0.7, 0.8, 0.9, 0.10, 0.15, 0.2, 0.25, 0.3, 0.35, 0.4, 0.45, 0.5, 0.75, 1, 1.25, 1.5, 2, 2.5, 3, 3.5, 4, 4.5, 5, 6, 7, 8, 9, or 10 cm⁻¹. In some embodiments, δ_{IR} is 2 cm⁻¹.

In some embodiments, Compound 1 pyridoxine HCl co-crystal has an FT-Raman spectrum displaying at least one, at least two, at least three, at least four, at least five, at least six, at least seven, at least eight, at least nine, at least ten or more of the peaks in the FT-Raman spectrum substantially as shown in FIG. 2G or as provided in Table 2F.

In some embodiments, the Compound 1 and pyridoxine HCl has an FT-Raman spectrum displaying at least one, at least two, at least three, at least four, at least five, at least six, at least seven, at least eight, at least nine, at least ten or more of the peaks at angles 2-theta with the greatest intensity in the FT-Raman spectrum substantially as shown in FIG. 2G or as provided in Table 2F. It should be understood that relative intensities can vary depending on a number of factors, including sample preparation, mounting, and the instrument and analytical procedure and settings used to obtain the spectrum. Relative peak intensities and peak assignments can vary within experimental error. In some embodiments, peak assignments listed herein, including for the Compound 1 pyridoxine HCl co-crystal, can vary by about ±0.1, ±0.2, ±0.3, ±0.4, ±0.5, ±0.6, ±0.7, ±0.8, 10.9, ±0.10, ±0.15, ±0.2, ±0.25, 10.3, ±0.35, ±0.4, ±0.45, ±0.5, ±0.75, ±1, ±1.25, ±1.5, ±2, ±2.5, ±3, ±3.5, ±4, ±4.5, ±5, ±6, ±7, ±8, ±9, or ±10 cm⁻¹.

In some embodiments, the Compound 1 pyridoxine HCl co-crystal has an FT-Raman spectrum comprising one or more peaks at about 116, 161, 257, 286, 309, 329, 356, 404, 477, 517, 533, 568, 654, 691, 724, 753, 791, 841, 868, 897, 928, 965, 988, 1009, 1052, 1088, 1123, 1216, 1233, 1293, 1322, 1356, 1356, 1370, 1381, 1449, 1569, 1588, 1628, 1644, 1678, 2826, 2868, 2901, 2934, 2959, 2999, 3015, or 3102 cm⁻¹, or any combination thereof. In some embodiments, the Compound 1 pyridoxine HCl co-crystal has an FT-Raman spectrum comprising peaks at about 329, 404, 691, 753, 791, 897, 1233, 1449, 1644, and 1678 cm⁻¹. In some embodiments, the Compound 1 pyridoxine HCl co-crystal has an FT-Raman spectrum comprising one or more peaks at about 116±δ_{FT}, 161±δ_{FT}, 257±δ_{FT}, 286±δ_{FT}, 309±δ_{FT}, 329±δ_{FT}, 356±δ_{FT}, 404±δ_{FT}, 477±δ_{FT}, 517±δ_{FT}, 533±δ_{FT}, 568±δ_{FT}, 654±δ_{FT}, 691±δ_{FT}, 724±δ_{FT}, 753±δ_{FT}, 791±δ_{FT}, 841±δ_{FT}, 868±δ_{FT}, 897±δ_{FT}, 928±δ_{FT}, 965±δ_{FT}, 988±δ_{FT}, 1909±δ_{FT}, 1052±δ_{FT}, 1088±δ_{FT}, 1123±δ_{FT}, 1216±δ_{FT}, 1233±δ_{FT}, 1293±δ_{FT}, 1322±δ_{FT}, 1356±δ_{FT}, 1356±δ_{FT}, 1370±δ_{FT}, 1381±δ_{FT}, 1449±δ_{FT}, 1569±δ_{FT}, 1588±δ_{FT}, 1628±δ_{FT}, 1644±δ_{FT}, 1678±δ_{FT}, 2826±δ_{FT}, 2868±δ_{FT}, 2901±δ_{FT}, 2934±δ_{FT}, 2959±δ_{FT}, 2999±δ_{FT}, 3015±δ_{FT}, or 3102±δ_{FT} cm⁻¹, or any combination thereof. In some embodiments, the Compound 1 pyridoxine HCl co-crystal has an FT-Raman spectrum comprising peaks at about 329±δ_{FT}, 404±δ_{FT}, 691±δ_{FT}, 753±δ_{FT}, 791±δ_{FT}, 897±δ_{FT}, 1233±δ_{FT}, 1449±δ_{FT}, 1644±δ_{FT}, and 1678±δ_{FT} cm⁻¹. In some embodiments, δ_{FT} is 0, 1, 0.2, 0.3, 0.4, 0.5, 0.6, 0.7, 0.8, 0.9, 0.10, 0.15, 0.2, 0.25, 0.3, 0.35, 0.4, 0.45, 0.5, 0.75, 1, 1.25, 1.5, 2, 2.5, 3, 3.5, 4, 4.5, 5, 6, 7, 8, 9, or 10 cm⁻¹. In some embodiments, δ_{FT} is 2 cm⁻¹.

In some embodiments, Compound 1 pyridoxine HCl co-crystal has an LF-Raman spectrum displaying at least one, at least two, at least three, at least four, at least five, at least six, at least seven, at least eight, at least nine, at least ten or more of the peaks in the LF-Raman spectrum substantially as shown in FIG. 2H or as provided in Table 2H.

In some embodiments, the Compound 1 pyridoxine HCl co-crystal has an LF-Raman spectrum displaying at least one, at least two, at least three, at least four, at least five, at least six, at least seven, at least eight, at least nine, at least ten or more of the peaks at angles 2-theta with the greatest intensity in the LF-Raman spectrum substantially as shown in FIG. 2H or as provided in Table 2H. It should be understood that relative intensities can vary depending on a number of factors, including sample preparation, mounting, and the instrument and analytical procedure and settings used to obtain the spectrum. Relative peak intensities and peak assignments can vary within experimental error. In some embodiments, peak assignments listed herein, including for the Compound 1 pyridoxine HCl co-crystal, can vary by about ±0.1, 40.2, ±0.3, ±0.4, ±0.5, 10.6, ±0.7, ±0.8, ±0.9, ±0.10, ±0.15, ±0.2, ±0.25, ±0.3, ±0.35, ±0.4, ±0.45, ±0.5, ±0.75, ±1, ±1.25, ±1.5, ±2, ±2.5, ±3, ±3.5, ±4, ±4.5, ±5, ±6, ±7, ±8, ±9, or ±10 cm⁻¹.

In some embodiments, the Compound 1 pyridoxine HCl co-crystal has an LF-Raman spectrum comprising one or more peaks at about −329, −285, −167, −118, −83, or −57 cm⁻¹, or any combination thereof. In some embodiments, the Compound 1 pyridoxine HCl co-crystal has an LF-Raman spectrum comprising peaks at about −329, −118, −83, and −57 cm⁻¹. In some embodiments, the Compound 1 pyridoxine HCl co-crystal has an LF-Raman spectrum comprising one or more peaks at about −329±δ_{LF}, −285±δ_{LF}, −167±δ_{LF}, −118±δ_{LF}, −83±δ_{LF}, or −57±δ_{LF} cm⁻¹, or any combination thereof. In some embodiments, the Compound 1 pyridoxine HCl co-crystal has an LF-Raman spectrum comprising peaks at about −329±δ_{LF}, −118±δ_{LF}, −83±δ_{LF}, and −57±δ_{LF} cm⁻¹. In some embodiments, δ_{LF} is 0.1, 0.2, 0.3, 0.4, 0.5, 0.6, 0.7, 0.8, 0.9, 0.10, 0.15, 0.2, 0.25, 0.3, 0.35, 0.4, 0.45, 0.5, 0.75, 1, 1.25, 1.5, 2, 2.5, 3, 3.5, 4, 4.5, 5, 6, 7, 8, 9, or 10 cm⁻¹. In some embodiments, δ_{LF} is 2 cm⁻¹.

In some embodiments, Compound 1 pyridoxine HCl co-crystal has a ¹H NMR spectrum substantially as shown in FIG. 2I. In some embodiments, the Compound 1 pyridoxine HCl co-crystal has ¹H NMR spectrum comprising one or more peaks at about 2.56, 4.07, 4.29, 4.71, 4.80, 4.84, 5.16, or 8.16 ppm, or any combination thereof. In some embodiments, the Compound 1 pyridoxine HCl co-crystal has an ¹H NMR spectrum comprising one or more peaks at 2.56±δ_{N}, 4.07±δ_{N}, 4.29±δ_{N}, 4.71±δ_{N}, 4.80±δ_{N}, 4.84±δ_{N}, 5.16±δ_{N}, or 8.16±δ_{N} ppm, or any combination thereof. In some embodiments, δ_{N} is 0.01, 0.05, 0.1, 0.2, 0.3, 0.4, 0.5, 0.6, 0.7, 0.8, 0.9, 0.10, 0.15, 0.2, 0.25, 0.3, 0.35, 0.4, 0.45, 0.5, 0.75, 1, 1.25, 1.5, 2, 3, 4, or 5 ppm. In some embodiments, δ_{N} is 0.2 ppm.

In some embodiments, Compound 1 pyridoxine HCl co-crystal is crystalline. In some embodiments, Compound 1 pyridoxine HCl co-crystal is unsolvated. In some embodiments, Compound 1 pyridoxine HCl co-crystal is anhydrous. In some embodiments, Compound 1 pyridoxine HCl co-crystal has a 1:1 stoichiometry. In some embodiments, Compound 1 pyridoxine HCl co-crystal is not impact-sensitive. In some embodiments, Compound 1 pyridoxine HCl co-crystal is less impact-sensitive than an Compound 1 crystal.

In some embodiments, the solubility of Compound 1 pyridoxine HCl co-crystal in water or in a mixture of water with other solvents is higher than the solubility of an amorphous or crystalline form of Compound 1. In some embodiments, the solubility of Compound 1 pyridoxine HCl co-crystal herein may be significantly higher than the solubility of an amorphous or crystalline form of Compound 1. In some embodiments, the solubility of Compound 1 pyridoxine HCl co-crystal is at least about 1%, 5%, 10%, 15%, 20%, 30%, 40%, 50%, 60%, 70%, 80%, 90%, 100%, 150%, 200%, 250%, 300%, 400%, 500%, 600%, 700%, 800%, or 900% higher than the solubility of an amorphous or crystalline form of Compound 1. In some embodiments, solubility is measured as grams of Compound 1 pyridoxine HCl co-crystal soluble in 100 g water.

### 3. Compound 1 Thiamine HCl Co-crystal

In one aspect, provided herein is a co-crystal of Compound 1 and thiamine HCl.

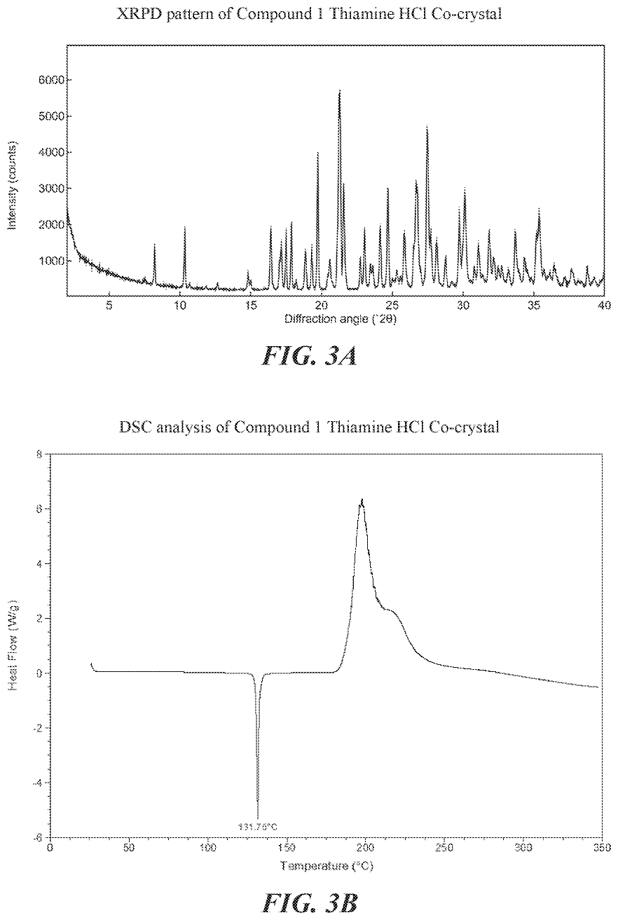

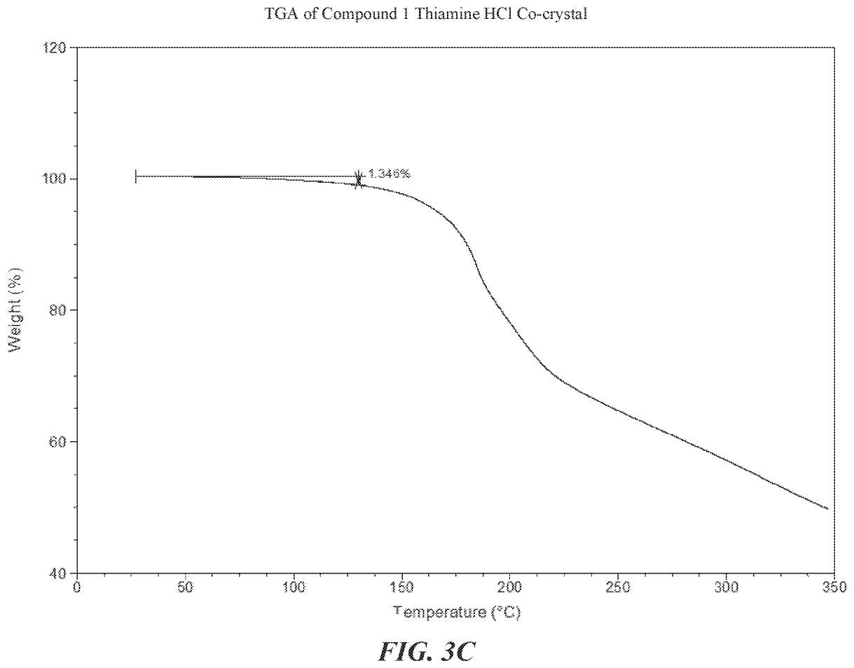

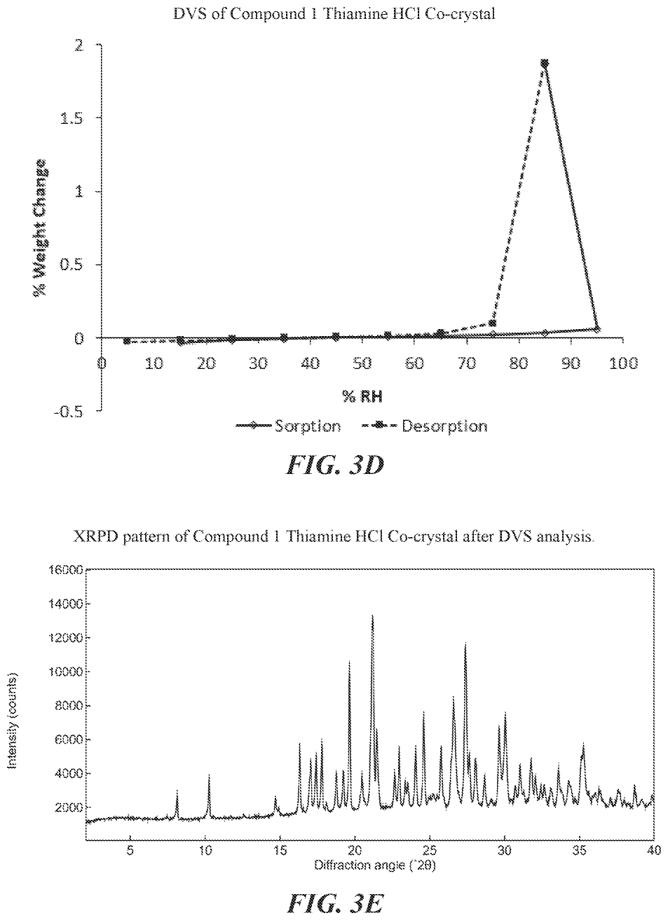

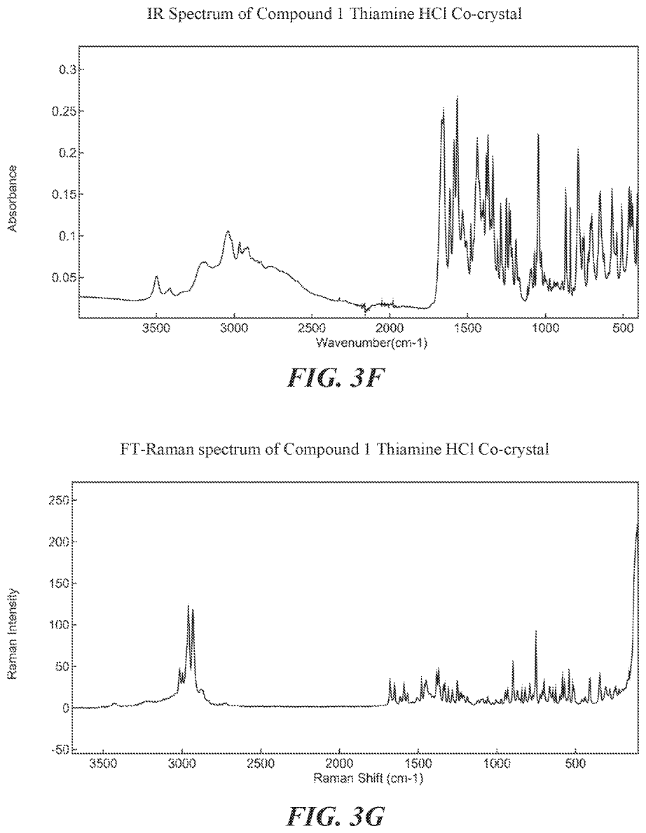

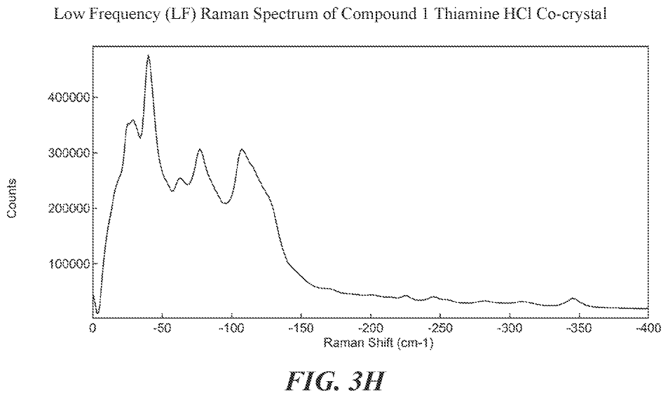

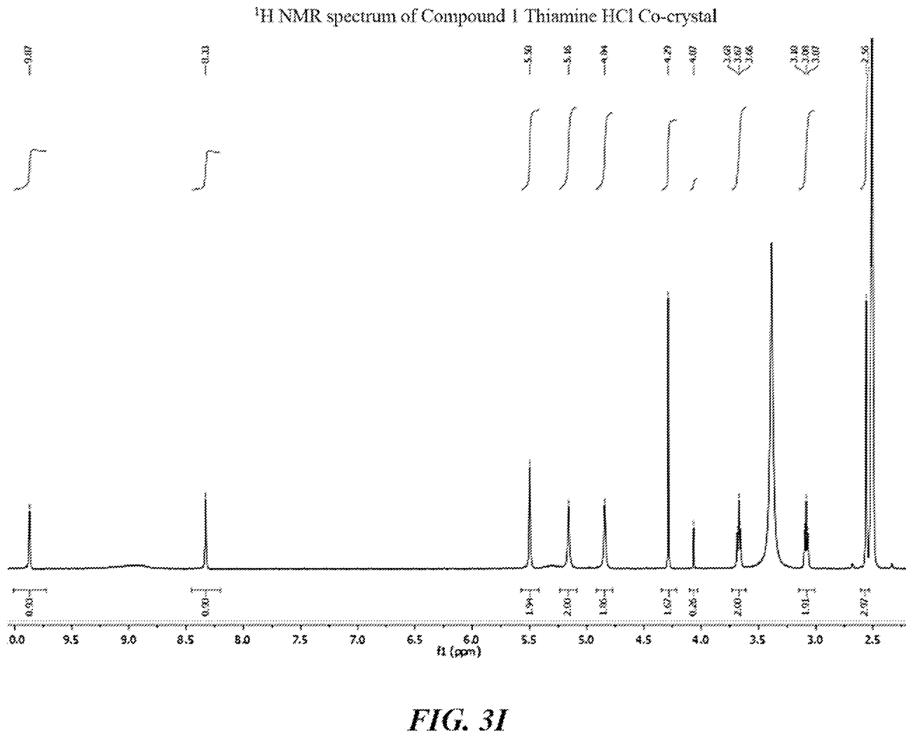

In some embodiments, the Compound 1 thiamine HCl co-crystal has an XRPD pattern displaying at least one, at least two, at least three, at least four, at least five, at least six, at least seven, at least eight, at least nine, at least ten or more of the peaks at angles 2-theta in the XRPD pattern substantially as shown in FIG. 3A or as provided in Table 3B. In some embodiments, the Compound 1 thiamine HCl co-crystal has an XRPD pattern displaying at least one, at least two, at least three, at least four, at least five, at least six, at least seven, at least eight, at least nine, at least ten or more of the peaks at angles 2-theta with the greatest intensity in the XRPD pattern substantially as shown in FIG. 3A or as provided in Table 3B. It should be understood that relative intensities can vary depending on a number of factors, including sample preparation, mounting, and the instrument and analytical procedure and settings used to obtain the spectrum. Relative peak intensities and peak assignments can vary within experimental error. In some embodiments, peak assignments listed herein, including for the Compound 1 thiamine HCl co-crystal, can vary by about ±2.0 degrees, ±1.5 degrees, ±1.0 degrees, ±0.9 degrees, ±0.8 degrees, ±0.7 degrees, ±0.6 degrees, ±0.5 degrees, ±0.4 degrees, ±0.3 degrees, ±0.2 degrees, or ±0.1 degrees 2-theta.

In some embodiments, the Compound 1 thiamine HCl co-crystal has an XRPD pattern comprising one or more peaks at angles 2-theta of about 8.2±δx, 10.3±δx, and 16.4±δx degrees. In some embodiments, the Compound 1 thiamine HCl co-crystal has an XRPD pattern comprising peaks at angles 2-theta of about 7.5±δx, 8.2±δx, 10.3±δx, 14.8±δx and 16.4±δx degrees. In some embodiments, the Compound 1 thiamine HCl co-crystal has an XRPD pattern comprising peaks at angles 2-theta of about 7.5±δx, 8.2±δx, 10.3±δx, 10.7±δx, 11.9±δx, 12.7±δx, 14.8±δx, 15.0±δx, 16.4±δx, 17.2±δx, 17.5±δx, 17.9±δx, 18.2±δx, 18.9±δx, 19.3±δx, 19.7±δx, 20.6±δx, 21.3±δx, 21.6±δx, 22.7±δx, 23.0±δx, 23.5±δx, 23.6±δx, 24.1±δx, 24.7±δx, 25.0±δx, 25.3±δx, 25.6±δx, 25.9±δx, 26.7±δx, 27.5±δx, 27.7±δx, 28.1±δx, 28.8±δx, 29.2±δx, 29.7±δx, 30.1±δx, 30.8±δx, 31.1±δx, 31.8±δx, 32.1±δx, 32.5±δx, 32.7±δx, 33.2±δx, 33.7±δx, 34.3±δx, 34.8±δx, 35.4±δx, 35.7±δx, 36.1±δx, 36.4±δx, 37.2±δx, 37.7±δx, 38.8±δx, or 39.2±δx degrees, or any combination thereof. In some embodiments, δx is 0 degrees 2-theta. In some embodiments, δx is 0.2 degrees 2-theta. In some embodiments, δx is 0.1, 0.2, 0.3, 0.4, 0.5, 0.6, 0.7, 0.8, 0.9, 1.0, 1.5, 2.0, 2.5, 3.0, 4.0, 5.0, 6.0, 7.0, 8.0, 9.0, or 10 degrees 2-theta.

In some embodiments, the Compound 1 thiamine HCl co-crystal has a differential scanning calorimetry (DSC) graph substantially as shown in FIG. 3B. In some embodiments, the Compound 1 thiamine HCl co-crystal is characterized by having a DSC graph comprising an endothermic peak at about 131.8° C. In some embodiments, the Compound 1 thiamine HCl co-crystal is characterized by having a DSC graph comprising an endothermic peak at 131.8° C.±δ_{DSC}, where δ_{DSC} is 0.1, 0.2, 0.3, 0.4, 0.5, 0.6, 0.7, 0.8, 0.9, 1, 1.25, 1.5, 1.75, 2, 2.5, 3, 3.5, 4, 4.5, 5, 6, 7, 8, 9, or 10° C. In some embodiments, δ_{DSC} is 2° C. In some embodiments, the Compound 1 thiamine HCl co-crystal is characterized by having a DSC graph comprising an endothermic peak between about 131° C. and about 134° C.

In some embodiments, the Compound 1 thiamine HCl co-crystal melts at or has a melting point at about 131.8° C. as determined by DSC. In some embodiments, the Compound 1 thiamine HCl co-crystal melts at or has a melting point at about 131.8° C.±δ_{DSC} as determined by DSC, where δ_{DSC} is 0.1, 0.2, 0.3, 0.4, 0.5, 0.6, 0.7, 0.8, 0.9, 1, 1.25, 1.5, 1.75, 2, 2.5, 3, 3.5, 4, 4.5, 5, 6, 7, 8, 9, or 10° C. In some embodiments, δ_{DSC} is 2° C. In some embodiments, the Compound 1 thiamine HCl co-crystal melts between about 131° C. and about 134° C. as determined by DSC.

In some embodiments, Compound 1 thiamine HCl co-crystal has a thermogravimetric analysis (TGA) graph substantially as shown in FIG. 3C. In some embodiments, Compound 1 thiamine HCl co-crystal is a monohydrate as determined by TGA. In some embodiments, % weight decrease of Compound 1 thiamine HCl co-crystal as determined by TGA from about 25° C. to about 130° C. is about 1.3%. In some embodiments, % weight decrease of Compound 1 thiamine HCl co-crystal as determined by TGA from about 25° C. to about 130° C. is about 0.1%, 0.2%, 0.3%, 0.4%, 0.5%, 1%, 1.1%, 1.2%, 1.3%, 1.4%, 1.5%, 1.6%, 1.7%, 1.8%, 1.9%, 2%, 2.5%, 3%, 3.5%, 4%, 4.5%, 5%, 5.5%, 6%, 6.5%, 7%, 7.5%, 8%, 8.5%, 9%, 9.5%, or 10%. In some embodiments, % weight decrease of Compound 1 thiamine HCl co-crystal as determined by TGA from about 25° C. to about 130° C. is at least about 0.1%, 0.2%, 0.3%, 0.4%, 0.5%, 1%, 1.1%, 1.2%, 1.3%, 1.4%, 1.5%, 1.6%, 1.7%, 1.8%, 1.9%, 2%, 2.5%, 3%, 3.5%, 4%, 4.5%, 5%, 5.5%, 6%, 6.5%, 7%, 7.5%, 8%, 8.5%, 9%, 9.5%, or 10%.

In some embodiments, Compound 1 thiamine HCl co-crystal has a dynamic vapor sorption (DVS) graph substantially as shown in FIG. 3D. In some embodiments, Compound 1 thiamine HCl co-crystal is hygroscopic. In some embodiments, Compound 1 thiamine HCl co-crystal is deliquescent. In some embodiments, Compound 1 thiamine HCl co-crystal undergoes a weight change of not more than about 2% at about 89% RH. In some embodiments, Compound 1 thiamine HCl co-crystal is reversibly hygroscopic. In some embodiments, Compound 1 thiamine HCl co-crystal does not undergo hysteresis. In some embodiments, Compound 1 thiamine HCl co-crystal is stable under various humidity conditions. In some embodiments, % weight increase of Compound 1 thiamine HCl co-crystal recovers to about 0% after a DVS cycle running from 5% RH to 95% RH. In some embodiments, crystalline form of Compound 1 thiamine HCl co-crystal remains unchanged after a DVS analysis running from 5% RH to 95% RH.

In some embodiments, % weight increase of Compound 1 thiamine HCl co-crystal as determined by DVS at about 95% RH is about 0.06%. In some embodiments, % weight increase of Compound 1 thiamine HCl co-crystal as determined by DVS from about 25% RH to about 95% RH is about 0.05% or at least 0.05%. In some embodiments, % weight increase of Compound 1 thiamine HCl co-crystal as determined by DVS from about 25% RH to about 95% RH is at least 0.01, 0.02, 0.03, 0.04, 0.05, 0.06, 0.07, 0.08, 0.09, 0.1, 0.15, 0.2, 0.25, 0.3, 0.35, 0.4, 0.45, 0.5, 1, 2, 3, 4, or 5%. In some embodiments, % weight increase of Compound 1 thiamine HCl co-crystal as determined by DVS from about 55% RH to about 85% RH is about 0.01, 0.02, 0.03, 0.04, 0.05, 0.06, 0.07, 0.08, 0.09, 0.1, 0.15, 0.2, 0.25, 0.3, 0.35, 0.4, 0.45, 0.5, 1, 2, 3, 4, or 5%.

In some embodiments, Compound 1 thiamine HCl co-crystal has an IR spectrum displaying at least one, at least two, at least three, at least four, at least five, at least six, at least seven, at least eight, at least nine, at least ten or more of the peaks in the IR spectrum substantially as shown in FIG. 3F or as provided in Table 3E.

In some embodiments, the Compound 1 thiamine HCl co-crystal has an IR spectrum displaying at least one, at least two, at least three, at least four, at least five, at least six, at least seven, at least eight, at least nine, at least ten or more of the peaks at angles 2-theta with the greatest intensity in the IR spectrum substantially as shown in FIG. 3F or as provided in Table 3E. It should be understood that relative intensities can vary depending on a number of factors, including sample preparation, mounting, and the instrument and analytical procedure and settings used to obtain the spectrum. Relative peak intensities and peak assignments can vary within experimental error. In some embodiments, peak assignments listed herein, including for the Compound 1 thiamine HCl co-crystal, can vary by about ±0.1, ±0.2, ±0.3, ±0.4, ±0.5, ±0.6, ±0.7, ±0.8, ±0.9, ±0.10, ±0.15, ±0.2, ±0.25, ±0.3, ±0.35, ±0.4, ±0.45, ±0.5, ±0.75, ±1, ±1.25, ±1.5, ±2, ±2.5, ±3, ±3.5, ±4, ±4.5, ±5, ±6, ±7, ±8, ±9, or ±10 cm⁻¹.

In some embodiments, the Compound 1 thiamine HCl co-crystal has an IR spectrum comprising one or more peaks at about 408, 440, 450, 463, 509, 542, 572, 645, 702, 724, 751, 760, 790, 841, 870, 891, 972, 1007, 1026, 1045, 1072, 1095, 1115, 1170, 1189, 1213, 1225, 1233, 1251, 1286, 1308, 1339, 1368, 1380, 1399, 1438, 1480, 1508, 1532, 1567, 1587, 1613, 1655, 2915, 2964, 3038, 3193, 3414, or 3498 cm⁻¹, or any combination thereof. In some embodiments, the Compound 1 thiamine HCl co-crystal has an IR spectrum comprising peaks at about 509, 572, 645, 702, 790, 841, 870, 1045, 1339, 1368, 1438, 1567, and 1655 cm⁻¹. In some embodiments, the Compound 1 thiamine HCl co-crystal has an IR spectrum comprising one or more peaks at about 408±δ_{IR}, 440±δ_{IR}, 450±δ_{IR}, 463±δ_{IR}, 509±δ_{IR}, 542±δ_{IR}, 572±δ_{IR}, 645±δ_{IR}, 702±δ_{IR}, 724±δ_{IR}, 751±δ_{IR}, 760±δ_{IR}, 790±δ_{IR}, 841±δ_{IR}, 87±δ_{IR}, 891±δ_{IR}, 972±δ_{IR}, 1007±δ_{IR}, 1026±δ_{IR}, 1045±δ_{IR}, 1072±δ_{IR}, 1095±δ_{IR}, 1115±δ_{IR}, 1170±δ_{IR}, 1189±δ_{IR}, 1213±δ_{IR}, 1225±δ_{IR}, 1233±δ_{IR}, 1251±δ_{IR}, 1286±δ_{IR}, 1308±δ_{IR}, 1339±δ_{IR}, 1368±δ_{IR}, 1380±δ_{IR}, 1399±δ_{IR}, 1438±δ_{IR}, 1480±δ_{IR}, 1508±δ_{IR}, 1532±δ_{IR}, 1567±δ_{IR}, 1587±δ_{IR}, 1613±δ_{IR}, 1655±δ_{IR}, 2915±δ_{IR}, 2964±δ_{IR}, 3038±δ_{IR}, 3193±δ_{IR}, 3414±δ_{IR}, or 3498±δ_{IR} cm⁻¹, or any combination thereof. In some embodiments, the Compound 1 thiamine HCl co-crystal has an IR spectrum comprising peaks at about 509±δ_{IR}, 572±δ_{IR}, 645±δ_{IR}, 702±δ_{IR}, 790±δ_{IR}, 841±δ_{IR}, 870±δ_{IR}, 1045±δ_{IR}, 1339±δ_{IR}, 1368±δ_{IR}, 1438±δ_{IR}, 1567±δ_{IR}, and 1655±δ_{IR} cm⁻¹. In some embodiments. δ_{IR} is 0.1, 0.2, 0.3, 0.4, 0.5, 0.6, 0.7, 0.8, 0.9, 0.10, 0.15, 0.2, 0.25, 0.3, 0.35, 0.4, 0.45, 0.5, 0.75, 1, 1.25, 1.5, 2, 2.5, 3, 3.5, 4, 4.5, 5, 6, 7, 8, 9, or 10 cm⁻¹. In some embodiments, δ_{IR} is 2 cm⁻¹.

In some embodiments, Compound 1 thiamine HCl co-crystal has an FT-Raman spectrum displaying at least one, at least two, at least three, at least four, at least five, at least six, at least seven, at least eight, at least nine, at least ten or more of the peaks in the FT-Raman spectrum substantially as shown in FIG. 3G or as provided in Table 3F.

In some embodiments, the Compound 1 thiamine HCl co-crystal has an FT-Raman spectrum displaying at least one, at least two, at least three, at least four, at least five, at least six, at least seven, at least eight, at least nine, at least ten or more of the peaks at angles 2-theta with the greatest intensity in the FT-Raman spectrum substantially as shown in FIG. 3G or as provided in Table 3F. It should be understood that relative intensities can vary depending on a number of factors, including sample preparation, mounting, and the instrument and analytical procedure and settings used to obtain the spectrum. Relative peak intensities and peak assignments can vary within experimental error. In some embodiments, peak assignments listed herein, including for the Compound 1 thiamine HCl co-crystal, can vary by about ±0.1, ±0.2, ±0.3, ±0.4, ±0.5, ±0.6, ±0.7, ±0.8, ±0.9, ±0.10, ±0.15, ±0.2, ±0.25, ±0.3, ±0.35, ±0.4, ±0.45, ±0.5, ±0.75, ±1, ±1.25, ±1.5, ±2, ±2.5, ±3, ±3.5, ±4, ±4.5, ±δ, ±6, ±7, ±8, ±9, or ±10 cm⁻¹.

In some embodiments, the Compound 1 thiamine HCl co-crystal has an FT-Raman spectrum comprising one or more peaks at about 109, 246, 282, 309, 346, 409, 517, 543, 571, 583, 627, 645, 666, 701, 751, 791, 820, 841, 868, 897, 932, 946, 973, 1007, 1057, 1189, 1212, 1235, 1250, 1283, 1308, 1331, 1370, 1381, 1449, 1480, 1507, 1569, 1590, 1613, 1651, 1678, 2882, 2932, 2959, 2998, or 3015 cm⁻¹, or any combination thereof. In some embodiments, the Compound 1 thiamine HCl co-crystal has an FT-Raman spectrum comprising peaks at about 346, 409, 751, 897, 1651, and 1679 cm⁻¹. In some embodiments, the Compound 1 thiamine HCl co-crystal has an FT-Raman spectrum comprising one or more peaks at about 109±δ_{FT}, 246±δ_{FT}, 282±δ_{FT}, 309±δ_{FT}, 346±δ_{FT}, 409±δ_{FT}, 517±δ_{FT}, 543±δ_{FT}, 571±δ_{FT}, 583±δ_{FT}, 627±δ_{FT}, 645±δ_{FT}, 666±δ_{FT}, 701±δ_{FT}, 751±δ_{FT}, 791±δ_{FT}, 820±δ_{FT}, 841±δ_{FT}, 868±δ_{FT}, 897±δ_{FT}, 932±δ_{FT}, 946±δ_{FT}, 973±δ_{FT}, 1907±δ_{FT}, 1057±δ_{FT}, 1189±δ_{FT}, 1212±δ_{FT}, 1235±δ_{FT}, 1250±δ_{FT}, 1283±δ_{FT}, 1308±δ_{FT}, 1331±δ_{FT}, 1370±δ_{FT}, 1381±δ_{FT}, 1449±δ_{FT}, 1480±δ_{FT}, 1507±δ_{FT}, 1569±δ_{FT}, 1590±δ_{FT}, 1613±δ_{FT}, 1651±δ_{FT}, 1678±δ_{FT}, 2882±δ_{FT}, 2932&δ_{FT}, 2959±δ_{FT}, 2998±δ_{FT}, or 3015±δ_{FT} cm⁻¹, or any combination thereof. In some embodiments, the Compound 1 thiamine HCl co-crystal has an FT-Raman spectrum comprising peaks at about 346±δ^{FT}, 409±δ_{FT}, 751±δ_{FT}, 897±δ_{FT}, 1651±δ_{FT}, and 1679±δ_{FT} cm⁻¹. In some embodiments, δ_{FT} is 0.1, 0.2, 0.3, 0.4, 0.5, 0.6, 0.7, 0.8, 0.9, 0.10, 0.15, 0.2, 0.25, 0.3, 0.35, 0.4, 0.45, 0.5, 0.75, 1, 1.25, 1.5, 2, 2.5, 3, 3.5, 4, 4.5, 5, 6, 7, 8, 9, or 10 cm⁻¹. In some embodiments, δ_{FT} is 2 cm⁻¹.

In some embodiments, Compound 1 thiamine HCl co-crystal has an LF-Raman spectrum displaying at least one, at least two, at least three, at least four, at least five, at least six, at least seven, at least eight, at least nine, at least ten or more of the peaks in the LF-Raman spectrum substantially as shown in FIG. 3H or as provided in Table 3H.

In some embodiments, the Compound 1 thiamine HCl co-crystal has an LF-Raman spectrum displaying at least one, at least two, at least three, at least four, at least five, at least six, at least seven, at least eight, at least nine, at least ten or more of the peaks at angles 2-theta with the greatest intensity in the LF-Raman spectrum substantially as shown in FIG. 3H or as provided in Table 3H. It should be understood that relative intensities can vary depending on a number of factors, including sample preparation, mounting, and the instrument and analytical procedure and settings used to obtain the spectrum. Relative peak intensities and peak assignments can vary within experimental error. In some embodiments, peak assignments listed herein, including for the Compound 1 thiamine HCl co-crystal, can vary by about ±0.1, ±0.2, ±0.3, ±0.4, ±0.5, ±0.6, ±0.7, ±0.8, ±0.9, ±0.10, ±0.15, ±0.2, ±0.25, ±0.3, ±0.35, ±0.4, ±0.45, ±0.5, ±0.75, ±1, ±1.25, ±1.5, ±2, ±2.5, ±3, ±3.5, ±4, ±4.5, ±δ, ±6, ±7, ±8, ±9, or ±10 cm⁻¹.

In some embodiments, the Compound 1 thiamine HCl co-crystal has an LF-Raman spectrum comprising one or more peaks at about −346, −107, −77, −62, −40, or −29 cm⁻¹, or any combination thereof. In some embodiments, the Compound 1 thiamine HCl co-crystal has an LF-Raman spectrum comprising peaks at about −107, −77, and −40 cm⁻¹. In some embodiments, the Compound 1 thiamine HCl co-crystal has an LF-Raman spectrum comprising one or more peaks at about −346±δ_{LF}, −107±δ_{LF}, −77±δ_{LF}, −62±δ_{LF}, −40±δ_{LF}, or −29±δ_{LF} cm⁻¹, or any combination thereof. In some embodiments, the Compound 1 thiamine HCl co-crystal has an LF-Raman spectrum comprising peaks at about −107±δ_{LF}, −77±δ_{LF}, and −40±δ_{LF} cm⁻¹. In some embodiments, δ_{LF} is 0.1, 0.2, 0.3, 0.4, 0.5, 0.6, 0.7, 0.8, 0.9, 0.10, 0.15, 0.2, 0.25, 0.3, 0.35, 0.4, 0.45, 0.5, 0.75, 1, 1.25, 1.5, 2, 2.5, 3, 3.5, 4, 4.5, 5, 6, 7, 8, 9, or 10 cm⁻¹. In some embodiments, δ_{LF} is 2 cm⁻¹.

In some embodiments, Compound 1 thiamine HCl co-crystal has a ¹H NMR spectrum substantially as shown in FIG. 3I. In some embodiments, the Compound 1 thiamine HCl co-crystal has ¹H NMR spectrum comprising one or more peaks at about 2.56, 3.07, 3.08, 3.10, 3.66, 3.67, 3.68, 4.07, 4.29, 4.84, 5.16, or 5.50 ppm, or any combination thereof. In some embodiments, the Compound 1 thiamine HCl co-crystal has an ¹H NMR spectrum comprising one or more peaks at 2.56±δ_{N}, 3.07±δ_{N}, 3.08±δ_{N}, 3.10±δ_{N}, 3.66±δ_{N}, 3.67±δ_{N}, 3.68±δ_{N}, 4.07±δ_{N}, 4.29±δ_{N}, 4.84±δ_{N}, 5.16±δ_{N}, or 5.50±δ_{N} ppm, or any combination thereof. In some embodiments, δ_{N} is 0.01, 0.05, 0.1, 0.2, 0.3, 0.4, 0.5, 0.6, 0.7, 0.8, 0.9, 0.10, 0.15, 0.2, 0.25, 0.3, 0.35, 0.4, 0.45, 0.5, 0.75, 1, 1.25, 1.5, 2, 3, 4, or 5 ppm. In some embodiments, δ_{N} is 0.2 ppm.

In some embodiments, Compound 1 thiamine HCl co-crystal is crystalline. In some embodiments, Compound 1 thiamine HCl co-crystal is unsolvated. In some embodiments, Compound 1 thiamine HCl co-crystal is monohydrate. In some embodiments, Compound 1 thiamine HCl co-crystal has a 1:1 stoichiometry. In some embodiments, Compound 1 thiamine HCl co-crystal is not impact-sensitive. In some embodiments, Compound 1 thiamine HCl co-crystal is less impact-sensitive than an Compound 1 crystal.

In some embodiments, the solubility of Compound 1 thiamine HCl co-crystal in water or in a mixture of water with other solvents is higher than the solubility of an amorphous or crystalline form of Compound 1. In some embodiments, the solubility of Compound 1 thiamine HCl co-crystal herein may be significantly higher than the solubility of an amorphous or crystalline form of Compound 1. In some embodiments, the solubility of Compound 1 thiamine HCl co-crystal is at least about 1%, 5%, 10%, 15%, 20%, 30%, 40%, 50%, 60%, 70%, 80%, 90%, 100%, 150%, 200%, 250%, 300%, 400%, 500%, 600%, 700%, 800%, or 900% higher than the solubility of an amorphous or crystalline form of Compound 1. In some embodiments, solubility is measured as grams of Compound 1 thiamine HCl co-crystal soluble in 100 g water.

### 4. Compound 1 Ferric Chloride Co-Crystal

In one aspect, provided herein is a co-crystal of Compound 1 and ferric chloride.

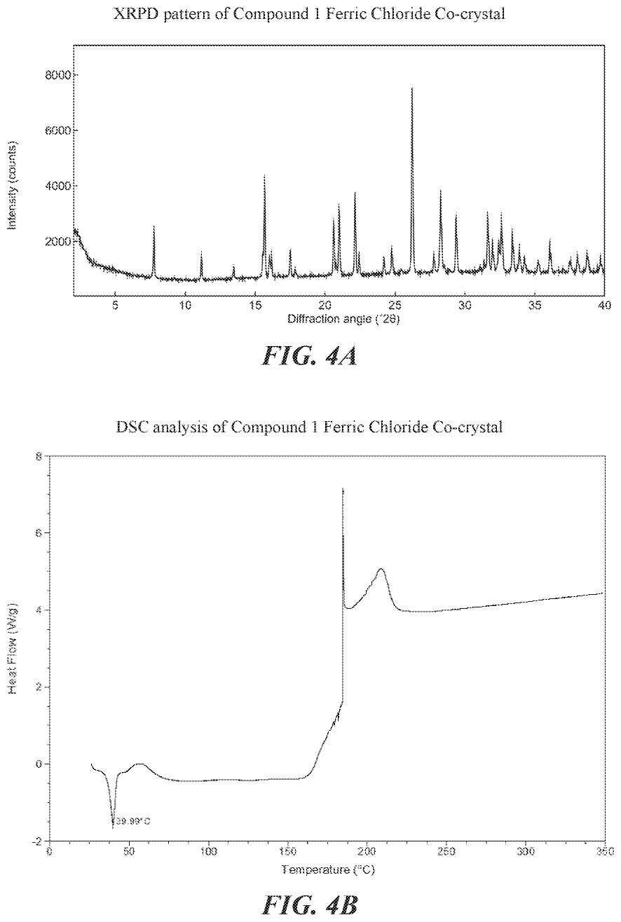

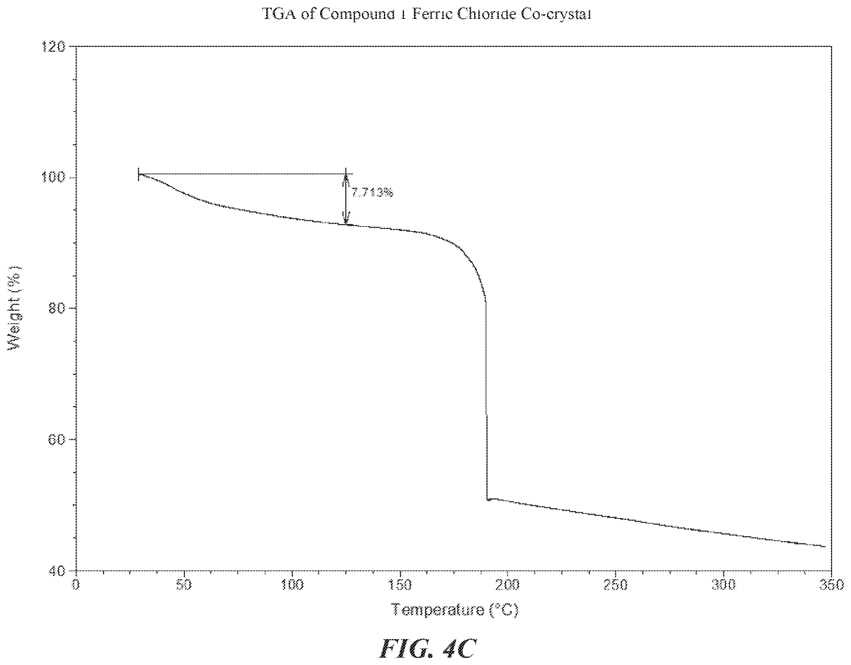

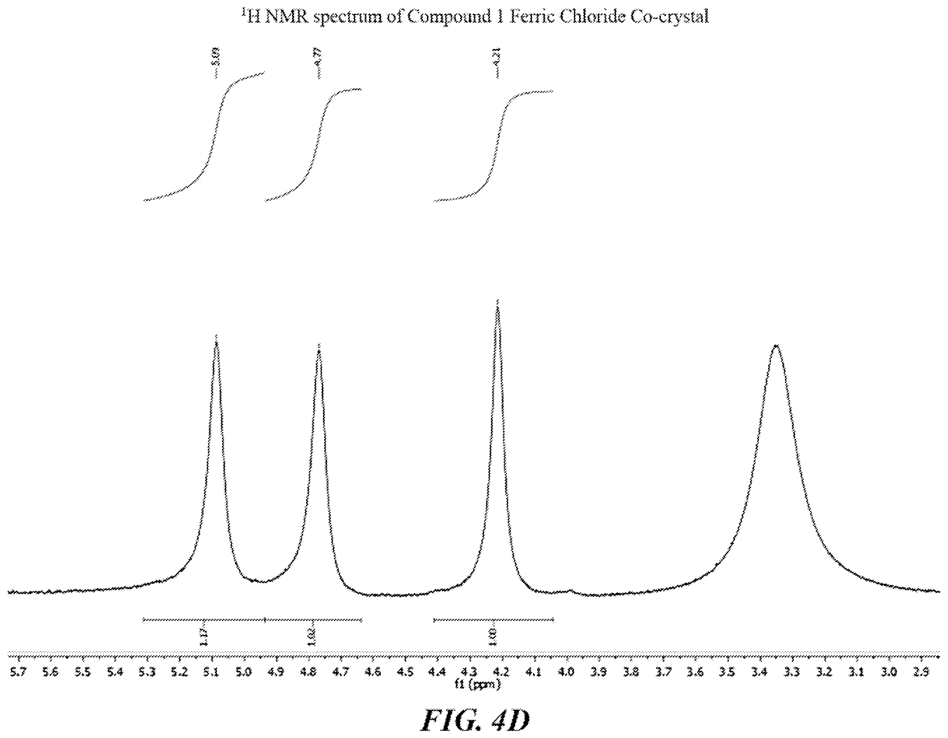

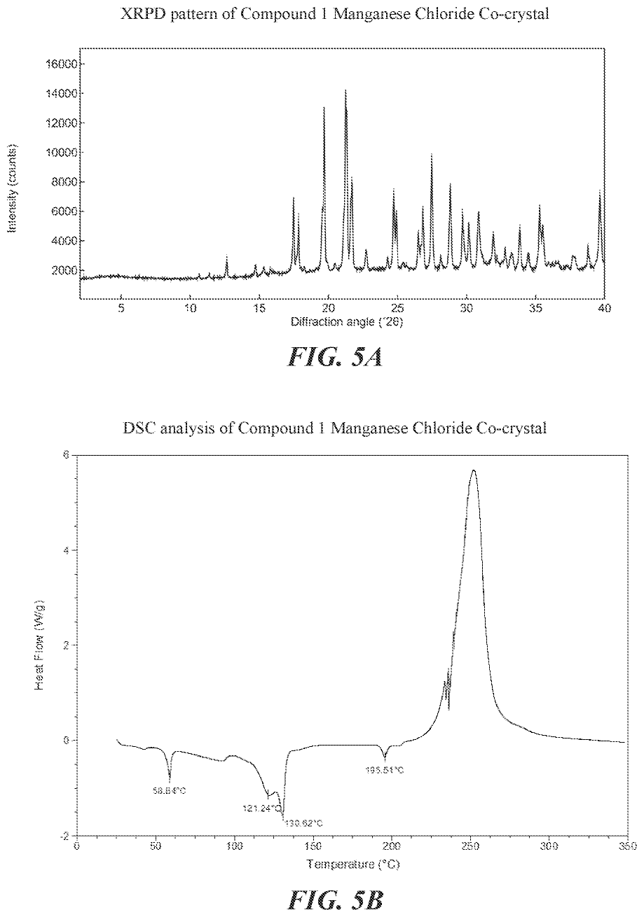

In some embodiments, the Compound 1 ferric chloride co-crystal has an XRPD pattern displaying at least one, at least two, at least three, at least four, at least five, at least six, at least seven, at least eight, at least nine, at least ten or more of the peaks at angles 2-theta in the XRPD pattern substantially as shown in FIG. 4A or as provided in Table 4B. In some embodiments, the Compound 1 ferric chloride co-crystal has an XRPD pattern displaying at least one, at least two, at least three, at least four, at least five, at least six, at least seven, at least eight, at least nine, at least ten or more of the peaks at angles 2-theta with the greatest intensity in the XRPD pattern substantially as shown in FIG. 4A or as provided in Table 4B. It should be understood that relative intensities can vary depending on a number of factors, including sample preparation, mounting, and the instrument and analytical procedure and settings used to obtain the spectrum. Relative peak intensities and peak assignments can vary within experimental error. In some embodiments, peak assignments listed herein, including for the Compound 1 ferric chloride co-crystal, can vary by about ±2.0 degrees. ±1.5 degrees, ±1.0 degrees, ±0.9 degrees, ±0.8 degrees, ±0.7 degrees, ±0.6 degrees, ±0.5 degrees, ±0.4 degrees, ±0.3 degrees, ±0.2 degrees, or ±0.1 degrees 2-theta.

In some embodiments, the Compound 1 ferric chloride co-crystal has an XRPD pattern comprising one or more peaks at angles 2-theta of about 7.8±δx, 13.5±δx, and 15.7±δx degrees. In some embodiments, the Compound 1 ferric chloride co-crystal has an XRPD pattern comprising peaks at angles 2-theta of about 7.8±δx, 11.2±δx, 13.5±δx, 15.7±δx, 16.0±δx and 16.2±δx degrees. In some embodiments, the Compound 1 ferric chloride co-crystal has an XRPD pattern comprising peaks at angles 2-theta of about 7.8±δx, 11.2±δx, 13.5±δx, 15.7±δx, 16.0±δx, 16.2±δx, 17.5±δx, 17.9±δx, 20.6±δx, 21.0±δx, 22.1±δx, 22.4±δx, 24.2±δx, 24.8±δx, 26.2±δx, 27.8±δx, 28.3±δx, 29.4±δx, 31.1±δx, 31.4±δx, 31.6±δx, 32.0±δx, 32.4±δx, 32.6±δx, 33.4±δx, 33.9±δx, 34.2±δx, 35.2±δx, 36.1±δx, 37.6±δx, 38.0±δx, 38.7±δx, or 39.7±δx degrees, or any combination thereof. In some embodiments, δx is 0 degrees 2-theta. In some embodiments, δx is 0.2 degrees 2-theta. In some embodiments, δx is 0.1, 0.2, 0.3, 0.4, 0.5, 0.6, 0.7, 0.8, 0.9, 1.0, 1.5, 2.0, 2.5, 3.0, 4.0, 5.0, 6.0, 7.0, 8.0, 9.0, or 10 degrees 2-theta.

In some embodiments, the Compound 1 ferric chloride co-crystal has a differential scanning calorimetry (DSC) graph substantially as shown in FIG. 4B. In some embodiments, the Compound 1 ferric chloride co-crystal is characterized by having a DSC graph comprising an endothermic peak at about 40° C. In some embodiments, the Compound 1 ferric chloride co-crystal is characterized by having a DSC graph comprising an endothermic peak at 40° C.±δ_{DSC}, where δ_{DSC} is 0.1, 0.2, 0.3, 0.4, 0.5, 0.6, 0.7, 0.8, 0.9, 1, 1.25, 1.5, 1.75, 2, 2.5, 3, 3.5, 4, 4.5, 5, 6, 7, 8, 9, or 10° C. In some embodiments. δ_{DSC} is 2° C. In some embodiments, the Compound 1 ferric chloride co-crystal is characterized by having a DSC graph comprising an endothermic peak between about 38° C. and about 42° C.

In some embodiments, the Compound 1 ferric chloride co-crystal melts at or has a melting point at about 40° C. as determined by DSC. In some embodiments, the Compound 1 ferric chloride co-crystal melts at or has a melting point at about 40° C.±δ_{DSC} as determined by DSC, where δ_{DSC} is 0.1, 0.2, 0.3, 0.4, 0.5, 0.6, 0.7, 0.8, 0.9, 1, 1.25, 1.5, 1.75, 2, 2.5, 3, 3.5, 4, 4.5, 5, 6, 7, 8, 9, or 10° C. In some embodiments, δ_{DSC} is 2° C. In some embodiments, the Compound 1 ferric chloride co-crystal melts between about 38° C. and about 42° C. as determined by DSC.

In some embodiments, Compound 1 ferric chloride co-crystal has a thermogravimetric analysis (TGA) graph substantially as shown in FIG. 4C. In some embodiments, Compound 1 ferric chloride co-crystal is a dihydrate as determined by TGA. In some embodiments, % weight decrease of Compound 1 ferric chloride co-crystal as determined by TGA from about 25° C. to about 125° C. is about 7.7%. In some embodiments, % weight decrease of Compound 1 ferric chloride co-crystal as determined by TGA from about 25° C. to about 125° C. is about 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 10.5%, 11%, 11.5%, 12%, 12.5%, 13%, 13.5%, 14%, 14.5%, 15%, 16%, 17%, 18%, 19%, or 20%. In some embodiments, % weight decrease of Compound 1 ferric chloride co-crystal as determined by TGA from about 25° C. to about 125° C. is at least about 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 10.5%, 11%, 11.5%, 12%, 12.5%, 13%, 13.5%, 14%, 14.5%, 15%, 16%, 17%, 18%, 19%, or 20%.

In some embodiments, Compound 1 ferric chloride co-crystal has a ¹H NMR spectrum substantially as shown in FIG. 4D. In some embodiments, the Compound 1 ferric chloride co-crystal has ¹H NMR spectrum comprising one or more peaks at about 4.21, 4.77, or 5.09 ppm, or any combination thereof. In some embodiments, the Compound 1 ferric chloride co-crystal has an ¹H NMR spectrum comprising one or more peaks at 4.21±δ_{N}, 4.77±δ_{N}, or 5.09±δ_{N} ppm, or any combination thereof. In some embodiments, δ_{N} is 0.01, 0.05, 0.1, 0.2, 0.3, 0.4, 0.5, 0.6, 0.7, 0.8, 0.9, 0.10, 0.15, 0.2, 0.25, 0.3, 0.35, 0.4, 0.45, 0.5, 0.75, 1, 1.25, 1.5, 2, 3, 4, or 5 ppm. In some embodiments, δ_{N} is 0.2 ppm.

In some embodiments, Compound 1 ferric chloride co-crystal is crystalline. In some embodiments, Compound 1 ferric chloride co-crystal is unsolvated. In some embodiments. Compound 1 ferric chloride co-crystal is dihydrate. In some embodiments, Compound 1 ferric chloride co-crystal is not impact-sensitive. In some embodiments, Compound 1 ferric chloride co-crystal is less impact-sensitive than an Compound 1 crystal.

In some embodiments, the solubility of Compound 1 ferric chloride co-crystal in water or in a mixture of water with other solvents is higher than the solubility of an amorphous or crystalline form of Compound 1. In some embodiments, the solubility of Compound 1 ferric chloride co-crystal herein may be significantly higher than the solubility of an amorphous or crystalline form of Compound 1. In some embodiments, the solubility of Compound 1 ferric chloride co-crystal is at least about 1%, 5%, 10%, 15%, 20%, 30%, 40%, 50%, 60%, 70%, 80%, 90%, 100%, 150%, 200%, 250%, 300%, 400%, 500%, 600%, 700%, 800%, or 900% higher than the solubility of an amorphous or crystalline form of Compound 1. In some embodiments, solubility is measured as grams of Compound 1 ferric chloride co-crystal soluble in 100 g water.

### 5. Compound 1 Manganese Chloride Co-Crystal

In one aspect, provided herein is a co-crystal of Compound 1 and manganese chloride.

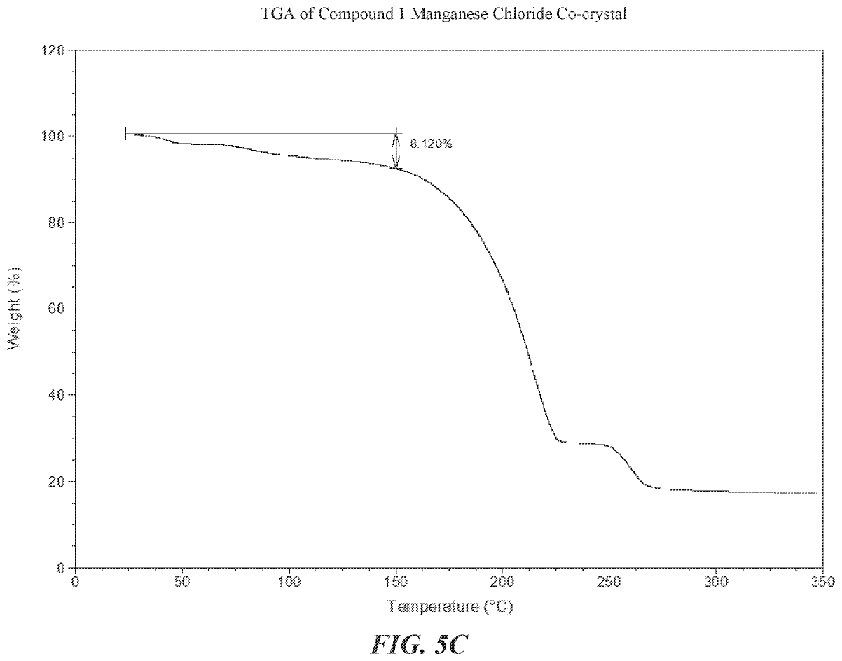

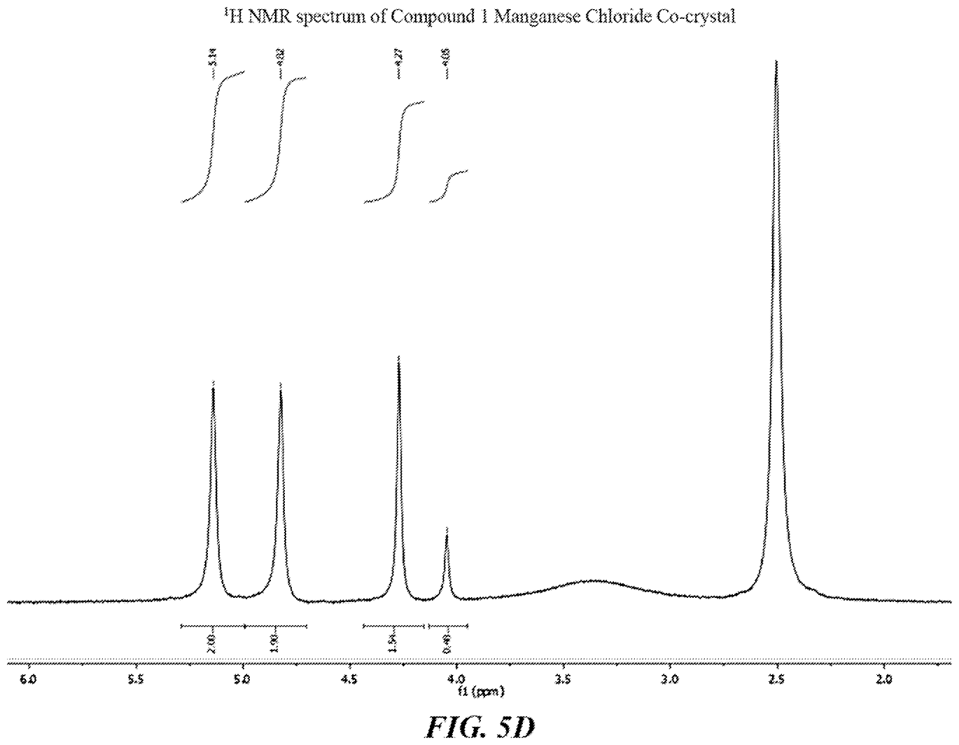

In some embodiments, the Compound 1 manganese chloride co-crystal has an XRPD pattern displaying at least one, at least two, at least three, at least four, at least five, at least six, at least seven, at least eight, at least nine, at least ten or more of the peaks at angles 2-theta in the XRPD pattern substantially as shown in FIG. 5A or as provided in Table 5B. In some embodiments, the Compound 1 manganese chloride co-crystal has an XRPD pattern displaying at least one, at least two, at least three, at least four, at least five, at least six, at least seven, at least eight, at least nine, at least ten or more of the peaks at angles 2-theta with the greatest intensity in the XRPD pattern substantially as shown in FIG. 5A or as provided in Table 5B. It should be understood that relative intensities can vary depending on a number of factors, including sample preparation, mounting, and the instrument and analytical procedure and settings used to obtain the spectrum. Relative peak intensities and peak assignments can vary within experimental error. In some embodiments, peak assignments listed herein, including for the Compound 1 manganese chloride co-crystal, can vary by about ±2.0 degrees, ±1.5 degrees, ±1.0 degrees, ±0.9 degrees, ±0.8 degrees, ±0.7 degrees, ±0.6 degrees, ±0.5 degrees, ±0.4 degrees, ±0.3 degrees, ±0.2 degrees, or ±0.1 degrees 2-theta.

In some embodiments, the Compound 1 manganese chloride co-crystal has an XRPD pattern comprising one or more peaks at angles 2-theta of about 12.7±δx, 14.7±δx, and 15.3±δx degrees. In some embodiments, the Compound 1 manganese chloride co-crystal has an XRPD pattern comprising peaks at angles 2-theta of about 10.6±δx, 11.4±δx, 12.7±δx, 14.7±δx, 15.3±δx and 15.8±δx degrees. In some embodiments, the Compound 1 manganese chloride co-crystal has an XRPD pattern comprising peaks at angles 2-theta of about 10.6±δx, 11.4±δx, 12.7±δx, 14.7±δx, 15.3±δx, 15.8±δx, 17.5±δx, 17.8±δx, 18.3±δx, 19.7±δx, 20.5±δx, 21.2±δx, 21.7±δx, 22.7±δx, 24.3±δx, 24.7±δx, 24.9±δx, 25.4±δx, 26.5±δx, 26.8±δx, 27.5±δx, 28.1±δx, 28.8±δx, 29.7±δx, 30.1±δx, 30.9±δx, 31.9±δx, 32.2±δx, 32.8±δx, 33.2±δx, 33.8±δx, 34.4±δx, 35.3±δx, 35.5±δx, 37.7±δx, 38.8±δx, or 39.6±δx degrees, or any combination thereof. In some embodiments, δx is 0 degrees 2-theta. In some embodiments, δx is 0.2 degrees 2-theta. In some embodiments, δx is 0.1, 0.2, 0.3, 0.4, 0.5, 0.6, 0.7, 0.8, 0.9, 1.0, 1.5, 2.0, 2.5, 3.0, 4.0, 5.0, 6.0, 7.0, 8.0, 9.0, or 10 degrees 2-theta.

In some embodiments, the Compound 1 manganese chloride co-crystal has a differential scanning calorimetry (DSC) graph substantially as shown in FIG. 5B. In some embodiments, the Compound 1 manganese chloride co-crystal is characterized as having multiple endotherm events below 200° C. as determined by DSC. In some embodiments, the Compound 1 manganese chloride co-crystal is characterized by having a DSC graph comprising one or more endothermic peaks at about 58.8° C., 121.2° C., 130.6° C., or 195.5° C. In some embodiments, the Compound 1 manganese chloride co-crystal is characterized by having a DSC graph comprising one or more endothermic peaks at about 58.8° C.±γ_{DSC}, 121.2° C.±γ_{DSC}, 130.6° C.±γ_{DSC}, or 195.5° C.±γ_{DSC}, where δ_{DSC} is 0.1, 0.2, 0.3, 0.4, 0.5, 0.6, 0.7, 0.8, 0.9, 1, 1.25, 1.5, 1.75, 2, 2.5, 3, 3.5, 4, 4.5, 5, 6, 7, 8, 9, or 10° C. In some embodiments, γ_{DSC} is 2° C. In some embodiments, the Compound 1 manganese chloride co-crystal is characterized by having a DSC graph comprising one or more endothermic peaks between about 57° C. and about 61° C., between about 119° C. and about 223° C., between about 129° C. and about 133° C., or between about 194° C. and about 198° C.

In some embodiments, the Compound 1 manganese chloride co-crystal melts at or has a melting point at about 58.8° C., 121.2° C., 130.6° C., or 195.5° C. as determined by DSC. In some embodiments, the Compound 1 manganese chloride co-crystal melts at or has a melting point at about 58.8° C.±γ_{DSC}, 121.2° C.±δ_{DSC}, 130.6° C.±δ_{DSC}, or 195.5° C.±δ_{DSC} as determined by DSC, where δ_{DSC} is 0.1, 0.2, 0.3, 0.4, 0.5, 0.6, 0.7, 0.8, 0.9, 1, 1.25, 1.5, 1.75, 2, 2.5, 3, 3.5, 4, 4.5, 5, 6, 7, 8, 9, or 10° C. In some embodiments. δ_{DSC} is 2° C. In some embodiments, the Compound 1 manganese chloride co-crystal melts between about 57° C. and about 61° C., between about 119° C. and about 223° C., between about 129° C. and about 133° C., or between about 194° C. and about 198° C. as determined by DSC.

In some embodiments, Compound 1 manganese chloride co-crystal has a thermogravimetric analysis (TGA) graph substantially as shown in FIG. 5C. In some embodiments, Compound 1 manganese chloride co-crystal is a dihydrate as determined by TGA. In some embodiments. % weight decrease of Compound 1 manganese chloride co-crystal as determined by TGA from about 25° C. to about 150° C. is about 8.1%. In some embodiments, % weight decrease of Compound 1 manganese chloride co-crystal as determined by TGA from about 25° C. to about 150° C. is about 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 10.5%, 11%, 11.5%, 12%, 12.5%, 13%, 13.5%, 14%, 14.5%, 15%, 16%, 17%, 18%, 19%, or 20%. In some embodiments, % weight decrease of Compound 1 manganese chloride co-crystal as determined by TGA from about 25° C. to about 150° C. is at least about 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 10.5%, 11%, 11.5%, 12%, 12.5%, 13%, 13.5%, 14%, 14.5%, 15%, 16%, 17%, 18%, 19%, or 20%.

In some embodiments, Compound 1 manganese chloride co-crystal has a ¹H NMR spectrum substantially as shown in FIG. 5D. In some embodiments, the Compound 1 manganese chloride co-crystal has ¹H NMR spectrum comprising one or more peaks at about 4.05, 4.27, 4.82, or 5.14 ppm, or any combination thereof. In some embodiments, the Compound 1 manganese chloride co-crystal has an ¹H NMR spectrum comprising one or more peaks at 4.05δ_{N}, 4.27±δ_{N}, 4.82±δ_{N}, or 5.14±δ_{N} ppm, or any combination thereof. In some embodiments, δ_{N} is 0.01, 0.05, 0.1, 0.2, 0.3, 0.4, 0.5, 0.6, 0.7, 0.8, 0.9, 0.10, 0.15, 0.2, 0.25, 0.3, 0.35, 0.4, 0.45, 0.5, 0.75, 1, 1.25, 1.5, 2, 3, 4, or 5 ppm. In some embodiments, δ_{N} is 0.2 ppm.

In some embodiments, Compound 1 manganese chloride co-crystal is crystalline. In some embodiments. Compound 1 manganese chloride co-crystal is unsolvated. In some embodiments, Compound 1 manganese chloride co-crystal is dihydrate. In some embodiments, Compound 1 manganese chloride co-crystal is not impact-sensitive.

In some embodiments, the solubility of Compound 1 manganese chloride co-crystal in water or in a mixture of water with other solvents is higher than the solubility of an amorphous or crystalline form of Compound 1. In some embodiments, the solubility of Compound 1 manganese chloride co-crystal herein may be significantly higher than the solubility of an amorphous or crystalline form of Compound 1. In some embodiments, the solubility of Compound 1 manganese chloride co-crystal is at least about 1%, 5%, 10%, 15%, 20%, 30%, 40%, 50%, 60%, 70%, 80%, 90%, 100%, 150%, 200%, 250%, 300%, 400%, 500%, 600%, 700%, 800%, or 900% higher than the solubility of an amorphous or crystalline form of Compound 1. In some embodiments, solubility is measured as grams of Compound 1 manganese chloride co-crystal soluble in 100 g water.

### 6. Compound 1 Zinc Chloride Co-Crystal

In one aspect, provided herein is a co-crystal of Compound 1 and zinc chloride.

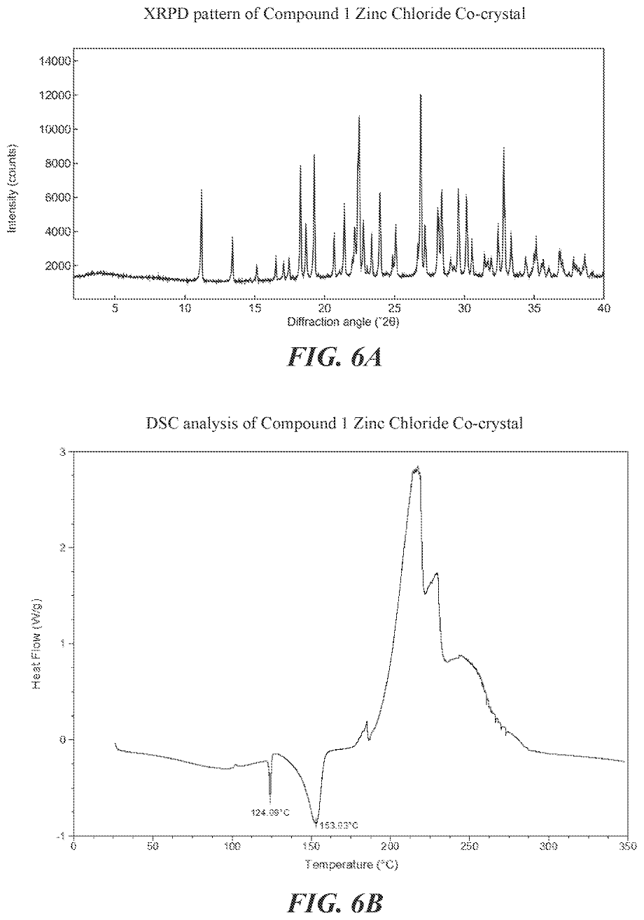

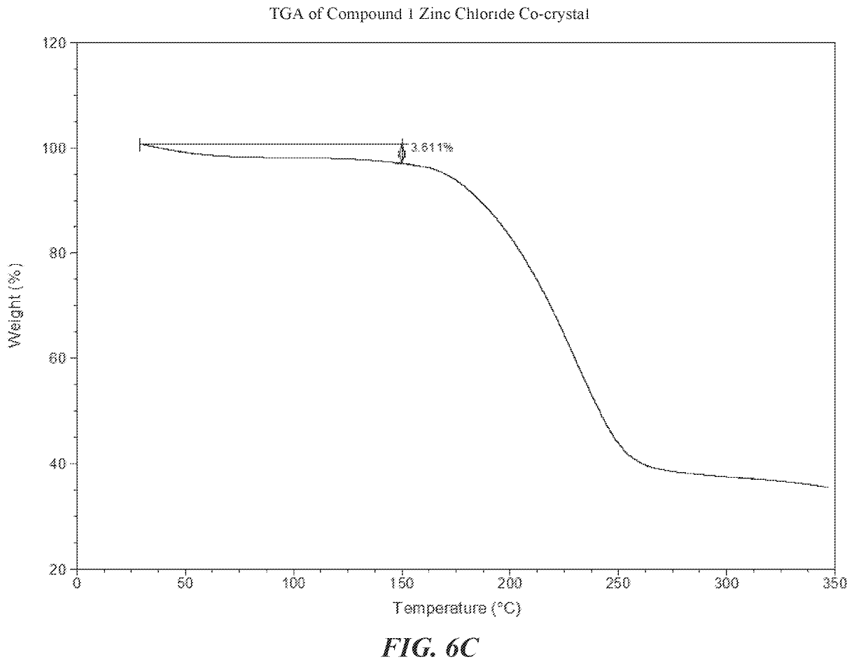

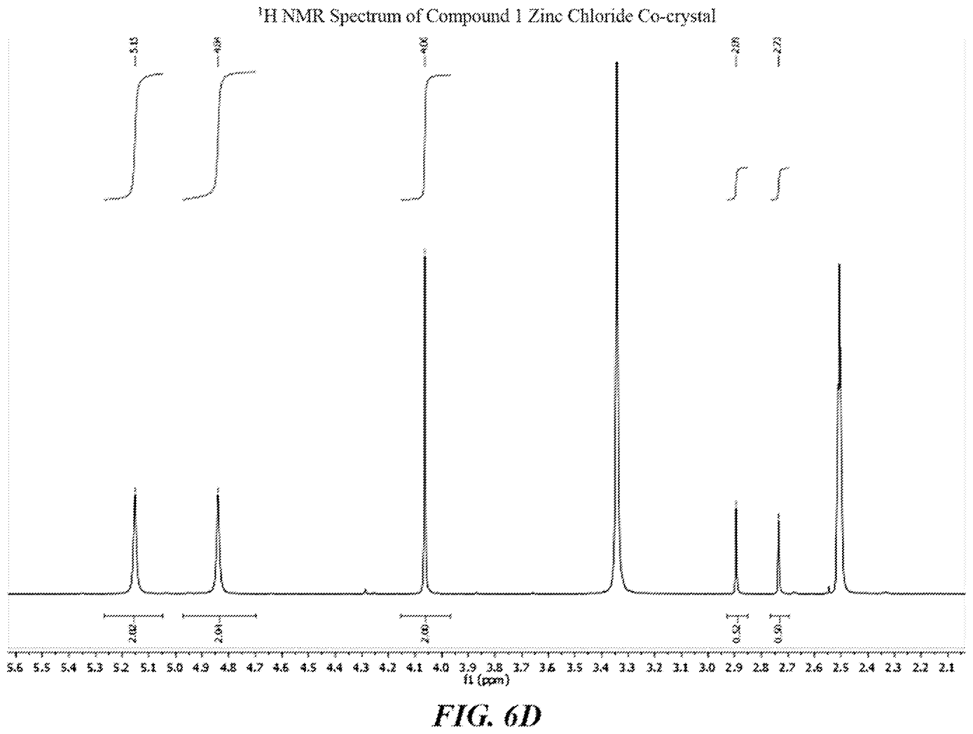

In some embodiments, the Compound 1 zinc chloride co-crystal has an XRPD pattern displaying at least one, at least two, at least three, at least four, at least five, at least six, at least seven, at least eight, at least nine, at least ten or more of the peaks at angles 2-theta in the XRPD pattern substantially as shown in FIG. 6A or as provided in Table 6B. In some embodiments, the Compound 1 zinc chloride co-crystal has an XRPD pattern displaying at least one, at least two, at least three, at least four, at least five, at least six, at least seven, at least eight, at least nine, at least ten or more of the peaks at angles 2-theta with the greatest intensity in the XRPD pattern substantially as shown in FIG. 6A or as provided in Table 6B. It should be understood that relative intensities can vary depending on a number of factors, including sample preparation, mounting, and the instrument and analytical procedure and settings used to obtain the spectrum. Relative peak intensities and peak assignments can vary within experimental error. In some embodiments, peak assignments listed herein, including for the Compound 1 zinc chloride co-crystal, can vary by about ±2.0 degrees, ±1.5 degrees, ±1.0 degrees, ±0.9 degrees, ±0.8 degrees, ±0.7 degrees, ±0.6 degrees, ±0.5 degrees, ±0.4 degrees, ±0.3 degrees, ±0.2 degrees, or ±0.1 degrees 2-theta.

In some embodiments, the Compound 1 zinc chloride co-crystal has an XRPD pattern comprising one or more peaks at angles 2-theta of about 13.4±δx, 15.1±δx, and 17.1±δx degrees. In some embodiments, the Compound 1 zinc chloride co-crystal has an XRPD pattern comprising peaks at angles 2-theta of about 11.2±δx, 13.4±δx, 15.1±δx, 16.5±δx and 17.1±δx degrees. In some embodiments, the Compound 1 zinc chloride co-crystal has an XRPD pattern comprising peaks at angles 2-theta of about 11.2±δx, 13.4±δx, 15.1±δx, 16.5±δx, 17.1±δx, 17.5±δx, 18.3±δx, 18.7±δx, 19.3±δx, 20.7±δx, 21.4±δx, 22.0±δx, 22.1±δx, 22.5±δx, 22.8±δx, 23.0±δx, 23.4±δx, 24.0±δx, 24.9±δx, 25.1±δx, 26.9±δx, 27.2±δx, 28.1±δx, 28.4±δx, 29.0±δx, 29.3±δx, 29.6±δx, 30.1±δx, 30.5±δx, 31.4±δx, 31.7±δx, 31.9±δx, 32.4±δx, 32.8±δx, 33.3±δx, 34.4±δx, 35.1±δx, 35.6±δx, 36.0±δx, 36.8±δx, 37.0±δx, 37.8±δx, or 38.6±δx degrees, or any combination thereof. In some embodiments, δx is 0 degrees 2-theta. In some embodiments, δx is 0.2 degrees 2-theta. In some embodiments, δx is 0.1, 0.2, 0.3, 0.4, 0.5, 0.6, 0.7, 0.8, 0.9, 1.0, 1.5, 2.0, 2.5, 3.0, 4.0, 5.0, 6.0, 7.0, 8.0, 9.0, or 10 degrees 2-theta.

In some embodiments, the Compound 1 zinc chloride co-crystal has a differential scanning calorimetry (DSC) graph substantially as shown in FIG. 6B. In some embodiments, the Compound 1 zinc chloride co-crystal is characterized by having a DSC graph comprising one or more endothermic peaks at about 124.1° C. or 153° C. In some embodiments, the Compound 1 zinc chloride co-crystal is characterized by having a DSC graph comprising one or more endothermic peaks at about 124.1° C.±δ_{DSC} or 153° C.±γ_{DSC}, where δ_{DSC} is 0.1, 0.2, 0.3, 0.4, 0.5, 0.6, 0.7, 0.8, 0.9, 1, 1.25, 1.5, 1.75, 2, 2.5, 3, 3.5, 4, 4.5, 5, 6, 7, 8, 9, or 10° C. In some embodiments, δ_{DSC} is 2° C. In some embodiments, the Compound 1 zinc chloride co-crystal is characterized by having a DSC graph comprising one or more endothermic peaks between about 122° C. and about 126° C. or between about 151° C. and about 155° C.

In some embodiments, the Compound 1 zinc chloride co-crystal melts at or has a melting point at about 124.1° C. or 153° C. as determined by DSC. In some embodiments, the Compound 1 zinc chloride co-crystal melts at or has a melting point at about 124.1° C.±γ_{DSC} or 153° C.±γ_{DSC} as determined by DSC, where γ_{DSC} is 0.1, 0.2, 0.3, 0.4, 0.5, 0.6, 0.7, 0.8, 0.9, 1, 1.25, 1.5, 1.75, 2, 2.5, 3, 3.5, 4, 4.5, 5, 6, 7, 8, 9, or 10° C. In some embodiments, γ_{DSC} is 2° C. In some embodiments, the Compound 1 zinc chloride co-crystal melts between about 122° C. and about 126° C. or between about 151° C. and about 155° C. as determined by DSC.

In some embodiments, Compound 1 zinc chloride co-crystal has a thermogravimetric analysis (TGA) graph substantially as shown in FIG. 6C. In some embodiments, Compound 1 zinc chloride co-crystal is a hydrate as determined by TGA. In some embodiments, % weight decrease of Compound 1 zinc chloride co-crystal as determined by TGA from about 25° C. to about 150° C. is about 3.6%. In some embodiments, % weight decrease of Compound 1 zinc chloride co-crystal as determined by TGA from about 25° C. to about 150° C. is about 0.1%, 0.2%, 0.3%, 0.4%, 0.5%, 1%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.7%, 2.8%, 2.9%, 3%, 3.1%, 3.2%, 3.3%, 3.4%, 3.5%, 3.6%, 3.7%, 3.8%, 3.9%, 4%, 4.5%, 5%, 5.5%, 6%, 6.5%, 7%, 7.5%, 8%, 8.5%, 9%, 9.5%, or 10%. In some embodiments, % weight decrease of Compound 1 zinc chloride co-crystal as determined by TGA from about 25° C. to about 150° C. is at least about 0.1%, 0.2%, 0.3%, 0.4%, 0.5%, 1%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.7%, 2.8%, 2.9%, 3%, 3.1%, 3.2%, 3.3%, 3.4%, 3.5%, 3.6%, 3.7%, 3.8%, 3.9%, 4%, 4.5%, 5%, 5.5%, 6%, 6.5%, 7%, 7.5%, 8%, 8.5%, 9%, 9.5%, or 10%.

In some embodiments, Compound 1 zinc chloride co-crystal has a ¹H NMR spectrum substantially as shown in FIG. 6D, where peaks at 2.7 and 2.9 ppm are observed impurities. In some embodiments, the Compound 1 zinc chloride co-crystal has ¹H NMR spectrum comprising one or more peaks at about 4.06, 4.84, or 5.15 ppm, or any combination thereof. In some embodiments, the Compound 1 zinc chloride co-crystal has an ¹H NMR spectrum comprising one or more peaks at 4.06±δ_{N}, 4.84±δ_{N}, or 5.15±δ_{N} ppm, or any combination thereof. In some embodiments, δ_{N} is 0.01, 0.05, 0.1, 0.2, 0.3, 0.4, 0.5, 0.6, 0.7, 0.8, 0.9, 0.10, 0.15, 0.2, 0.25, 0.3, 0.35, 0.4, 0.45, 0.5, 0.75, 1, 1.25, 1.5, 2, 3, 4, or 5 ppm. In some embodiments, δ_{N} is 0.2 ppm.

In some embodiments, Compound 1 zinc chloride co-crystal is crystalline. In some embodiments, Compound 1 zinc chloride co-crystal is unsolvated. In some embodiments, Compound 1 zinc chloride co-crystal is hydrate. In some embodiments, Compound 1 zinc chloride co-crystal is not impact-sensitive. In some embodiments, Compound 1 zinc chloride co-crystal is less impact-sensitive than an Compound 1 crystal.

In some embodiments, the solubility of Compound 1 zinc chloride co-crystal in water or in a mixture of water with other solvents is higher than the solubility of an amorphous or crystalline form of Compound 1. In some embodiments, the solubility of Compound 1 zinc chloride co-crystal herein may be significantly higher than the solubility of an amorphous or crystalline form of Compound 1. In some embodiments, the solubility of Compound 1 zinc chloride co-crystal is at least about 1%, 5%, 10%, 15%, 20%, 30%, 40%, 50%, 60%, 70%, 80%, 90%, 100%, 150%, 200%, 250%, 300%, 400%, 500%, 600%, 700%, 800%, or 900% higher than the solubility of an amorphous or crystalline form of Compound 1. In some embodiments, solubility is measured as grams of Compound 1 zinc chloride co-crystal soluble in 100 g water.

### II. Methods of Preparation

Compound 1 can be synthesized by synthetic methods known to the skilled artisan, for example, as described in U.S. Pat. No. 7,507,842. Further suitable methods for the co-crystals described herein are described in, e.g., U.S. Pat. Nos. 10,342,778 or 9,987,270.

In some aspect, provided is a method of preparing Compound 1 calcium chloride co-crystal. In some embodiments, the method comprises combining or mixing Compound 1 and calcium chloride. In some embodiments, the method comprises combining or mixing molar equivalents of Compound 1 and calcium chloride. In some embodiments, the method comprises mixing a solvent or a solvent mixture, e.g., alcohol, water, or a combination thereof, or a mixture of ethanol and water, with Compound 1 and calcium chloride. In some embodiments, mixing or the mixing step is performed at room temperature. In some embodiments, ethanol and water are provided in about 95:5 volume ratio. In some embodiments, ethanol and water are provided in about 99:1, 95:5, 90:10, 85:15, 80:20, 70:30, 60:40, or 50:50 volume ratio. In some embodiments, the method comprises mixing the solvent or the solvent mixture with Compound 1 and calcium chloride to form a slurry. In some embodiments, the method comprises stirring the slurry. In some embodiments, stirring or the stirring step is performed at room temperature. In some embodiments, the method comprises filtering the slurry to obtain the solid content of the slurry. In some embodiments, the method comprises drying the collected solid content.

In some aspect, provided is a method of preparing Compound 1 pyridoxine HCl co-crystal. In some embodiments, the method comprises combining or mixing Compound 1 and pyridoxine HCl. In some embodiments, the method comprises combining or mixing molar equivalents of Compound 1 and pyridoxine HCl. In some embodiments, the method comprises mixing a solvent or a solvent mixture, e.g., alcohol, water, or a combination thereof, or a mixture of ethanol and water, with Compound 1 and pyridoxine HCl. In some embodiments, mixing or the mixing step is performed at room temperature. In some embodiments, ethanol and water are provided in about 95:5 volume ratio. In some embodiments, ethanol and water are provided in about 99:1, 95:5, 90:10, 85:15, 80:20, 70:30, 60:40, or 50:50 volume ratio. In some embodiments, the method comprises mixing the solvent or the solvent mixture with Compound 1 and pyridoxine HCl to form a slurry. In some embodiments, the method comprises stirring the slurry. In some embodiments, stirring is performed at room temperature. In some embodiments, the method comprises filtering the slurry to obtain the solid content of the slurry. In some embodiments, the method comprises drying the collected solid content.

In some aspect, provided is a method of preparing Compound 1 thiamine HCl co-crystal. In some embodiments, the method comprises combining or mixing Compound 1 and thiamine HCl. In some embodiments, the method comprises combining or mixing molar equivalents of Compound 1 and thiamine HCl. In some embodiments, the method comprises mixing a solvent or a solvent mixture, e.g., alcohol, water, or a combination thereof, or a mixture of ethanol and water, with Compound 1 and thiamine HCl. In some embodiments, mixing or the mixing step is performed at room temperature. In some embodiments, ethanol and water are provided in about 95:5 volume ratio. In some embodiments, ethanol and water are provided in about 99:1, 95:5, 90:10, 85:15, 80:20, 70:30, 60:40, or 50:50 volume ratio. In some embodiments, the method comprises mixing the solvent or the solvent mixture with Compound 1 and thiamine HCl to form a slurry. In some embodiments, the method comprises stirring the slurry at room temperature. In some embodiments, stirring or the stirring step is performed at room temperature. In some embodiments, the method comprises filtering the slurry to obtain the solid content of the slurry. In some embodiments, the method comprises drying the collected solid content.

In some aspect, provided is a method of preparing Compound 1 ferric chloride co-crystal. In some embodiments, the method comprises combining or mixing Compound 1 and ferric chloride. In some embodiments, the method comprises combining or mixing molar equivalents of Compound 1 and ferric chloride. In some embodiments, the method comprises mixing a solvent or a solvent mixture, e.g., alcohol, water, or a combination thereof, a mixture of ethanol and water, with Compound 1 and ferric chloride. In some embodiments, mixing or the mixing step is performed at room temperature. In some embodiments, ethanol and water are provided in about 95:5 volume ratio. In some embodiments, ethanol and water are provided in about 99:1, 95:5, 90:10, 85:15, 80:20, 70:30, 60:40, or 50:50 volume ratio. In some embodiments, the method comprises mixing the solvent or the solvent mixture with Compound 1 and ferric chloride to form a slurry. In some embodiments, the method comprises stirring the slurry. In some embodiments, stirring or the stirring step is performed at room temperature. In some embodiments, the method comprises filtering the slurry to obtain the solid content of the slurry. In some embodiments, the method comprises drying the collected solid content.

In some aspect, provided is a method of preparing Compound 1 manganese chloride co-crystal. In some embodiments, the method comprises combining or mixing Compound 1 and manganese chloride. In some embodiments, the method comprises combining or mixing molar equivalents of Compound 1 and manganese chloride. In some embodiments, the method comprises mixing a solvent or a solvent mixture, e.g., alcohol, water, or a combination thereof, a mixture of ethanol and water, with Compound 1 and manganese chloride. In some embodiments, mixing or the mixing step is performed at room temperature. In some embodiments, ethanol and water are provided in about 95:5 volume ratio. In some embodiments, ethanol and water are provided in about 99:1, 95:5, 90:10, 85:15, 80:20, 70:30, 60:40, or 50:50 volume ratio. In some embodiments, the method comprises mixing the solvent or the solvent mixture with Compound 1 and manganese chloride to form a slurry. In some embodiments, the method comprises stirring the slurry. In some embodiments, stirring or the stirring step is performed at room temperature. In some embodiments, the method comprises filtering the slurry to obtain the solid content of the slurry. In some embodiments, the method comprises drying the collected solid content.

In some aspect, provided is a method of preparing Compound 1 zinc chloride co-crystal. In some embodiments, the method comprises combining or mixing Compound 1 and zinc chloride. In some embodiments, the method comprises combining or mixing molar equivalents of Compound 1 and zinc chloride. In some embodiments, the method comprises mixing a solvent or a solvent mixture, e.g., alcohol, water, or a combination thereof, a mixture of ethanol and water, with Compound 1 and zinc chloride. In some embodiments, mixing or the mixing step is performed at room temperature. In some embodiments, ethanol and water are provided in about 95:5 volume ratio. In some embodiments, ethanol and water are provided in about 99:1, 95:5, 90:10, 85:15, 80:20, 70:30, 60:40, or 50:50 volume ratio. In some embodiments, the method comprises mixing the solvent or the solvent mixture with Compound 1 and zinc chloride to form a slurry. In some embodiments, the method comprises stirring the slurry. In some embodiments, stirring or the stirring step is performed at room temperature. In some embodiments, the method comprises filtering the slurry to obtain the solid content of the slurry. In some embodiments, the method comprises drying the collected solid content.

### III. Pharmaceutical Compositions

In one aspect, provided herein are pharmaceutical compositions comprising co-crystals described herein, such as Compound 1 calcium chloride co-crystal, Compound 1 pyridoxine HCl co-crystal, Compound 1 thiamine HCl co-crystal, Compound 1 ferric chloride co-crystal, Compound 1 manganese chloride co-crystal, or Compound 1 zinc chloride co-crystal, or a mixture thereof. In some embodiments, the composition comprises Compound 1 calcium chloride co-crystal. In some embodiments, the composition comprises Compound 1 pyridoxine HCl co-crystal. In some embodiments, the composition comprises Compound 1 thiamine HCl co-crystal. In some embodiments, the composition comprises Compound 1 ferric chloride co-crystal. In some embodiments, the composition comprises Compound 1 manganese chloride co-crystal. In some embodiments, the composition comprises Compound 1 zinc chloride co-crystal. In some embodiments, the composition further comprises one or more pharmaceutically acceptable excipients, carriers, and/or diluents. In some embodiments, the composition comprises co-crystals described herein in a therapeutically effective amount.

In some embodiments, the pharmaceutical composition contains at least one active agent and a pharmaceutically acceptable carrier. The pharmaceutical composition described herein may be specially formulated for administration in liquid form, including those adapted for the following: (1) oral administration, for example, drenches (aqueous or non-aqueous solutions or suspensions), tablets (e.g., those targeted for buccal, sublingual, and/or systemic absorption), boluses, powders, granules, pastes for application to the tongue); and (2) parenteral administration by, for example, subcutaneous, intramuscular, intravenous or epidural injection as, for example, a sterile solution or suspension, or sustained-release formulation: (3) topical application, for example, as a cream, ointment, or a controlled-release patch or spray applied to the skin; (4) intravaginally or intrarectally, for example, as a pessary, cream or foam: (5) sublingually; (6) ocularly; (7) transdermally; or (8) nasally.

The amount of any of the co-crystals described herein which can be combined with a carrier material to produce a single dosage form will generally be that amount of the compound which produces a therapeutic effect. In some embodiments, out of one hundred percent, this amount will range from about 0.1 percent to about ninety-nine percent of co-crystal, from about 5 percent to about 70 percent, or from about 10 percent to about 30 percent.

In some embodiments, water is a preferred vehicle when the pharmaceutical compositions of the present invention is administered intravenously. In some embodiments, saline solutions and aqueous dextrose and glycerol solutions can also be employed as liquid vehicles, particularly for injectable solutions. In some embodiments, suitable pharmaceutical vehicles also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

In some embodiments, the present pharmaceutical compositions, if desired, can also contain minor amounts of wetting or emulsifying agents, or pH buffering agents. In addition, auxiliary, stabilizing, thickening, lubricating and coloring agents may be used.

In some embodiments, any of the pharmaceutical compositions described herein, may be manufactured by means of conventional mixing, dissolving, granulating, dragee-making, levigating, emulsifying, encapsulating, entrapping or lyophilizing processes. In some embodiments, pharmaceutical compositions may be formulated in conventional manner using one or more physiologically acceptable carriers, diluents, excipients or auxiliaries, which facilitate processing of compounds into preparations which can be used pharmaceutically. Proper formulation is dependent upon the route of administration chosen.

In some embodiments, the pharmaceutical compositions of the present invention are formulated in accordance with routine procedures as a pharmaceutical composition adapted for intravenous administration to human beings. In a preferred embodiment, the pharmaceutical compositions of the present invention are solutions in sterile isotonic aqueous buffer for intravenous administration. In some embodiments, for injection, any of the co-crystals described herein and/or pharmaceutical compositions thereof may be formulated in aqueous solutions, e.g., in physiologically compatible buffers such as Hanks solution, Ringer's solution, or physiological saline buffer. In some embodiments, the solution may contain formulatory agents such as suspending, stabilizing and/or dispersing agents. In some embodiments, when necessary, the pharmaceutical compositions may also include a solubilizing agent. In some embodiments, the pharmaceutical compositions for intravenous administration may optionally include a local anesthetic such as lignocaine to ease pain at the site of the injection. In some embodiments, the ingredients are supplied either separately or mixed together in unit dosage form, for example, as a lyophilized powder or water free concentrate in a hermetically sealed container such as an ampoule or sachette indicating the quantity of active agent. In some embodiments, when the pharmaceutical compositions of the present invention are administered by infusion, it can be dispensed, for example, with an infusion bottle containing sterile pharmaceutical grade water or saline. In other embodiments, when the pharmaceutical compositions of the present invention are administered by injection, an ampoule of sterile water for injection or saline can be provided so that the ingredients may be mixed prior to administration.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 0.1 percent to about ninety-nine percent of active ingredient, particularly from about 5 percent to about 70 percent, or from about 10 percent to about 30 percent.

In some embodiments, liquid dosage forms for oral administration of the compounds of the invention include pharmaceutically acceptable emulsions, microemulsions, solutions, aqueous or oily suspensions, syrups and elixirs. In some embodiments, in addition to the co-crystal, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

In some embodiments, besides inert diluents, the oral compositions can also include adjuvants such as wetting agents, emulsifying and suspending agents, sweetening, flavoring, coloring, perfuming and preservative agents. It may also be desirable to include isotonic agents, such as sugars, sodium chloride, and the like into the compositions. In other embodiments, prolonged absorption of the injectable pharmaceutical form may be brought about by the inclusion of agents which delay absorption such as aluminum monostearate and gelatin. Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

In some embodiments, suspensions, in addition to the co-crystals described herein, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

In some embodiments, pharmaceutical compositions of this invention suitable for parenteral administration comprise one or more compounds of the invention in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. In some embodiments, proper fluidity can be maintained, for example, by the use of coating materials, such as lecithin, by the maintenance of the required particle size in the case of dispersions, and by the use of surfactants.

In some embodiments, these compositions may also contain adjuvants such as preservatives, wetting agents, emulsifying agents and dispersing agents. In some embodiments, prevention of the action of microorganisms upon the subject compounds may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In other embodiments, prolonged absorption of the injectable pharmaceutical form may be brought about by the inclusion of agents which delay absorption such as aluminum monostearate and gelatin. In some embodiments, liquid drug formulations suitable for use with nebulizers and liquid spray devices and EHD aerosol devices typically include the composition with a pharmaceutically acceptable vehicle. In a preferred embodiment, the pharmaceutically acceptable vehicle is a liquid such as alcohol, water, polyethylene glycol or a perfluorocarbon. Optionally, another material may be added to alter the aerosol properties of the solution or suspension of compounds. In some embodiments, this material is liquid such as an alcohol, glycol, polyglycol or a fatty acid. Other methods of formulating liquid drug solutions or suspension suitable for use in aerosol devices are known to those of skill in the art (see, e.g., Biesalski, U.S. Pat. No. 5,112,598; Biesalski, U.S. Pat. No. 5,556,611).

In some embodiments, the preparations of the present invention may be given, for example, orally or parenterally. The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrasternal injection and infusion.

In some embodiments, systemic formulations include those designed for administration by injection, e.g., subcutaneous, intravenous, intramuscular, intrathecal or intraperitoneal injection, as well as those designed for transdermal, transmucosal, oral or pulmonary administration. Systemic formulations may be made in combination with a further active agent that improves mucociliary clearance of airway mucus or reduces mucous viscosity. These active agents include, but are not limited to, sodium channel blockers, antibiotics, N-acetyl cysteine, homocysteine and phospholipids.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

In some embodiments, the compositions described herein may be administered to humans and other animals for therapy by any suitable route of administration, including orally, nasally, as by, for example, a spray, rectally, intravaginally, parenterally, intracisternally and topically.

In some embodiments, for topical administration, the compositions described in the current application may be formulated as by powders, ointments or drops, including buccally and sublingually, solutions, gels, ointments, creams, suspensions, etc. as is well-known in the art.

In some embodiments, for buccal administration, the pharmaceutical compositions may take the form of tablets, lozenges, etc. formulated in conventional manner.

In some embodiments, for transmucosal administration, penetrants appropriate to the barrier to be permeated are used in the formulation. Such penetrants are generally known in the art.

In some embodiments, the present pharmaceutical compositions can take the form of solutions, suspensions, emulsion, tablets, pills, pellets, capsules, capsules containing liquids, powders, sustained-release formulations, suppositories, emulsions, aerosols, sprays, suspensions, or any other form suitable for use. In one embodiment, the pharmaceutically acceptable vehicle is a capsule (e.g., Grosswald et al., U.S. Pat. No. 5,698,155). A general discussion of the preparation of pharmaceutical compositions may be found in Remington, “The Science and Practice of Pharmacy,” 19th Edition.

In solid dosage forms of the invention for oral administration (capsules, tablets, pills, dragees, powders, granules, trouches and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid: (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds and surfactants, such as poloxamer and sodium lauryl sulfate; (7) wetting agents, such as, for example, cetyl alcohol, glycerol monostearate, and non-ionic surfactants; (8) absorbents, such as kaolin and bentonite clay; (9) lubricants, such as talc, calcium stearate, magnesium stearate, solid polyethylene glycols, sodium lauryl sulfate, zinc stearate, sodium stearate, stearic acid, and mixtures thereof; (10) coloring agents; and (11) controlled release agents such as crospovidone or ethyl cellulose. In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

In other embodiments, the present pharmaceutical compositions, when in capsule, tablet or pill form, may be coated to delay disintegration and absorption in the gastrointestinal tract, thereby providing a sustained action over an extended period of time. In some embodiments, selectively permeable membranes surrounding an osmotically active driving compound are also suitable for orally administered compounds. In some embodiments, in these later platforms, fluid from the environment surrounding the capsule is imbibed by the driving compound, which swells to displace the agent or agent composition through an aperture. In some embodiments, these delivery platforms can provide an essentially zero order delivery profile as opposed to the spiked profiles of immediate release formulations. In some embodiments, a time delay material such as glycerol monostearate or glycerol stearate may also be used. In some embodiments, oral compositions can include standard vehicles such as mannitol, lactose, starch, magnesium stearate, sodium saccharine, cellulose, magnesium carbonate, etc. In some embodiments, such vehicles are of pharmaceutical grade.

In some embodiments, any of the co-crystals described herein and/or pharmaceutical compositions thereof may also be formulated in rectal or vaginal pharmaceutical compositions such as suppositories or retention enemas, e.g., containing conventional suppository bases such as cocoa butter or other glycerides.

In some embodiments, the pharmaceutical composition may be configured as an ointment, paste, cream or gel. The ointment, paste, cream or gel may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

In other embodiments, the pharmaceutical composition may be configured as a transdermal patch. Transdermal patches have the added advantage of providing controlled delivery of a compound of the present invention to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the compound in a polymer matrix or gel. Ophthalmic formulations, eye ointments, powders, solutions and the like, are also contemplated as being within the scope of this invention.

In some embodiments, actual dosage levels of the co-crystal in the pharmaceutical compositions of this invention may be varied so as to obtain an amount of the co-crystal which is effective to achieve the desired therapeutic response for a particular patient, composition, and mode of administration, without being toxic to the patient.

In some embodiments, the selected dosage level will depend upon a variety of factors including the activity of the particular compound of the present invention employed, or the ester, salt or amide thereof, the route of administration, the time of administration, the rate of excretion or metabolism of the particular compound being employed, the rate and extent of absorption, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the composition of the invention employed in the pharmaceutical composition at levels lower than that required in order to achieve the desired therapeutic effect and gradually increase the dosage until the desired effect is achieved.

In some embodiments, a suitable daily dose of a composition of the invention will be that amount of the compound which is the lowest dose effective to produce a therapeutic effect. Such an effective dose will generally depend upon the factors described above. In some embodiments, the compounds are administered at about 0.01 mg/kg to about 200 mg/kg, at about 0.1 mg/kg to about 100 mg/kg, or at about 0.5 mg/kg to about 50 mg/kg. When the compounds described herein are co-administered with another agent (e.g., as sensitizing agents), the effective amount may be less than when the agent is used alone.

In some embodiments, the effective daily dose of the active compound may be administered as two, three, four, five, six or more sub-doses administered separately at appropriate intervals throughout the day, optionally, in unit dosage forms. In some embodiment, dosing is one administration per day.

The description above describes multiple aspects and embodiments of the invention. The patent application specifically contemplates all combinations and permutations of the aspects and embodiments.

### IV. Therapeutic Administration

When used to treat or prevent the above disease or disorders, the compositions disclosed herein may be administered or applied singly, or in combination with other agents. Any of the co-crystals described herein and/or pharmaceutical compositions thereof may also be administered or applied singly, or in combination with other pharmaceutically active agents (e.g., other anti-cancer agents, other arthritis agents, etc.).

The compositions disclosed herein and another therapeutic agent can act additively or synergistically. In one embodiment, the co-crystals disclosed herein and/or a pharmaceutical composition thereof are administered concurrently with the administration of another therapeutic agent. In another embodiment, a co-crystal formulation and/or pharmaceutical composition thereof is administered prior or subsequent to administration of another therapeutic agent.

Sequential or concurrent administration of each therapeutic agent can be effected by any appropriate route including, but not limited to, oral routes, intravenous routes, intramuscular routes, and direct absorption through mucous membrane tissues.

It is understood that the therapeutic agents can be administered by the same route or by different routes. In some embodiments, a first therapeutic agent of the combination selected may be administered by intravenous administration while the other therapeutic agent(s) of the combination may be administered orally. Alternatively, for example, all therapeutic agents may be administered orally or all therapeutic agents may be administered by intravenous injection.

In particular, in one embodiment, the co-crystals disclosed herein and/or pharmaceutical compositions thereof can be used in combination therapy with other chemotherapeutic agents (e.g., alkylating agents (e.g., nitrogen mustards (e.g., cyclophosphamide, ifosfamide, mechlorethamine, melphalen, chlorambucil, hexamethylmelamine, thiotepa), alkyl sulfonates (e.g., busulfan), nitrosoureas, triazines)), antimetabolites (e.g., folic acid analogs, pyrimidine analogs (e.g., fluorouracil, floxuridine, cytosine arabinoside, etc.), purine analogs (e.g., mercaptopurine, thiogunaine, pentostatin, etc.), natural products (e.g., vinblastine, vincristine, etoposide, tertiposide, dactinomycin, daunorubicin, doxorubicin, bleomycin, mithramycin, mitomycin C, L-asparaginase, interferon alpha), platinum coordination complexes (e.g., cis-platinum, carboplatin, etc.), apoptosis inducing agents, glutathione depleting agents or other agents that can alter the redox status of the cell. Those of skill in the art will appreciate that the compositions disclosed herein may also be used in concurrent combination therapy with both the chemotherapeutic agents listed above and radiotherapy.

In some embodiments, the compositions disclosed herein may be administered orally. The compositions disclosed herein may also be administered by any other convenient route, for example, by infusion or bolus injection, by absorption through epithelial or mucocutaneous linings (e.g., oral mucosa, rectal and intestinal mucosa, etc.). Administration can be systemic or local. Various delivery systems are known, (e.g., encapsulation in liposomes, microparticles, microcapsules, capsules, etc.) that can be used to administer the compositions disclosed herein. Methods of administration include, but are not limited to, intradermal, intramuscular, intraperitoneal, intravenous, subcutaneous, intranasal, epidural, oral, sublingual, intranasal, intracerebral, intravaginal, transdermal, rectally, by inhalation, or topically, particularly to the ears, nose, eyes or skin. The mode of administration is left to the discretion of the practitioner and will depend in-part upon the site of the medical condition. In most instances, administration will result in the release of any of the co-crystals described herein and/or pharmaceutical compositions thereof into the bloodstream.

In some embodiments, the compositions disclosed herein may be administered via a medical device, for example, using a medication infusion device, system, and methods as described in International Publication No. WO 2019/241276, the entire disclosure of which is incorporated herein.

In specific embodiments, it may be desirable to administer the compositions disclosed herein locally to the area in need of treatment. This may be achieved, for example, and not by way of limitation, by local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes or fibers. In one embodiment, administration can be by direct injection at the site (or former site) of the disease or disorder.

In certain embodiments, it may be desirable to introduce the compositions disclosed herein into the central nervous system by any suitable route, including intraventricular, intrathecal and epidural injection. Intraventricular injection may be facilitated by an intraventricular catheter, for example, attached to a reservoir, such as an Ommaya reservoir. In some embodiments, the pharmaceutical composition is configured as an inhalable formulation. In some embodiments, the inhalable formulation is configured as a dosage form adapted for pulmonary or nasal administration to the subject. In some embodiments, for example, dosage forms may include those adapted for inhalation such as aerosols and dry powders. In some embodiments, the formulation described herein is suitable for topical delivery to the lung via nose inhalation and/or mouth inhalation. In other embodiments, the compositions disclosed herein may also be administered directly to the lung by inhalation. For administration by inhalation, any of the co-crystals described herein and/or pharmaceutical composition thereof may be conveniently delivered to the lung by a number of different devices.

In some embodiments, the inhalable formulation is configured as an aerosol formulation that comprises a propellant. In some embodiments, the propellant can provide energy to deliver molecules of any of the compounds described herein to the lung. Representative propellants are disclosed in U.S. Pat. No. 6,932,962 B1 and U.S. Pat. No. 8,367,734 B1. In some embodiments, the propellant is presented in the aerosol formulation in an amount ranging from 98% to 99% (w/w) relative to the total weight of the aerosol formulation.

In some embodiments, the aerosol formulation further comprises a surfactant, a co-solvent, and/or a pH buffer. The surfactant can give fine dispersions of the compounds described herein in the propellant and can stabilize the mixture of the compounds described herein in the propellant. In some embodiments, the surfactant comprises a fatty acid or a pharmaceutically acceptable salt thereof, a bile salt, a phospholipid, or an alkyl saccharide. In some embodiments, the surfactant is presented in the formulations described herein in an amount of less than 5% (w/w) (e.g., less than 4%, less than 3%, less than 2%, less than 1% by weight) relative to the total weight of the aerosol formulation.

In some embodiments, the co-solvent can help to stabilize the surfactant and improve the dispersion characteristics. In some embodiments, exemplary co-solvents include ethyl alcohol, isopropyl alcohol, propylene glycol, ethylene glycol, propane, butane, isobutane, pentane, dimethyl ether, diethyl ether and the like. In some embodiments, the co-solvent is present in the formulation in an amount ranging from 0.5% to 20% w/w of the total weight of the formulation. In some embodiments, the co-solvent is present in the formulation in an amount ranging from 0.5% to 5% w/w of the total weight of the formulation. In some embodiments, the co-solvent is present in the formulation in an amount ranging from 0.5% to 1.5% (w/w) of the total weight of the formulation. Representative surfactants, co-solvents, and pH buffers are disclosed in U.S. Pat. No. 6,932,962 B1 and U.S. Pat. No. 8,367,734 B1.

In some embodiments, provided herein are combinations containing the aerosol formulation with the propellant and a pressurized bottle or a nebulizer. In some embodiments, the aerosol formulation with the propellant may be packed in pressurized bottles, where a dosage controller may be used with the pressurized bottle to control the amount of drug being administrated in each spray. In some embodiments, the aerosol formulation with the propellant may be packed in pressurized bottles with a dosage controller, where the dosage controller comprises a valve that controls the delivery of a metered amount of the drug.

In some embodiments, the aerosol formulation is propellant-free and comprises the effective amount of the co-crystal of Compound 1 or the pharmaceutical composition and a solvent. In some embodiments, exemplary solvents include water and alcohols, such as ethanol, isopropanol, and glycols, such as propylene glycol, polyethylene glycol, polypropylene glycol, glycol ether, glycerol and polyoxyethylene alcohols. In some embodiments, the solvent is present in the propellant-free aerosol formulation in an amount ranging from about 0.01% to about 90% (w/w), or about 0.01% to about 50% (w/w), or about 0.01% to about 25% (w/w), or about 0.01% to about 10% (w/w), or about 0.01% to about 5% (w/w) relative to the total weight of the aerosol formulation.

In some embodiments, the propellant-free aerosol formulation may further comprise an emulsifying agent. In some embodiments, exemplary emulsifying agents are disclosed in U.S. Pat. No. 9,498,437 B2. In some embodiments, the emulsifying agent is present in the propellant-free aerosol formulations in an amount ranging from about 0.001% to about 50% (w/w), or about 0.001% to about 25% (w/w), or about 0.001% to about 10% (w/w), or about 0.001% to about 2% (w/w), or about 0.001% to about 1% (w/w) relative to the total weight of the aerosol formulation.

In some embodiments, the propellant-free aerosol formulation may further comprise a complexing agent. In some embodiments, exemplary complexing agents include, but are not limited to, ethylenediaminetetraacetic acid (EDTA) or a pharmaceutically acceptable salt thereof, such as the disodium salt, citric acid, nitrilotriacetic acid and the salts thereof, and sodium edetate. Representative complexing agents are disclosed in U.S. Pat. No. 9,498,437 B2. In some embodiments, the complexing agent is present in the propellant-free aerosol formulations in an amount ranging from about 0.001% to about 50% (w/w), or about 0.001% to about 25% (w/w), or about 0.001% to about 10% (w/w), or about 0.001% to about 2% (w/w), or about 0.001% to about 1% (w/w) relative to the total weight of the aerosol formulation.

In some embodiments, the propellant-free aerosol formulation may further comprise a tonicity agent that can adjust the isotonicity of the present formulations. In some embodiments, exemplary tonicity agents include, but are not limited to, sodium chloride, potassium chloride, zinc chloride, calcium chloride or mixtures thereof. Other osmotic adjusting agents may also include, but are not limited to, mannitol, glycerol, and dextrose or mixtures thereof. Representative tonicity agents are disclosed in U.S. Pat. No. 9,498,437 B2. In some embodiments, the tonicity agent is present in the propellant-free aerosol formulations in an amount ranging from about 0.01% to about 10% (w/w), or about 1% to about 10% (w/w), or about 1% to about 6% (w/w) relative to the total weight of the aerosol formulation. In some embodiments, the aerosol formulation may further comprise the pH buffer.

In some embodiments, provided herein are combinations containing the propellent-free aerosol formulation provided herein and a nebulizer. In some embodiments, the nebulizer can nebulize liquid formulations, including the propellant-free aerosol formulations detailed herein, and produce a nebulized aerosol mist. In some embodiments, the nebulizer may further have an internal baffle, which can selectively remove large droplets from the mist by impaction and allow the droplets to return to the reservoir, so that only fine aerosol droplets are entrained into the lung of the subject by the inhaling air/oxygen. Examples of nebulizers include devices supplied by Sheffield Pharmaceuticals, St. Louis, MO. (Armer et al., U.S. Pat. No. 5,954,047; van der Linden et al., U.S. Pat. No. 5,950,619; van der Linden et al., U.S. Pat. No. 5,970,974) and Batelle Pulmonary Therapeutics, Columbus, OH).

In some embodiments, a Metered Dose Inhaler (“MDI”), which utilizes canisters that contain a suitable low boiling propellant, (e.g., dichlorodifluoromethane, trichlorofluoromethane, dichlorotetrafluoroethane, carbon dioxide or any other suitable gas) may be used to deliver any of the co-crystals described herein and/or pharmaceutical compositions thereof directly to the lung. Specifically, the MDI comprises an aerosol container suitable for containing a propellant-based aerosol formulation and/or a metering valve, for example a side valve, which controls the release of the aerosol formulation to the subject. Representative methods and devices to administer the aerosol formulation with the propellant are disclosed in U.S. Pat. No. 9,498,437 B2.

In another embodiment, a Dry Powder Inhaler (“DPI”) device may be used to administer the compositions disclosed herein to the lung. DPI devices typically use a mechanism such as a burst of gas to create a cloud of dry powder inside a container, which may then be inhaled by the patient and are well known in the art. In a particular embodiment, a popular variation is the multiple dose DPI (“MDDPI”) system, which allows for the delivery of more than one therapeutic dose. MDDPI devices are commercially available from a number of pharmaceutical companies e.g., Schering Plough, Madison, NJ). For example, capsules and cartridges of gelatin for use in an inhaler or insufflator may be formulated containing a powder mix of the compositions disclosed herein and a suitable powder base such as lactose or starch for these systems.

In some embodiments, another type of device that may be used to deliver the compositions disclosed herein to the lung is a liquid spray device supplied, for example, by Aradigm Corporation, Hayward, CA. Liquid spray systems use extremely small nozzle holes to aerosolize liquid drug formulations that may then be directly inhaled into the lung.

In some embodiments, a nebulizer is used to deliver the compositions disclosed herein to the lung. Nebulizers create aerosols from liquid drug formulations by using, for example, ultrasonic energy to form fine particles that may be readily inhaled (see e.g., Verschoyle et al., British J. Cancer, 1999, 80. Suppl. 2, 96). Examples of nebulizers include devices supplied by Sheffield Pharmaceuticals. St. Louis, MO. (Armer et al., U.S. Pat. No. 5,954,047; van der Linden et al., U.S. Pat. No. 5,950,619; van der Linden et al., U.S. Pat. No. 5,970,974) and Batelle Pulmonary Therapeutics, Columbus, OH).

In other embodiments, an electrohydrodynamic (“EHD”) aerosol device is used to deliver the compositions disclosed herein to the lung of a patient. EHD aerosol devices use electrical energy to aerosolize liquid drug solutions or suspensions (see e.g., Noakes et al., U.S. Pat. No. 4,765,539). The electrochemical properties of the formulation may be important parameters to optimize when delivering Compound 1, any of the co-crystals described herein, and/or pharmaceutical composition thereof to the lung with an EHD aerosol device. EHD aerosol devices may more efficiently deliver drugs to the lung than existing pulmonary delivery technologies.

Suitable pharmaceutically acceptable excipients will vary depending upon the particular dosage form chosen. In some embodiments, for example, certain pharmaceutically acceptable excipients may be chosen for their ability to: facilitate the production of aerosol for inhalation, facilitate the production of solution or mist for inhalation, facilitate the production of dry powder for inhalation, or facilitate the production of stable dosage forms.

In some embodiments, the compositions disclosed herein can be delivered in a vesicle, in particular a liposome (e.g., Langer, 1990, Science, 249:1527-1533; Treat et al., in “Liposomes in the Therapy of Infectious Disease and Cancer,” Lopez-Berestein and Fidler (eds.), Liss, New York, pp. 353-365 (1989)).

In some embodiments, the compositions disclosed herein can be delivered via sustained release systems, e.g., oral sustained release systems. In other embodiments, a pump may be used (e.g., Langer, supra, Sefton, 1987, CRC Crit. Ref Biomed. Eng. 14:201; Saudek et al., 1989, N. Engl. J Med. 321:574).

In some embodiments, polymeric materials can be used (e.g., “Medical Applications of Controlled Release,” Langer and Wise (eds.), CRC Press, Boca Raton, Florida (1974); “Controlled Drug Bioavailability,” Drug Product Design and Performance, Smolen and Ball (eds.), Wiley, New York (1984); Ranger et al., 1983, J Macromol. Sci. Rev. Macromol Chem. 23:61; Levy et al., 1985, Science 228: 190 During et al., 1989. Ann. Neurol. 25:351; Howard et al., 1989, J. Neurosurg. 71:105).

In other embodiments, polymeric materials are used for oral sustained release delivery. Polymers include, but are not limited to, sodium carboxymethylcellulose, hydroxypropylcellulose, hydroxypropylmethylcellulose and hydroxyethylcellulose (most preferred, hydroxypropyl methylcellulose). Other cellulose ethers have been described (Alderman, Int. J. Pharm. Tech. & Prod. Mfr. 1984, 5(3) 1-9). Factors affecting drug release are well known to the skilled artisan and have been described in the art (Bamba et al., Int. J. Pharm. 1979, 2, 307).

In other embodiments, enteric-coated preparations can be used for oral sustained release administration. Coating materials include polymers with a pH-dependent solubility (i.e., pH-controlled release), polymers with a slow or pH-dependent rate of swelling, dissolution or erosion (i.e., time-controlled release), polymers that are degraded by enzymes (i.e., enzyme-controlled release) and polymers that form firm layers that are destroyed by an increase in pressure (i.e., pressure-controlled release).

In other embodiments, osmotic delivery systems are used for oral sustained release administration (Verma et al., Drug Dev. Ind. Pharm., 2000, 26:695-708). In some embodiments, OROS™ osmotic devices are used for oral sustained release delivery devices (Theeuwes et al., U.S. Pat. No. 3,845,770; Theeuwes et al., U.S. Pat. No. 3,916,899).

In yet other embodiments, a controlled-release system can be placed in proximity of the target of Compound 1 or any of the co-crystals described herein and/or pharmaceutical composition, thus requiring only a fraction of the systemic dose (e.g., Goodson, in “Medical Applications of Controlled Release,” supra, vol. 2, pp. 115-138 (1984)). Other controlled-release systems previously may also be used (Langer, 1990, Science 249:1527-1533).

In other embodiments, the particles are dispersed in a dedusting additive. For example, the dedusting agent is polyethylene glycol, e.g., PEG-400.

In another aspect, the present invention provides a mixture comprising a composition or a pharmaceutical composition disclosed herein and a blood sample.

In some embodiments, the concentration of Compound 1 is from 0.1 mg/mL of blood to 10 mg/mL of blood, or 0.2 mg/mL of blood to 5 mg/mL of blood, or 0.4 mg/mL of blood to 2.5 mg/mL of blood. Depending upon the circumstances, it may be helpful to administer the co-crystal described herein or a composition comprising the co-crystal such that there is 1 mg of Compound 1 per 2.5 mL of blood.

In some embodiments, suitable methods for the co-crystals described herein are described in, e.g., U.S. Pat. Nos. 10,342,778 or 9,987,270.

### Dose of Co-Crystals Administered

Exemplary dosing amounts of the co-crystals provided herein can be provided with reference to the dosing amounts of Compound 1 in the co-crystals. In some embodiments, the dosing amounts of Compound 1 are provided according to the number of milligrams of Compound 1 to be administered to the patient based on the surface area of the patient as measured in m². In certain embodiments, the dose of Compound 1 administered to the patient is from about 1 mg/m² to about 2 mg/m², about 2 mg/m² to about 4 mg/m², about 4 mg/m² to about 6 mg/m², about 6 mg/m² to about 8 mg/m², about 8 mg/m² to about 10 mg/m², about 10 mg/m² to about 12 mg/m², about 12 mg/m² to about 14 mg/m², about 14 mg/m² to about 16 mg/m², about 16 mg/m² to about 18 mg/m², about 18 mg/m² to about 20 mg/m², about 20 mg/m² to about 25 mg/m², about 25 mg/m² to about 30 mg/m², about 30 mg/m² to about 35 mg/m², about 35 mg/m² to about 40 mg/m², about 40 mg/m² to about 45 mg/m², about 45 mg/m² to about 50 mg/m², about 50 mg/m² to about 60 mg/m², or about 60 mg/m² to about 75 mg/m².

In some embodiments, the dosing amounts of the co-crystal described herein are provided according to the number of milligrams of the co-crystal to be administered to the patient based on the surface area of the patient as measured in m². In certain embodiments, the dose of the co-crystal administered to the patient is from about 1 mg/m² to about 2 mg/m², about 2 mg/m² to about 4 mg/m², about 4 mg/m² to about 6 mg/m², about 6 mg/m² to about 8 mg/m², about 8 mg/m² to about 10 mg/m², about 10 mg/m² to about 12 mg/m², about 12 mg/m² to about 14 mg/m², about 14 mg/m² to about 16 mg/m², about 16 mg/m² to about 18 mg/m², about 18 mg/m² to about 20 mg/m², about 20 mg/m² to about 25 mg/m², about 25 mg/m² to about 30 mg/m², about 30 mg/m² to about 35 mg/m², about 35 mg/m² to about 40 mg/m², about 40 mg/m² to about 45 mg/m², about 45 mg/m² to about 50 mg/m², about 50 mg/m² to about 60 mg/m², about 60 mg/m² to about 75 mg/m², about 75 mg/m² to about 100 mg/m², about 100 mg/m² to about 125 mg/m², about 125 mg/m² to about 150 mg/m², about 150 mg/m² to about 175 mg/m², or about 175 mg/m² to about 200 mg/m².

The dose of the co-crystal administered to the patient may be further characterized according to both the amount of the co-crystal and the mode of delivery, such as intravenous infusion. Accordingly, in certain embodiments, each dose of the formulation comprising the co-crystal is administered to the patient by intravenous infusion providing Compound 1 in an amount ranging from about 1 mg/m² to about 90 mg/m². In certain embodiments, each dose of the co-crystal and/or pharmaceutical formulation comprising the co-crystal is administered to the patient by intravenous infusion providing Compound 1 in an amount ranging from about 1 mg/m² to about 10 mg/m². In certain embodiments, each dose of the co-crystal and/or pharmaceutical formulation comprising the co-crystal is administered to the patient by intravenous infusion providing Compound 1 in an amount ranging from about 1 mg/m² to about 2.5 mg/m². In certain embodiments, each dose of the co-crystal and/or pharmaceutical formulation comprising the co-crystal is administered to the patient by intravenous infusion providing Compound 1 in an amount ranging from about 2.5 mg/m² to about 5 mg/m². In certain embodiments, each dose of the co-crystal and/or pharmaceutical formulation comprising the co-crystal is administered to the patient by intravenous infusion providing Compound 1 in an amount ranging from about 5 mg/m² to about 10 mg/m². In certain embodiments, each dose of the co-crystal and/or pharmaceutical formulation comprising the co-crystal is administered to the patient by intravenous infusion providing Compound 1 in an amount ranging from about 5 mg/m² to about 7 mg/m². In certain embodiments, each dose of the co-crystal and/or pharmaceutical formulation comprising the co-crystal is administered to the patient by intravenous infusion providing Compound 1 in an amount ranging from about 8 mg/m² to about 9 mg/m². In certain embodiments, each dose of the co-crystal and/or pharmaceutical formulation comprising the co-crystal is administered to the patient by intravenous infusion providing Compound 1 in an amount ranging from about 10 mg/m² to about 20 mg/m². In certain embodiments, each dose of the co-crystal and/or pharmaceutical formulation comprising the co-crystal is administered to the patient by intravenous infusion providing Compound 1 in an amount ranging from about 1 mg/m² to about 1.5 mg/m², about 1.5 mg/m² to about 2 mg/m², about 2 mg/m² to about 2.5 mg/m², about 2.5 mg/m² to about 3 mg/m², about 3 mg/m² to about 3.5 mg/m², about 3.5 mg/m² to about 4 mg/m², about 4 mg/m² to about 4.5 mg/m², about 4.5 mg/m² to about 5 mg/m², about 5 mg/m² to about 5.5 mg/m², about 5.5 mg/m² to about 6 mg/m², about 6 mg/m² to about 6.5 mg/m², about 6.5 mg/m² to about 7 mg/m², about 7 mg/m² to about 7.5 mg/m², about 7.5 mg/m² to about 8 mg/m², about 8 mg/m² to about 8.5 mg/m², about 8.5 mg/m² to about 9 mg/m², about 9 mg/m² to about 9.5 mg/m², about 9.5 mg/m² to about 10 mg/m², about 10 mg/m² to about 12 mg/m², about 12 mg/m² to about 14 mg/m², about 14 mg/m² to about 16 mg/m², about 16 mg/m² to about 18 mg/m², about 18 mg/m² to about 20 mg/m², about 20 mg/m² to about 25 mg/m², about 25 mg/m² to about 30 mg/m², about 30 mg/m² to about 35 mg/m², about 35 mg/m² to about 40 mg/m², about 40 mg/m² to about 45 mg/m², or about 45 mg/m² to about 50 mg/m². In certain embodiments, each dose of the co-crystal and/or pharmaceutical formulation comprising the co-crystal is administered to the patient by intravenous infusion providing Compound 1 in an amount ranging from about 3 mg/m² to about 8 mg/m².

In more specific embodiments, each dose of the co-crystal and/or pharmaceutical compositions disclosed herein is administered to the patient by intravenous infusion providing Compound 1 in an amount of about 1.25 mg/m². In certain embodiments, each dose of the co-crystal and/or pharmaceutical compositions disclosed herein is administered to the patient by intravenous infusion providing Compound 1 in an amount of about 2.5 mg/m². In certain embodiments, each dose of the co-crystal and/or pharmaceutical compositions disclosed herein is administered to the patient by intravenous infusion providing Compound 1 in an amount of about 5 mg/m². In certain embodiments, each dose of the co-crystal and/or pharmaceutical compositions disclosed herein is administered to the patient by intravenous infusion providing Compound 1 in an amount of about 8.4 mg/m². In certain embodiments, each dose of the co-crystal and/or pharmaceutical compositions disclosed herein is administered to the patient by intravenous infusion providing Compound 1 in an amount of about 1 mg/m², about 1.5 mg/m², about 2 mg/m², about 2.5 mg/m², about 3 mg/m², about 3.5 mg/m², about 4 mg/m², about 4.5 mg/m², about 5 mg/m², about 5.5 mg/m², about 6 mg/m², about 6.5 mg/m², about 7 mg/m², about 7.5 mg/m², about 8 mg/m², about 8.5 mg/m², about 9 mg/m², about 9.5 mg/m², about 10 mg/m², about 12 mg/m², about 14 mg/m², about 16 mg/m², about 18 mg/m², about 20 mg/m², about 25 mg/m², about 30 mg/m², about 35 mg/m², about 40 mg/m², about 45 mg/m², or about 50 mg/m².

In some embodiments, the method described herein may be further characterized according to the dose of Compound 1 administered to the patient. The dose of Compound 1 described herein for use in combination with temozolomide and the radiation therapy has been selected in view of the dosing schedule and amount of temozolomide and the radiation therapy. Dosing amounts of Compound 1 are provided according to the number of milligrams of Compound 1 to be administered to the patient based on the surface area of the patient as measured in m².

In certain embodiments, each dose of the co-crystals and/or pharmaceutical compositions thereof disclosed herein is administered to the patient by intravenous infusion. In certain embodiments, each dose of the co-crystals and/or pharmaceutical compositions disclosed herein is administered to the patient by intravenous infusion providing Compound 1 in an amount ranging from about 2 mg/m² to about 20 mg/m². In certain embodiments, each dose of the co-crystals and/or pharmaceutical compositions disclosed herein is administered to the patient by intravenous infusion providing Compound 1 in an amount ranging from about 2.5 mg/m² to about 5 mg/m². In certain embodiments, each dose of the co-crystals and/or pharmaceutical compositions disclosed herein is administered to the patient by intravenous infusion providing Compound 1 in an amount ranging from about 5 mg/m² to about 10 mg/m². In certain embodiments, each dose of the co-crystals and/or pharmaceutical compositions disclosed herein is administered to the patient by intravenous infusion providing Compound 1 in an amount ranging from about 10 mg/m² to about 16.5 mg/m². In certain embodiments, each dose of the co-crystals and/or pharmaceutical compositions disclosed herein is administered to the patient by intravenous infusion providing Compound 1 in an amount of about 2.5 mg/m². In certain embodiments, each dose of the co-crystals and/or pharmaceutical compositions disclosed herein is administered to the patient by intravenous infusion providing Compound 1 in an amount of about 5 mg/m². In certain embodiments, each dose of the co-crystals and/or pharmaceutical compositions disclosed herein is administered to the patient by intravenous infusion providing Compound 1 in an amount of about 10 mg/m². In certain embodiments, each dose of the co-crystals and/or pharmaceutical compositions disclosed herein is administered to the patient by intravenous infusion providing Compound 1 in an amount of about 16.5 mg/m².

In certain embodiments, each dose of the co-crystals and/or pharmaceutical compositions disclosed herein is administered to the patient by intravenous infusion providing Compound 1 in an amount of from about 0.1 mg to about 20 mg. In certain embodiments, each dose of the co-crystals and/or pharmaceutical compositions disclosed herein is administered to the patient by intravenous infusion providing Compound 1 in an amount of from about 0.1 mg to about 10 mg. In certain embodiments, each dose of the co-crystals and/or pharmaceutical compositions disclosed herein is administered to the patient by intravenous infusion providing Compound 1 in an amount of from about 0.5 mg to about 4.0 mg. In certain embodiments, each dose of the co-crystals and/or pharmaceutical compositions disclosed herein is administered to the patient by intravenous infusion providing Compound 1 in an amount of about 0.1 mg, 0.2 mg, 0.3 mg, 0.4 mg, 0.5 mg, 1.0 mg, 1.5 mg, 2.0 mg, 2.5 mg, 3.0 mg, 3.5 mg, 4.0 mg, 4.5 mg, 5.0 mg, 7.5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 125 mg, 150 mg, 175 mg, 200 mg, 225 mg or 250 mg. A dose of 0.5 mg to 66 mg of Compound 1 may be administered to a subject, but the actual dose will be determined based on the various factors disclosed herein.

In one approach for administering Compound 1 to a subject as described in the following section, the compound is provided as a liquid formulation containing PEG, for example, PEG-400, at a concentration of 2 mg/mL. In one approach, for each 1 mg of the compound in liquid formulation to be administered the compound is combined with 2.5 mL of blood from the subject to produce a mixture, and then the mixture is administered to the subject. For example, for a dose of 0.5 mg (in 0.25 mL of liquid formulation) is combined with 1.25 mL of blood, or for a dose of 66 mg Compound 1 (in 33 mL of liquid formulation) is combined with 165 mL of blood prior to administration to a subject.

In some embodiments, the amount of a co-crystal and/or pharmaceutical composition thereof administered will, of course, be dependent on, among other factors, the subject being treated, the weight of the subject, the severity of the affliction, the manner of administration and the judgment of the prescribing physician. For example, the dosage of Compound 1 as a co-crystal and/or pharmaceutical formulation may be delivered by a single administration, by multiple applications or controlled release. In some embodiments, dosing may be repeated intermittently, may be provided alone or in combination with other drugs and may continue as long as required for effective treatment of the disease state or disorder.

Suitable dosage ranges for oral administration are dependent on the efficiency of radiosensitization, but are generally about 0.001 mg to about 100 mg of the co-crystal per kg body weight. Dosage ranges may be readily determined by methods known to the artisan of ordinary skill.

Suitable dosage ranges for intravenous (i.v.) administration are about 0.01 mg to about 100 mg per kg/body weight. Suitable dosage ranges for intranasal administration are generally about 0.01 mg/kg body weight to about 1 mg/kg body weight. Suppositories generally contain about 0.01 milligram to about 50 milligrams of a co-crystal per kg/body weight and comprise active ingredient in the range of about 0.5% to about 10% by weight. Recommended dosages for intradermal, intramuscular, intraperitoneal, subcutaneous, epidural, sublingual or intracerebral administration are in the range of about 0.001 mg to about 200 mg per kg/body weight. Effective doses may be extrapolated from dose-response curves derived from in vitro or animal model test systems. Such animal models and systems are well-known in the art.

### Frequency of Administration

In some embodiments, the therapeutically effective amount of the co-crystal of Compound 1 may be administered via a single administration. In some embodiments, the therapeutically effective amount of the co-crystal of Compound 1, may be administered via at least two administrations. In other embodiments, each administration dosage of the therapeutically effective amount of the co-crystal of Compound 1 may be administered at various intervals over a time period until the end of a dosing period. The various intervals are referred to herein as a “frequency.”

In some embodiments, each dose of the therapeutically effective amount of the co-crystal of Compound 1, is administered at a frequency of about 1 dose per 24 hours. In another embodiment, each dose of the therapeutically effective amount of the co-crystal of Compound 1, is administered at a frequency of about 1 dose per 48 hours. In some embodiments, each dose of the therapeutically effective amount of the co-crystal of Compound 1, is administered at a frequency of about 3 doses per week. In another embodiment, each dose of the therapeutically effective amount of the co-crystal of Compound 1, is administered at a frequency of about 2 doses per week. In some embodiments, each dose of the therapeutically effective amount of co-crystal of Compound 1, is administered at a frequency of about 1 dose per week. In other embodiments, each dose of the therapeutically effective amount of the co-crystal of Compound 1, is administered at a frequency of about 2 doses per month. In some embodiments, each dose of the therapeutically effective amount of the co-crystal of Compound 1, is administered at a frequency of about 1 dose per month. In other embodiments, each dose of the therapeutically effective amount of the co-crystal of Compound 1, is administered at a frequency of about 1 dose per 2 months. In some embodiments, each dose of the therapeutically effective amount of the co-crystal of Compound 1, is administered at a frequency of about 1 dose per 3 months.

In other embodiments, each dose of the therapeutically effective amount of the co-crystal of Compound 1, is administered at a frequency of between about 1 dose per day and about 1 dose per week until the end of a dosing period. In some embodiments, each dose of the therapeutically effective amount of the co-crystal of Compound 1, is administered at a frequency of between about 1 dose per day and about 2 doses per week until the end of a dosing period. In other embodiments, each dose of the therapeutically effective amount of the co-crystal of Compound 1, is administered at a frequency of between about 1 dose per week and about 1 dose per month until the end of a dosing period. In other embodiments, each dose of the therapeutically effective amount of the co-crystal of Compound 1, is administered at a frequency of between about 1 dose per month and about 1 dose per 3 months until the end of a dosing period.

Combination with Blood Product and/or Another Agent

In certain embodiments, the pharmaceutical composition for treating the disease, the disorder, or the condition in the subject comprises (1) an effective amount of the co-crystal of Compound 1 and (2) a blood product. In some embodiments, the pharmaceutical composition for treating the disease, the disorder, or the condition in the subject comprises (1) an effective amount of the co-crystal of Compound 1, (2) a blood product, and (3) one or more additional agents. In other embodiments, the pharmaceutical composition for treating the disease, the disorder, or the condition in the subject comprises (1) an effective amount of the co-crystal of Compound 1 and (2) one or more additional agents.

In some embodiments, the blood product comprises erythrocyte cells. In some embodiments, the erythrocyte cells have not undergone any manipulation selected from the group consisting of genetic modification, electroporation, conjugation through biotin, conjugation to a cell-penetrating peptide, conjugation to hemoglobin, dimethyl sulfoxide osmotic pulse, endocytosis and hypotonic preswelling, hypotonic dilution, and hypo-osmotic dialysis. In some embodiments, the blood product is a mixture of packed red blood cells. In other embodiments, the blood product is whole blood. In some embodiments, the whole blood is autologous whole blood.

In some embodiments, each of the one or more additional agents comprise an antioxidant (e.g., lycopene, coenzyme Q10, melatonin, selenium, alpha-lipoic acid, ellagic acid, lutein, resveratrol, anthocyanins, ellagitannins, polyphenols, quercetin, or curcumin), a vitamin (e.g., Vitamin A, Vitamin C, Vitamin E, folic acid, or biopterin), or a mineral (e.g., calcium, phosphorus, potassium, sodium, chloride, magnesium, iron, zinc, iodine, chromium, copper, fluoride, molybdenum, or manganese). In some embodiments, each of the one or more additional agents comprise an anti-inflammatory agent, an anti-diabetic agent, an anti-fibrotic agent, an anti-steatotic agent, a cholesterol/lipid modulating agent, and/or an anti-diabetic agent. In some embodiments, each of the one or more additional agents comprise a sympathomimetic drug such as dobutamine, albuterol, phenylpropanolamine, amphetamine, and ephedrine, and combinations thereof.

In some embodiments, each of the one or more additional agents are selected from the group consisting of: a corticosteroid, human growth hormone, an anabolic steroid, levothyroxine, erythropoietin, selective androgen receptor modulator (SARM), human chorionic gonadotrophin (HCG), adrenocorticotrophin (ACTH), insulin, beta 2 agonist, a beta blocker, a nitrite and/or nitrate, a nitric oxide donor, organic nitrates (e.g., glyceryl trinitrate, isosorbide dinitrate), sodium nitroprusside, sydnonimines (e.g., molsidomine, SIN-1), S-nitrosothiols (e.g., s-nitrosoglutathione, SNAP), and NONOates (e.g., spermine NONOate, DETA-NONOate).

In some embodiments, each of the one or more additional agents are selected from the group consisting of: a PDE5 inhibitor (e.g., sildenafil, tadalafil, vardenafil, or avanafil), a statin, a PCSK9 inhibitor, a small interfering RNA, a selective microsomal triglyceride transfer protein inhibitor, an apolipoprotein B antisense oligonucleotide, a monoclonal antibody against angiopoietin-like protein 3 (ANGPTL3), metformin, aspirin, a peroxisome proliferator-activated receptor (PPAR) agonist, a thyroid hormone receptor beta (THR-β) agonist, insulin, a sulfonylurea, a farnesoid X receptor (FXR) agonist, a glucagon-like peptide 1 (GLP-1) antagonist, a fibric acid derivative, a bile acid sequestrant, nicotinic acid, a selective cholesterol absorption inhibitor, red rice yeast, an omega 3 fatty acid, a fatty acid ester, and an adenosine triphosphate-citrate lyase (ACL) inhibitor.

In some embodiments, each of the one or more additional agents are selected from the group consisting of: a terpene-indole alkaloid compound (e.g., vinorelbine, vinflunine, catharanthine, ajmalicine, vinblastine, vincristine, ajmaline, prajmaline, serpentine, β-yohimbine, and/or reserpine), a bisphosphonate compound (e.g., clodronate, pamidronate, ibandronate, zoledronate, etidronate, olpadronate, and/or alendronate), a glucocorticoid (e.g., methylprednisolone, triamcinolone, triamcinolone acetonide, betamethasone, fluorometholone, fludrocortisone, budesonide, prednisone, prednisolone, dexamethasone, cortisone, and/or hydrocortisone), a coagulation factor (e.g., factor IX and/or factor X), a narcotic (e.g., codeine, fentanyl, hydrocodone, hydromorphone, meperidine, morphine, oxycodone, tramadol, and/or buprenorphine), an opioid receptor agonist, a narcotic receptor antagonist, an anti-viral agent, a biologic response modifier (BRM) agent (e.g., an antigen, a monoclonal antibody, a checkpoint inhibitor, a vaccine, a cytokine, or a CAR-T cell therapy), and an antigen (e.g., neoantigen, a SARS-CoV-2 antigen, a glycoprotein B, a fragment of killed bacteria, and/or a HIV-1 Tat protein). In some embodiments, the anti-viral agent is a nucleoside or nucleotide reverse transcriptase inhibitor, a nonnucleoside reverse transcriptase inhibitor, a protease inhibitor, a fusion inhibitor, a CCR5 co-receptor antagonist, or an HIV integrase strand transfer inhibitor. In some embodiments, the nucleoside analog is saquinavir, indinavir, ritonavir, nelfinavir, lopinavir, fosamprenavir, atazanavir, darunavir, tipranavir, or any combination thereof.

In certain embodiments, the additional agent comprises an anti-cancer agent. In some embodiments, the additional anti-cancer agent is temozolomide, cisplatin, carboplatin, trastuzumab, or sunitinib. In yet other embodiments, the additional anti-cancer agent is temozolomide. In certain embodiments, for any day in which temozolomide is administered to the subject, the temozolomide is administered orally at a dosage of from about 75 mg/m² to about 150 mg/m². Other exemplary anti-cancer agents are presented herein.

In some embodiments, the one or more additional agents in the pharmaceutical composition are subject to a reduced incidence of drug-drug interaction as compared to direct administration of the same additional agent(s) at the same dose without being mixed with the blood product prior to administration. In certain embodiments, the reduced incidence of drug-drug interaction permits the use of a second agent that would have otherwise been contraindicated.

The present invention can provide methods of attenuating interactions of a first drug (e.g., a first therapeutic agent) and a second drug (e.g., a second therapeutic agent) in a mammal. As described herein, interactions of drugs, or drug-drug interactions, can refer to the changes of the effects of a drug or a pharmaceutical composition on a mammal when the pharmaceutical composition is taken together with a second drug or second pharmaceutical composition. In some embodiments, the interactions can occur when more than two drugs are concurrently in a mammal, regardless of the time between the administrations of the two or more drugs and thereby, and react with each other.

In some embodiments, as described herein, “attenuating interactions” of drugs refers to actions that result in reducing or preventing any types of interactions between two or more drugs or reducing the hypersensitivity, the toxicity, or adverse effects that are caused by the interactions of two or more drugs. In some embodiments, the interactions can include, but are not limited to, synergistic or antagonistic interactions. By way of examples, attenuating interactions of the drugs can be at least any one of the following scenarios: reducing and/or preventing drug-drug physical interactions, reducing and/or preventing drug-drug pharmacokinetic interactions, reducing and/or preventing the hypersensitivity caused by co-existence of the drugs, reducing and/or preventing the toxicity caused by co-existence of drugs, or reducing and/or preventing the antagonistic interactions of drugs.

In some embodiments, the effects of the attenuated interactions can be delayed, decreased, or enhanced absorption of either pharmaceutical composition, and thereby decreases or increases the action of either or both therapeutic or diagnostic agents or both pharmaceutical compositions. In some embodiments, the attenuated interactions can impact the transport or the distribution of the therapeutic or diagnostic agents or the pharmaceutical compositions.

Accordingly, in certain embodiments, the subject has reduced incidence and/or severity of side effects compared to subjects receiving a direct administration of the same therapeutic agent at the same dose without being mixed with the blood product prior to administration. In certain embodiments, the subject has reduced side effects compared to subjects receiving a direct administration of the same therapeutic agent at the same dose without being mixed with the blood product prior to administration. In certain embodiments, the dose of the therapeutic agent in the pharmaceutical composition is at least about 10% to about 300% more than the dose recommended for a direct administration of the same therapeutic agent without being mixed with the blood product prior to administration. In certain embodiments, the dose of the therapeutic agent in the pharmaceutical composition is at least 1%, at least 5%, at least 10%, at least 20%, at least 30%, at least 40%, at least 50%, at least 60%, at least 70%, at least 80%, at least 90%, at least 100%, at least 200%, at least 300%, at least 400%, at least 500%, at least 600%, at least 700%, at least 800%, at least 900%, at least 1000%, or higher, inclusive of all ranges and subranges therebetween, more than the dose recommended for a direct administration of the same therapeutic agent without being mixed with the blood product prior to administration.

In certain embodiments, the therapeutic agent has a longer circulating half-life in the subject compared to direct administration of the same therapeutic agent at the same dose without being mixed with the blood product prior to administration. In certain embodiments, the circulating half-life of the therapeutic agent is at least about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 100%, 110%, 110%, 120%, 130%, 140%, 150%, 160%, 170%, 180%, 190%, 200%, 250%, 300%, 350%, 400%, 450%, 500%, 600%, 700%, 800%, 900%, 1000%, or more, longer than the circulating half-life of the same therapeutic agent at the same dose without being mixed with the blood product before administration.

In some embodiments, the side effects/toxicities include, but are not limited to, pulmonary toxicity (e.g., interstitial infiltrates, noncardiogenic pulmonary edema, and/or pulmonary hemorrhage), cardiovascular toxicity (e.g., hypertension), vascular toxicity (e.g., arteriothromboembolic, venous, and/or pericardial effusions), hepatotoxicity (e.g., fatty liver, veno-occlusive disease, pseudocirrhosis, and/or biliary stricture), pancreas toxicity, pancreatitis toxicity, gastrointestinal toxicity (e.g., enteritis, neutropenic colitis, pneumatosis or perforation, megacolon), genitourinary toxicity (e.g., hemorrhagic cystitis and/or neurogenic bladder), peritoneum, mesentery, or soft tissues toxicity (e.g., ascites), and neurologic toxicity (e.g., peripheral neuropathy, and/or central nervous system), ocular toxicity, and ototoxicity (e.g. hearing loss).

### Exemplary Co-Crystal Formulations

The present invention provides co-crystal formulations or formulations comprising one or more co-crystals of Compound 1, e.g., formulations containing whole blood (e.g., autologous or allogeneic blood from the patient being treated), one or more co-crystals of Compound 1, an anticoagulant, and optionally one or more of water, a polyethylene glycol, and N,N-dimethylacetamide. In certain embodiments, the co-crystal formulation comprises whole blood, one or more co-crystal of Compound 1, and an anticoagulant. In certain embodiments, the co-crystal formulation comprises whole blood, one or more co-crystals of Compound 1, an anticoagulant, and optionally one or more of water, a polyethylene glycol, and N,N-dimethylacetamide. In certain embodiments, the co-crystal formulation comprises whole blood, one or more co-crystals of Compound 1, an anticoagulant, and optionally one or more of water, a polyethylene glycol having a number-average molecular weight in the range of about 200 g/mol to about 600 g/mol, and N,N-dimethylacetamide. In certain embodiments, the co-crystal formulation comprises whole blood, one or more co-crystals of Compound 1, an anticoagulant, water, a polyethylene glycol having a number-average molecular weight in the range of about 200 g/mol to about 600 g/mol, and N,N-dimethylacetamide. In certain embodiments, the co-crystal formulation comprises whole blood, one or more co-crystals of Compound 1, an anticoagulant, and optionally one or more of water, a polyethylene glycol having a number-average molecular weight of about 400 g/mol, and N,N-dimethylacetamide. In certain embodiments, the co-crystal formulation comprises whole blood, one or more co-crystals of Compound 1, an anticoagulant, water, a polyethylene glycol having a number-average molecular weight of about 400 g/mol, and N,N-dimethylacetamide.

### Anticoagulant

The formulation of Compound 1 co-crystal may be further characterized according to the identity and/or amount of the anticoagulant. Accordingly, in certain embodiments, the anticoagulant comprises one or more of heparin and a citrate salt. In certain embodiments, the anticoagulant is a solution comprising an alkali metal citrate salt, dextrose, and water. In certain embodiments, the anticoagulant is present in the co-crystal formulation in an amount ranging from about 0.1% wt/wt to about 15% w/w. In certain embodiments, the anticoagulant is present in the Compound 1 co-crystal formulation in an amount ranging from about 1% wt/wt to about 10% w/w. In certain embodiments, the anticoagulant is present in the Compound 1 co-crystal formulation in an amount ranging from about 2% wt/wt to about 8% w/w.

### Amount of Whole Blood in the Compound 1 Co-Crystal Formulation

The formulation of Compound 1 co-crystal may be further characterized according to the amount of whole blood in the Compound 1 co-crystal formulation. Accordingly, in certain embodiments, the whole blood constitutes at least 30% wt/wt of the Compound 1 co-crystal formulation. In certain embodiments, the whole blood constitutes at least 40% wt/wt of the Compound 1 co-crystal formulation. In certain embodiments, the whole blood constitutes at least 50% wt/wt of the Compound 1 co-crystal formulation. In certain embodiments, the whole blood constitutes at least 60% wt/wt of the Compound 1 co-crystal formulation. In certain embodiments, the whole blood constitutes at least 75% wt/wt of the Compound 1 co-crystal formulation. In certain embodiments, the whole blood constitutes at least 90% wt/wt of the Compound 1 co-crystal formulation. In certain embodiments, the whole blood constitutes from about 60% wt/wt to about 99% wt/wt of the Compound 1 co-crystal formulation. In certain embodiments, the whole blood constitutes from about 70% wt/wt to about 95% wt/wt of the Compound 1 co-crystal formulation. In certain embodiments, the whole blood constitutes from about 75% wt/wt to about 90% wt/wt of the Compound 1 co-crystal formulation. In certain embodiments, there is from about 5 mL to about 10 mL of whole blood in the Compound 1 co-crystal formulation, from about 10 mL to about 15 mL of whole blood in the Compound 1 co-crystal formulation, from about 9 mL to about 11 mL of whole blood in the Compound 1 co-crystal formulation, from about 10 mL to about 20 mL of whole blood in the Compound 1 co-crystal formulation, from about 20 mL to about 30 mL of whole blood in the Compound 1 co-crystal formulation, from about 30 mL to about 50 mL of whole blood in the Compound 1 co-crystal formulation, from about 50 mL to about 70 mL of whole blood in the Compound 1 co-crystal formulation, or from about 70 mL to about 90 mL of whole blood in the Compound 1 co-crystal formulation. In certain embodiments, there is from about 90 mL to about 110 mL of whole blood in the Compound 1 co-crystal formulation. In certain embodiments, there is from about 95 mL to about 105 mL of whole blood in the Compound 1 co-crystal formulation. In certain embodiments, there is about 100 mL of whole blood in the Compound 1 co-crystal formulation.

### Volume of Compound 1 Co-Crystal Formulation Administered to a Subject

The method may be further characterized according to the volume of the composition described herein (e.g., an Compound 1 co-crystal formulation) administered to the patient. Accordingly, in certain embodiments, the composition described herein (e.g., an Compound 1 co-crystal formulation) has a volume in the range of about 10 mL to about 200 mL. In certain embodiments, the composition described herein (e.g., an Compound 1 co-crystal formulation) has a volume in the range of about 10 mL to about 15 mL, about 15 mL to about 20 mL, about 20 mL to about 30 mL, or about 30 mL to about 50 mL. In certain embodiments, the composition described herein (e.g., an Compound 1 co-crystal formulation) has a volume in the range of about 50 mL to about 200 mL. In certain embodiments, the composition described herein (e.g., an Compound 1 co-crystal formulation) has a volume in the range of about 75 mL to about 150 mL. In certain embodiments, the composition described herein (e.g., an Compound 1 co-crystal formulation) has a volume in the range of about 90 mL to about 140 mL. In certain embodiments, the composition described herein (e.g., an Compound 1 co-crystal formulation) has a volume in the range of about 100 mL to about 140 mL. In certain embodiments, the composition described herein (e.g., an Compound 1 co-crystal formulation) has a volume in the range of about 100 mL to about 120 mL.

One exemplary more specific formulation is an intravenous formulation containing Compound 1 (e.g., as any of the co-crystals disclosed herein) for intravenous administration to a patient, comprising:
a. whole blood in an amount of at least 60% v/v of the formulation;
b. a polyethylene glycol at a concentration of from about 0.4 μL/mL to about 30 μL/mL in the formulation;
c. N,N-dimethylacetamide at a concentration of from about 0.2 μL/mL to about 15 μL/mL in the formulation;
d. Compound 1 at a concentration of at least 10 g/mL in the co-crystal formulation;
e. water; and
f. an anticoagulant.

Another exemplary more specific formulation is a formulation that comprises:
a. whole blood in an amount of at least 60% v/v of the formulation;
b. a polyethylene glycol at a concentration of from about 0.4 μL/mL to about 30 μL/mL in the formulation;
c. N,N-dimethylacetamide at a concentration of from about 0.2 μL/mL to about 15 μL/mL in the formulation;
d. Compound 1 at a concentration of at least 10 μg/mL in the co-crystal formulation;
e. water; and
f. an anticoagulant.

Another exemplary more specific formulation is a formulation that comprises:
a. whole blood in an amount of at least 60% v/v of the formulation;
b. a polyethylene glycol at a concentration of from about 0.4 μL/mL to about 30 μL/mL in the formulation;
c. N,N-dimethylacetamide at a concentration of from about 0.2 μL/mL to about 15 μL/mL in the formulation;
d. Compound 1 at a concentration of at least 10 μg/mL in the co-crystal formulation;
e. water; and
f. an anticoagulant.

Another exemplary more specific formulation is an intravenous formulation containing Compound 1 (e.g., as any of the co-crystals disclosed herein) for intravenous administration to a patient, comprising:
a. a blood product (e.g., an erythrocyte cell, blood plasma, or whole blood) in an amount of at least 30% v/v of the formulation;
b. optionally a polyethylene glycol at a concentration of from about 0.4 μL/mL to about 30 μL/mL in the formulation;
c. optionally N,N-dimethylacetamide at a concentration of from about 0.2 μL/mL to about 15 μL/mL in the formulation;
d. Compound 1 at a concentration of at least 10 μg/mL in the co-crystal formulation;
e. optionally water; and
f. optionally an anticoagulant.

Another exemplary more specific formulation is an intravenous formulation containing any of the co-crystals disclosed herein, for intravenous administration to a patient, comprising:
a. whole blood in an amount of at least 30% v/v of the formulation;
b. a polyethylene glycol (e.g., at a concentration of from about 0.4 μL/mL to about 30 μL/mL in the formulation);
c. N,N-dimethylacetamide (e.g., at a concentration of from about 0.2 μL/mL to about 15 μL/mL in the formulation);
d. Compound 1 at a concentration of at least 10 μg/mL in the formulation;
e. water; and
f. an anticoagulant.

Another exemplary more specific formulation is a formulation that comprises:
a. whole blood in an amount of at least 30% v/v of the formulation;
b. a polyethylene glycol (e.g., at a concentration of from about 0.4 μL/mL to about 30 μL/mL in the formulation);
c. N,N-dimethylacetamide (e.g., at a concentration of from about 0.2 μL/mL to about 15 μL/mL in the formulation);
d. Compound 1 at a concentration of at least 10 sg/mL in the formulation;
e. water; and
f. an anticoagulant.

### Exemplary Features of Intravenous Formulation

The intravenous formulation may be characterized according to, for example, the identity of a polyethylene glycol, anticoagulant, concentration of Compound 1 (e.g., a crystalline form of Compound 1 as any of the co-crystals disclosed herein), amount of whole blood and other features described herein below.

### Polyethylene Glycol

The formulation may be further characterized according to the identity of a polyethylene glycol in the Compound 1 co-crystal formulation as described herein. Accordingly, in certain embodiments, the polyethylene glycol is a polyethylene glycol having a number-average molecular weight in the range of about 200 g/mol to about 600 g/mol. In certain embodiments, the polyethylene glycol is a polyethylene glycol having a number-average molecular weight of about 400 g/mol.

In certain embodiments, the polyethylene glycol is present at a concentration of from about 0.4 μL/mL to about 4 μL/mL in the formulation. In certain embodiments, the N,N-dimethylacetamide at a concentration of from about 0.2 μL/mL to about 2 μL/mL in the formulation.

### Anticoagulant

The formulation may be further characterized according to the identity of an anticoagulant in the Compound 1 co-crystal formulation as described herein. Accordingly, in certain embodiments, the anticoagulant comprises one or more of heparin and a citrate salt. In certain embodiments, the anticoagulant is a solution comprising an alkali metal citrate salt, dextrose, and water.

### Concentration of Compound 1

The formulation may be further characterized according to the concentration of Compound 1 in the Compound 1 co-crystal formulation as described herein. Accordingly, in certain embodiments, the Compound 1 co-crystal formulation contains Compound 1 at a concentration of at least 20 μg/mL. In certain embodiments, the Compound 1 co-crystal formulation contains Compound 1 at a concentration of at least 50 g/mL. In certain embodiments, the Compound 1 co-crystal formulation contains Compound 1 at a concentration of at least 100 g/mL. In certain embodiments, the Compound 1 co-crystal formulation contains Compound 1 at a concentration of at least 150 g/mL. In certain embodiments, the Compound 1 co-crystal formulation contains Compound 1 at a concentration in the range of about 10 g/mL to about 1 mg/mL. In certain embodiments, the Compound 1 co-crystal formulation contains Compound 1 at a concentration in the range of about 10 μg/mL to about 0.5 mg/mL. In certain embodiments, the Compound 1 co-crystal formulation contains Compound 1 at a concentration in the range of about 10 sg/mL to about 250 μg/mL. In certain embodiments, the Compound 1 co-crystal formulation contains Compound 1 at a concentration in the range of about 20 μg/mL to about 200 μg/mL.

### Amount of Whole Blood

The formulation may be further characterized according to the amount of whole blood in the Compound 1 co-crystal formulation as described herein. Accordingly, in certain embodiments, the whole blood constitutes at least 30% wt/wt of the formulation. In certain embodiments, the whole blood constitutes at least 40% wt/wt of the formulation. In certain embodiments, the whole blood constitutes at least 50% wt/wt of the formulation. In certain embodiments, the whole blood constitutes at least 75% wt/wt of the formulation. In certain embodiments, the whole blood constitutes at least 90% wt/wt of the formulation. In certain embodiments, the whole blood constitutes from about 60% wt/wt to about 99% wt/wt of the formulation. In certain embodiments, the whole blood constitutes from about 70% wt/wt to about 95% wt/wt of the formulation. In certain embodiments, the whole blood constitutes from about 75% wt/wt to about 90% wt/wt of the formulation. In certain embodiments, there is from about 90 mL to about 110 mL of whole blood in the formulation. In certain embodiments, wherein there is from about 95 mL to about 105 mL of whole blood in the formulation. In certain embodiments, there is about 100 mL of whole blood in the formulation.

### Unit Dose Form of Intravenous Formulation

The formulation may be further characterized according to the volume of a unit dose of the co-crystal formulation as described herein. Accordingly, in certain embodiments, the formulation is in the form of a unit dose having a volume in the range of about 10 mL to about 200 mL. In certain embodiments, the formulation is in the form of a unit dose having a volume in the range of about 10 mL to about 15 mL, about 15 mL to about 20 mL, about 20 mL to about 30 mL, about 30 mL to about 40 mL, or about 40 mL to about 50 mL. In certain embodiments, the formulation is in the form of a unit dose having a volume in the range of about 50 mL to about 200 mL. In certain embodiments, the formulation is in the form of a unit dose having a volume in the range of about 75 mL to about 150 mL. In certain embodiments, the formulation is in the form of a unit dose having a volume in the range of about 90 mL to about 140 mL. In certain embodiments, the formulation is in the form of a unit dose having a volume in the range of about 100 mL to about 140 mL. In certain embodiments, the formulation is in the form of a unit dose having a volume in the range of about 100 mL to about 120 mL.

### Characterization of Pain Effect Upon Intravenous Administration to a Subject

The formulation as described herein may be further characterized according to the extent of pain experienced by the patient upon intravenous administration of the Compound 1 co-crystal formulation to the patient. Accordingly, in certain embodiments, the formulation is characterized by the feature that any pain experienced by the patient at the site of intravenous administration due to intravenous administration of the formulation to the patient at a rate in the range of 10 mL/hour to 50 mL/hour is no greater than Grade 2. In certain embodiments, wherein the formulation is characterized by the feature that any pain experienced by the patient at the site of intravenous administration due to intravenous administration of the formulation to the patient at a rate in the range of 10 mL/hour to 50 mL/hour is no greater than Grade 1.

The description above describes multiple aspects and embodiments of the invention. The patent application specifically contemplates all combinations and permutations of the aspects and embodiments.

### V. Therapeutic Applications

In one aspect, provided are methods of using any of the co-crystals or compositions described herein to treat, prevent, or mitigate any of the diseases, the disorders, or the conditions described herein in a subject. In some aspect, provided are uses of any of the co-crystals or compositions described herein in the manufacture of a medicament to treat, prevent, or mitigate any of the diseases, the disorders, or the conditions described herein. In some aspect, provided are the co-crystals and compositions for use in the treatment, prevention, or mitigation of any of the diseases, the disorders, or the conditions described herein. In some embodiments, the disease is associated with abnormal cell proliferation. In some embodiments, the disease, the disorder, or the condition is selected from the group consisting of: muscular dystrophies, patent foramen ovale, obesity, mitochondrial disorders or diseases, chronic obstructive pulmonary disease, hyperCKemia, motor neuron disease, neuromuscular disease, multiple sclerosis, Charcot-myositis including polymyositis and dermatomyositis, insulin resistance, myoedema, rhabdomyolysis, idiopathic chronic muscle fatigue, reduced skeletal muscle function, and disrupted skeletal muscle function or metabolism.

In one aspect, provided are methods of treating or preventing a cancer. In some embodiments, the method comprises administering to a subject in need thereof an effective amount of any of the co-crystal or composition described herein. In some embodiments, the subject is mammal. In some embodiments, the subject has a locally advanced solid tumor including a gastrointestinal malignancy, head and neck cancer, gynecological cancer, breast cancer, hepatocellular cancer, esophageal cancer, lung cancer, genitourinary cancer, gastrointestinal tract cancers, genitourinary cancers, hepatocellular carcinoma, glioblastoma, or sarcoma. In some embodiments, the subject has an injury that includes include mucositis, dysphagia, dyspepsia, laryngeal inflammation, oral dysesthesia, vomiting, salivary duct inflammation, esophagitis, any gastrointestinal distress, myelosuppression, impotence, infertility, dermatitis, hair loss or increased creatinine. In some embodiments, the subject is human. In some embodiments, the administration is performed intravenously or orally.

In another aspect, provided are methods of treating or preventing an ischemic or hypoxic condition. In some embodiments, the ischemic condition is an acute or chronic ischemic condition. In other embodiments, the acute ischemic condition is myocardial infarction, ischemic stroke, pulmonary embolism, perinatal hypoxia, circulatory shock, mountain sickness or acute respiratory failure. In certain embodiments, the chronic ischemic condition is atherosclerosis, chronic venous insufficiency, chronic heart failure, cardiac cirrhosis, macular degeneration, sleep apnea, Raynaud's disease, systemic sclerosis, nonbacterial thrombotic endocarditis, occlusive artery disease, angina pectoris, transient ischemic attacks, or chronic alcoholic liver disease. In some embodiments, the hypoxic condition is cancer, gastric or duodenal ulcers, liver or renal disease, thrombocytopenia, a blood coagulation disorder, a chronic illness, a therapeutic intervention that produces anemia such as cancer chemotherapy or altitude sickness.

In some embodiments, the method comprises administering to a subject in need thereof an effective amount of any of the co-crystal or composition described herein. In some embodiments, the subject is mammal. In some embodiments, the subject is human. In some embodiments, the administration is performed intravenously or orally.

In another aspect, provided are methods of treating or preventing neurodegenerative, allergic, autoimmune, fibrotic, inflammatory, infectious, pulmonary, cardiac, vascular, or metabolic diseases. In some embodiments, the disease is associated with abnormal cell proliferation, such as an inflammatory disease (e.g., arthritis, diabetic retinopathy, rheumatoid arthritis, neovascular glaucoma, or psoriasis), a cardiovascular disease (e.g., arteriosclerosis, pulmonary hypertension, systemic hypertension, angina, Cardiac Syndrome X, myocardial infarction, peripheral artery disease, or Raynaud's disease), or an autoimmune disease. In some embodiments where the disease is pulmonary hypertension, the pulmonary hypertension is associated with congenital left to right intracardiac shunts, portal hypertension, persistent pulmonary hypertension of the newborn, collagen vascular diseases, HIV infection, an exposure to drugs, and/or an exposure to toxins. In some embodiments, the disorder is a reproductive-related disorder, such as endometriosis or fibroids. In some embodiments, the method comprises subcutaneously administering to a subject in need thereof an effective amount of any of the co-crystal or composition described herein. In some embodiments, the subject is mammal. In some embodiments, the subject is human.

In another aspect, provided are methods of protecting against normal tissue toxicity caused by chemotherapy and/or radiation therapy. In some embodiments, the method comprises subcutaneously administering to a subject in need thereof an effective amount of any of the co-crystal or composition described herein before the subject is exposed to the chemotherapy and/or radiation therapy. In some embodiments, the subject is mammal. In some embodiments, the subject is human.

In another aspect, provided are methods of treating a patient suffering from reduced blood volume or low perfusion. In some embodiments, the method comprises administering to a patient in need thereof a blood product comprising any of the co-crystal or composition described herein. In some embodiments, the subject is mammal. In some embodiments, the subject is human.

In some embodiments, the method generally involves administering to a patient in need of such treatment or prevention a therapeutically effective amount of a composition disclosed herein. In certain circumstances, Compound 1 is intracellularly activated by the reducing environment of a tumor cell. In other circumstances, the subject is irradiated to activate Compound 1. Without wishing to be bound by theory, irradiation or reduction of Compound 1 may lead to formation of free radicals that subsequently prevent cell replication and kill cells, presumably by interfering with DNA replication and/or reacting with cell membranes. However, other mechanisms, presently unknown, may account for the efficacy of Compound 1 in treating or preventing abnormal cell proliferation. Under other circumstances, Compound 1 may be activated by both intracellular reduction and external irradiation. In these embodiments, a synergistic or additive effect may be observed.

In another aspect, the present invention provides a method treating diseases or disorders characterized by abnormal cell proliferation in a subject in need thereof, the method comprising administering to the subject an effective amount of the composition described herein, the pharmaceutical composition described herein or the mixture described herein, thereby to treat the abnormal cell proliferation in the subject.

In some embodiments, the diseases or disorders characterized by abnormal cell proliferation are inflammation, cardiovascular disease and autoimmune disease. In some embodiments, the inflammatory disease is arthritis, diabetic retinopathy, diabetes, rheumatoid arthritis, neovascular glaucoma and psoriasis. In some embodiments, the cardiovascular disease is arteriosclerosis, pulmonary hypertension, systemic hypertension, angina, Cardiac Syndrome X, myocardial infarction, peripheral artery disease, or Raynaud's disease.

In another aspect, the present invention provides a method treating cancer in a subject in need thereof, the method comprising administering to the subject an effective amount of the composition described herein, the pharmaceutical composition described herein or the mixture described herein, thereby to treat the cancer in the subject.

In some embodiments, the cancers are vascularized solid tumor cancers, including but not limited to, carcinomas of the lung, breast, ovary, stomach, pancreas, larynx, esophagus, testes, liver, parotid, biliary tract, colon, rectum, cervix, uterus, endometrium, kidney, bladder, prostrate, thyroid, squamous cell carcinomas, adenocarcinomas, small cell carcinomas, melanomas, gliomas, including, but not limited to, astrocytomas, glioblastomas; neuroblastomas, sarcomas, including, but not limited to, angiosarcomas, chondrosarcomas.

In another aspect, the present invention provides a method for treating a hemolytic condition in a subject in need thereof, the method comprising administering to the subject an effective amount of the composition described herein, the pharmaceutical composition described herein or the mixture described herein, thereby to treat the hemolytic condition in the subject.

In some embodiments, the hemolytic condition is sickle cell disease. In other embodiments, the hemolytic condition is selected from one of the following exemplary hemolytic conditions including sickle cell crisis, thalassemia, hemoglobin C disease, hemoglobin SC disease, sickle thalassemia, hereditary spherocytosis, hereditary elliptocytosis, hereditary ovalocytosis, glucose-6-phosphate deficiency and other red blood cell enzyme deficiencies, paroxysmal nocturnal hemoglobinuria (PNH), paroxysmal cold hemoglobinuria (PCH), thrombotic thrombocytopenic purpura/hemolytic uremic syndrome (TTP/HUS), idiopathic autoimmune hemolytic anemia, drug-induced immune hemolytic anemia, secondary immune hemolytic anemia, non-immunehemolytic anemia caused by chemical or physical agents, malaria, falciparum malaria, bartonellosis, babesiosis, clostridial infection, severe Haemophilus influenzae type b infection, extensive burns, transfusion reaction, rhabdomyolysis (myoglobinemia), transfusion of aged blood, cardiopulmonary bypass, and hemodialysis. See Howard J. Sickle cell disease and other hemoglobinopathies. In: Goldman L, Schafer A I, eds. Goldman-Cecil Medicine. 26th ed. Philadelphia, PA: Elsevier: 2020: chap 154; and K. Smith-Whitley, et al. Hemoglobinopathies. In: Kliegman R M, St. Geme J W, Blum N J, Shah S S, Tasker R C, Wilson K M. eds. Nelson Textbook of Pediatrics. 21st ed. Philadelphia, PA: Elsevier; 2020: chap 489.

In another embodiment, a method for treating a disease or a disorder associated with dyslipidemia in a subject in need thereof is described. The method includes administering to the subject an effective amount of the pharmaceutical composition described herein to treat the disease or the disorder associated with the dyslipidemia in the subject. In some embodiments, the disease or the disorder is selected from the group consisting of: non-alcoholic fatty liver disease (NAFLD), non-alcoholic steatohepatitis (NASH), atherosclerosis, hyperlipidemia, hypercholesterolemia, steatosis, liver fibrosis, coronary heart disease, peripheral vascular disease, stroke, aortic aneurysm, glucose intolerance, and diabetes.

In some embodiments, the composition described herein or the pharmaceutical composition described herein is combined with blood harvested from the subject to create a mixture, whereupon the mixture is administered to the subject. In some embodiments, the blood is whole blood, e.g., autologous or allogeneic whole blood. In other embodiments the blood is a blood product, including but not limited to one or more of plasma, erythrocytes. In another embodiment, the composition described herein or the pharmaceutical composition described herein is combined with blood product for donation to a patient suffering from reduced blood volume or low perfusion, who is suffering from hemorrhagic shock. In another embodiment, the composition described herein or the pharmaceutical composition described herein is administered to the patient separately from the blood product.

In another aspect, the present invention provides a method for in-vitro sterilization. Biological solutions may be treated with the invention, which are toxic to pathogenic bacteria, viruses and cells. This process can also be catalyzed by the application of external energy such as light and heat.

In another aspect, the present invention provides a method of treatment for a patient suffering from a bacterial infection. In some embodiments, the bacterial infection may be a gram-positive bacterial infection or a gram negative bacterial infection. In certain embodiments, the bacterial infection is a gram-positive cocci bacterial infection or a gram-positive bacilli bacterial infection. In certain other embodiments, the bacterial infection is a gram-negative bacterial infection. In certain other embodiments, the bacterial infection is a gram-negative cocci bacterial infection or a gram negative bacilli bacterial infection.

The type of bacterial infection can also be characterized according to whether the bacterial infection is caused by anaerobic or aerobic bacteria. In certain embodiments, the bacterial infection is an anaerobic bacterial infection. In certain other embodiments, the bacterial infection is an aerobic bacterial infection.

In certain embodiments, the bacterial infection is a mycobacterial infection. In more particular embodiments, the bacterial infection is an infection of bacteria selected from the group comprising Mycobacterium tuberculosis, Staphylococcus aureus, Staphylococcus epidermidis, Enterococcus faecalis, Enterococcus faecium, Streptococcus pneunoniae, Streptococcus pyogenes, Mycobacterium smegmatis, Bacillus anthracis, Escherichia coli, Proteus mirabilis, Pseudomonas aeruginosa, Acinetobacter baumannii, Yersinia enterocolitica, Francisella tularensis, Eubacterium lentum, Bacteroides fragilis, Fusobacterium nucleatum, Porphyromonas asaccharolyticus, Clostridium perfringens, and Clostridium difficile. In still other embodiments the bacterial infection is an infection of Mycobacterium tuberculosis bacteria (abbreviated as “MTB” or “TB”).

In certain other embodiments, the bacterial infection is due to a member of the genus Peptostreptococci, a Peptostreptococci asaccharolyticus, a Peptostreptococci magnus, a Peptostreptococci micros, a Peptostreptococci prevotii, a member of the genus Porphyromonas, a Porphyromonas asaccharolytica, a Porphyromonas canoris, a Porphyromonas gingivalis, a Porphyromonas macaccae, a member of the genus Actinomyces, an Actinomyces israelii, an Actinomyces odontolyticus, a member of the genus Clostridium, a Clostridium innocuum, a Clostridium clostridioforme, a Clostridium difficile, a member of the genus Anaerobiospirillum, a member of the genus Bacteroides, a Bacteroides tectum, a Bacteroides ureolyticus, a Bacteroides gracilis (Campylobacter gracilis), a member of the genus Prevotella, a Prevotella intermedia, a Prevotella heparinolytica, a Prevotella orisbuccae, a Prevotella bivia, a Prevotella melaninogenica, a member of the genus Fusobacterium, a Fusobacterium naviforme, a Fusobacterium necrophorum, a Fusobacterium varium, a Fusobacterium ulcerans, a Fusobacterium russii, a member of the genus Bilophila or a Bilophila wadsworthia.

In certain other embodiments, the bacterial infection is due to an antibiotic-resistant bacteria, both aerobic and anaerobic, Gram positive and Gram negative.

In another aspect, the present invention provides a method of treating or preventing an ischemic or hypoxic condition in a subject in need thereof, the method comprising administering to the subject a therapeutically effective amount of any of the co-crystals or pharmaceutical compositions described herein or a mixture described herein. In some embodiments, the subject is a mammal.

In some embodiments, the ischemic condition is an acute or chronic ischemic condition. In other embodiments, the acute ischemic condition is myocardial infarction, ischemic stroke, pulmonary embolism, perinatal hypoxia, circulatory shock, mountain sickness or acute respiratory failure. In certain embodiments, the chronic ischemic condition is atherosclerosis, chronic venous insufficiency, chronic heart failure, cardiac cirrhosis, diabetes, macular degeneration, sleep apnea, Raynaud's disease, systemic sclerosis, nonbacterial thrombotic endocarditis, occlusive artery disease, angina pectoris, transient ischemic attacks, or chronic alcoholic liver disease. In some embodiments, the hypoxic condition is cancer, gastric or duodenal ulcers, liver or renal disease, thrombocytopenia, a blood coagulation disorder, a chronic illness, a therapeutic intervention that produces anemia such as cancer chemotherapy or altitude sickness. In other embodiments, the cancer is bladder cancer, breast cancer, clear cell kidney cancer, head/neck squamous cell carcinoma, lung squamous cell carcinoma, malignant melanoma, colorectal cancer, head and neck cancer, cervical cancer, non-small-cell lung cancer (NSCLC), ovarian cancer, pancreatic cancer, prostate cancer, renal cell cancer, small-cell lung cancer (SCLC), triple negative breast cancer, acute lymphoblastic leukemia (ALL), acute myeloid leukemia (AML), chronic lymphocytic leukemia (CLL), chronic myeloid leukemia (CML), diffuse large B-cell lymphoma (DLBCL), EBV-positive DLBCL, primary mediastinal large B-cell lymphoma, T-cell/histiocyte-rich large B-cell lymphoma, follicular lymphoma, Hodgkin's lymphoma (HL), mantle cell lymphoma (MCL), multiple myeloma (MM), myeloid cell leukemia-1 protein (Mcl-1), myelodysplasia syndrome (MDS), non-Hodgkin's lymphoma (NHL), or small lymphocytic lymphoma (SLL).

Additional medical conditions contemplated for treatment or prevention using compositions described herein include nitrogen oxide related rheumatoid arthritis, diabetes (including neuropathies and vasculopathies), and systemic lupus erythematosus.

A contemplated pharmaceutical composition may comprise at least 0.5 mg of Compound 1 as a co-crystal and is administered intravenously, nasally, optically, intraperitoneally, subcutaneously, or orally.

### Type of Cancer

When a composition disclosed herein is being administered to a subject suffering from cancer in order to treat the cancer, the method may be further characterized according to type of cancer to be treated. For example, in certain embodiments, the cancer is a solid tumor. For example, the cancer can brain cancer, bladder cancer, breast cancer, cervical cancer, cholangiocarcinoma, colon cancer, colorectal cancer, endometrial cancer, esophageal cancer, lung cancer, liver cancer, melanoma, ovarian cancer, pancreatic cancer, prostate cancer, rectal cancer, renal cancer, stomach cancer, testicular cancer, or uterine cancer.

In certain embodiments, the cancer is brain cancer. In certain embodiments, the cancer is colorectal cancer. In certain embodiments, the cancer is cholangiocarcinoma or lung cancer. In certain embodiments, the cancer is lung cancer. In certain embodiments, the lung cancer is small cell lung cancer. In certain other embodiments, the cancer is non-small cell lung cancer. In certain embodiments, the cancer is a leukemia or lymphoma. In certain embodiments, the cancer is a B-cell lymphoma or non-Hodgkin lymphoma.

Additional exemplary cancers for treatment include, for example, bladder cancer, breast cancer, cervical cancer, colon cancer, colorectal cancer, endometrial cancer, esophageal cancer, leukemia, lung cancer, liver cancer, melanoma, ovarian cancer, pancreatic cancer, prostate cancer, rectal cancer, renal cancer, stomach cancer, testicular cancer, and uterine cancer.

It is contemplated that the cancer can be a vascularized tumor, squamous cell carcinoma, adenocarcinoma, small cell carcinoma, melanoma, glioma, neuroblastoma, sarcoma (e.g., an angiosarcoma or chondrosarcoma), larynx cancer, parotid cancer, biliary tract cancer, thyroid cancer, acral lentiginous melanoma, actinic keratoses, acute lymphocytic leukemia, acute myeloid leukemia, adenoid cystic carcinoma, adenomas, adenosarcoma, adenosquamous carcinoma, anal canal cancer, anal cancer, anorectum cancer, astrocytic tumor, bartholin gland carcinoma, basal cell carcinoma, biliary cancer, bone cancer, bone marrow cancer, bronchial cancer, bronchial gland carcinoma, carcinoid, cholangiocarcinoma, chondosarcoma, choroid plexus papilloma/carcinoma, chronic lymphocytic leukemia, chronic myeloid leukemia, clear cell carcinoma, connective tissue cancer, cystadenoma, digestive system cancer, duodenum cancer, endocrine system cancer, endodermal sinus tumor, endometrial hyperplasia, endometrial stromal sarcoma, endometrioid adenocarcinoma, endothelial cell cancer, ependymal cancer, epithelial cell cancer, Ewing's sarcoma, eye and orbit cancer, female genital cancer, focal nodular hyperplasia, gallbladder cancer, gastric antrum cancer, gastric fundus cancer, gastrinoma, glioblastoma, glucagonoma, heart cancer, hemangiblastomas, hemangioendothelioma, hemangiomas, hepatic adenoma, hepatic adenomatosis, hepatobiliary cancer, hepatocellular carcinoma, Hodgkin's disease, ileum cancer, insulinoma, intaepithelial neoplasia, interepithelial squamous cell neoplasia, intrahepatic bile duct cancer, invasive squamous cell carcinoma, jejunum cancer, joint cancer, Kaposi's sarcoma, pelvic cancer, large cell carcinoma, large intestine cancer, leiomyosarcoma, lentigo maligna melanomas, lymphoma, male genital cancer, malignant melanoma, malignant mesothelial tumors, medulloblastoma, medulloepithelioma, meningeal cancer, mesothelial cancer, metastatic carcinoma, mouth cancer, mucoepidermoid carcinoma, multiple myeloma, muscle cancer, nasal tract cancer, nervous system cancer, neuroepithelial adenocarcinoma nodular melanoma, non-epithelial skin cancer, non-Hodgkin's lymphoma, oat cell carcinoma, oligodendroglial cancer, oral cavity cancer, osteosarcoma, papillary serous adenocarcinoma, penile cancer, pharynx cancer, pituitary tumors, plasmacytoma, pseudosarcoma, pulmonary blastoma, rectal cancer, renal cell carcinoma, respiratory system cancer, retinoblastoma, rhabdomyosarcoma, sarcoma, serous carcinoma, sinus cancer, skin cancer, small cell carcinoma, small intestine cancer, smooth muscle cancer, soft tissue cancer, somatostatin-secreting tumor, spine cancer, squamous cell carcinoma, striated muscle cancer, submesothelial cancer, superficial spreading melanoma, T cell leukemia, tongue cancer, undifferentiated carcinoma, ureter cancer, urethra cancer, urinary bladder cancer, urinary system cancer, uterine cervix cancer, uterine corpus cancer, uveal melanoma, vaginal cancer, verrucous carcinoma, VIPoma, vulva cancer, well differentiated carcinoma, or Wilms tumor.

The invention also provides therapeutic methods for treating brain metastases. For example, the methods may use a particular dosing regimen of Compound 1, radiation therapy, and optionally an additional anti-cancer agent. The therapeutic method can be further characterized according to type of brain metastasis to be treated. For example, the brain metastasis can be characterized according to the type of primary tumor from which the brain metastasis results. In certain embodiments, the brain metastasis is a brain metastasis from a melanoma, lung cancer, breast cancer, colon cancer, kidney cancer, liver cancer, ovarian cancer, pancreatic cancer, prostate cancer, rectal cancer, stomach cancer, testicular cancer, uterine cancer, endometrial cancer, or esophageal cancer. In certain other embodiments, the brain metastasis is a brain metastasis from a melanoma, lung cancer, breast cancer, colon cancer, or kidney cancer. In yet other embodiments, the brain metastasis is from a melanoma.

Without limitation, exemplary cancers from which a brain metastasis may result include, for example, bladder cancer, breast cancer, cervical cancer, colon cancer, colorectal cancer, endometrial cancer, esophageal cancer, leukemia, lung cancer, liver cancer, melanoma, ovarian cancer, pancreatic cancer, prostate cancer, rectal cancer, renal cancer, stomach cancer, testicular cancer, and uterine cancer. In yet other embodiments, the cancer is a vascularized tumor, squamous cell carcinoma, adenocarcinoma, small cell carcinoma, melanoma, glioma, neuroblastoma, sarcoma (e.g., an angiosarcoma or chondrosarcoma), larynx cancer, parotid cancer, biliary tract cancer, thyroid cancer, acral lentiginous melanoma, actinic keratoses, acute lymphocytic leukemia, acute myeloid leukemia, adenoid cystic carcinoma, adenomas, adenosarcoma, adenosquamous carcinoma, anal canal cancer, anal cancer, anorectum cancer, astrocytic tumor, bartholin gland carcinoma, basal cell carcinoma, biliary cancer, bone cancer, bone marrow cancer, bronchial cancer, bronchial gland carcinoma, carcinoid, cholangiocarcinoma, chondosarcoma, choroid plexus papilloma/carcinoma, chronic lymphocytic leukemia, chronic myeloid leukemia, clear cell carcinoma, connective tissue cancer, cystadenoma, digestive system cancer, duodenum cancer, endocrine system cancer, endodermal sinus tumor, endometrial hyperplasia, endometrial stromal sarcoma, endometrioid adenocarcinoma, endothelial cell cancer, ependymal cancer, epithelial cell cancer, Ewing's sarcoma, eye and orbit cancer, female genital cancer, focal nodular hyperplasia, gallbladder cancer, gastric antrum cancer, gastric fundus cancer, gastrinoma, glioblastoma, glucagonoma, heart cancer, hemangiblastomas, hemangioendothelioma, hemangiomas, hepatic adenoma, hepatic adenomatosis, hepatobiliary cancer, hepatocellular carcinoma, Hodgkin's disease, ileum cancer, insulinoma, intaepithelial neoplasia, interepithelial squamous cell neoplasia, intrahepatic bile duct cancer, invasive squamous cell carcinoma, jejunum cancer, joint cancer, Kaposi's sarcoma, pelvic cancer, large cell carcinoma, large intestine cancer, leiomyosarcoma, lentigo maligna melanomas, lymphoma, male genital cancer, malignant melanoma, malignant mesothelial tumors, medulloblastoma, medulloepithelioma, meningeal cancer, mesothelial cancer, metastatic carcinoma, mouth cancer, mucoepidermoid carcinoma, multiple myeloma, muscle cancer, nasal tract cancer, nervous system cancer, neuroepithelial adenocarcinoma nodular melanoma, non-epithelial skin cancer, non-Hodgkin's lymphoma, oat cell carcinoma, oligodendroglial cancer, oral cavity cancer, osteosarcoma, papillary serous adenocarcinoma, penile cancer, pharynx cancer, pituitary tumors, plasmacytoma, pseudosarcoma, pulmonary blastoma, rectal cancer, renal cell carcinoma, respiratory system cancer, retinoblastoma, rhabdomyosarcoma, sarcoma, serous carcinoma, sinus cancer, skin cancer, small cell carcinoma, small intestine cancer, smooth muscle cancer, soft tissue cancer, somatostatin-secreting tumor, spine cancer, squamous cell carcinoma, striated muscle cancer, submesothelial cancer, superficial spreading melanoma, T cell leukemia, tongue cancer, undifferentiated carcinoma, ureter cancer, urethra cancer, urinary bladder cancer, urinary system cancer, uterine cervix cancer, uterine corpus cancer, uveal melanoma, vaginal cancer, verrucous carcinoma, VIPoma, vulva cancer, well differentiated carcinoma, or Wilms tumor.

Combinations with Other Additional Anti-Cancer Agents

In certain embodiments, the method described herein further comprises administering an additional anti-cancer agent to the subject. In certain embodiments, the additional anti-cancer agent is temozolomide, cisplatin, carboplatin, trastuzumab, or sunitinib. In yet other embodiments, the additional anti-cancer agent is temozolomide. In certain embodiments, for any day in which temozolomide is administered to the patient, the temozolomide is administered orally at a dosage of from about 75 mg/m² to about 150 mg/m².

Further exemplary additional anti-cancer agents include, for example, azacitidine, azathioprine, bleomycin, capecitabine, carmustine, chlorambucil, cyclophosphamide, cytarabine, dacarbazine, daunorubicin, docetaxel, doxifluridine, doxorubicin, epirubicin, epothilone, etoposide, fluorouracil, fulvestrant, gemcitabine, hydroxyurea, idarubicin, imatinib, lomustine, mechlorethamine, mercaptopurine, methotrexate, mitoxantrone, oxaliplatin, paclitaxel, pemetrexed, procarbazine, raloxifene, teniposide, thiotepa, tioguanine, tamoxifen, toremifene, valrubicin, vinblastine, vincristine, vindesine, vinorelbine, and pharmaceutically acceptable salts thereof.

In yet other embodiments, the additional anti-cancer agent is abraxane; acivicin; aclarubicin; acodazole hydrochloride; acronine; adozelesin; aldesleukin; altretamine; ambomycin; ametantrone acetate; amrubicin; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefmgol; celecoxib; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; de/.aguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; elsamitrucin; enloplatm; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine; fosquidone; fostriecin sodium; gemcitabine; gemcitabine hydrochloride; herceptin; hydroxyurea; idarubicin hydrochloride; ifosfamide; ilmofosine; iproplatin; irinotecan; irinotecan hydrochloride; lanreotide acetate; lapatinib; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium porfiromycin; prednimustine; procarbazine hydrochloride; puromycin; puromycin hydrochloride; pyrazofurin; riboprine; romidepsin; safingol; safingol hydrochloride; semustine; simtrazene; sparfosate sodium; sparsomycin; spirogermanium hydrochloride; spiromustine; spiroplatin; a stem cell treatment; streptonigrin; streptozocin; sulofenur; talisomycin; tecogalan sodium; taxotere; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; or zorubicin hydrochloride.

### Characterization of Anti-Cancer Effects

When a composition described herein is being administered to a cancer patient in order to treat the cancer, the therapeutic method may be further characterized according to the anti-cancer effect of the treatment, such as (i) a reduction in the size of at least one tumor in the patient, and/or (ii) reduction in the number of tumors in the patient.

Accordingly, in certain embodiments, the therapeutic method described herein is characterized by at least a 20% reduction in the size of at least one tumor in the patient. In certain other embodiments, there is at least a 35% reduction in the size of at least one tumor in the patient. In certain other embodiments, there is at least a 50% reduction in the size of at least one tumor in the patient. In certain other embodiments, there is at least a 60%, 70%, 80% or 90% reduction in the size of at least one tumor in the patient. In certain other embodiments, there is about a 5%-50%, 10%-50%, 20%-50%, 5%-75%, 10%-75%, 20%-75%, or 50%-90% reduction in the size of at least one tumor in the patient.

When the cancer to be treated are brain metastases, the method may be further characterized according to the reduction in number and/or size of the brain metastases. In certain embodiments, there is at least a 20% reduction in the number of brain metastases in the patient. In certain other embodiments, there is at least a 35% reduction in the number of brain metastases in the patient. In yet other embodiments, there is at least a 50% reduction in the number of brain metastases in the patient. In certain other embodiments, there is at least a 60%, 70%, 80% or 90% reduction in the number of brain metastases in the patient. In certain other embodiments, there is about a 5%-50%, 10%-50%, 20%-50%, 5%-75%, 10%-75%, 20%-75%, or 50%-90% reduction in the number of brain metastases in the patient.

### Subjects/Patients for Treatment

The therapeutic method may be further characterized according to the patient to be treated. In certain embodiments, the patient is an adult human. In certain other embodiments, the patient is a pediatric human.

In certain embodiments, the patient does not suffer from anemia or have reduced blood volume. In certain embodiments, the patient has at least 95% of the amount of their average daily blood volume.

### Tissue Protection

Under certain circumstances, it is contemplated that the compounds described herein can be used to protect against normal tissue toxicity that may occur when a subject is undergoing chemotherapy and/or radiation. The method comprising: subcutaneously administering to a subject in need thereof an effective amount of the impact-insensitive composition, pharmaceutical composition, or mixture that contains any of the co-crystals described herein and/or pharmaceutical compositions thereof as described herein before the subject is exposed to the chemotherapy and/or radiation therapy.

Under certain circumstances, the subject has cancer, for example head and neck cancer. Also, it is contemplated that at least about 0.5 mg (for example, 0.5 mg to 4 mg) of any of the co-crystals described herein and/or pharmaceutical compositions thereof is administered to the subject, which can be administered, for example, by injection, in single doses or via multiple divided doses injections.

Under certain circumstance, the normal tissue toxicity to be protected against can be acute mucositis (for example, late mucositis) or dysphagia.

### VI. Kits for Use in Medical Applications

The current invention also provides therapeutic kits comprising one or more co-crystals described herein and/or pharmaceutical compositions thereof. The therapeutic kits may also contain other compounds (e.g., chemotherapeutic agents, natural products, apoptosis-inducing agents, etc.) or pharmaceutical compositions thereof.

Therapeutic kits may have a single container which contains one or more co-crystals described herein and/or pharmaceutical compositions thereof with or without other components (e.g., other compounds or pharmaceutical compositions of these other compounds) or may have distinct container for each component. In some embodiments, therapeutic kits include one or more co-crystals described herein and/or a pharmaceutical composition thereof packaged for use in combination with the co-administration of a second compound (e.g., a chemotherapeutic agent, a natural product, an apoptosis-inducing agent, etc.) or a pharmaceutical composition thereof. The components of the kit may be pre-complexed or each component may be in a separate distinct container prior to administration to a patient.

The components of the kit may be provided in one or more liquid solutions, e.g., an aqueous solution or a sterile aqueous solution. The components of the kit may also be provided as solids, which may be converted into liquids by addition of suitable solvents, which may be provided in another distinct container.

The container of a therapeutic kit may be a vial, test tube, flask, bottle, syringe, or any other means of enclosing a solid or liquid. Usually, when there is more than one component, the kit will contain a second vial or other container, which allows for separate dosing. The kit may also contain another container for a pharmaceutically acceptable liquid.

In some embodiments, a therapeutic kit will contain apparatus (e.g., one or more needles, syringes, eye droppers, pipette, etc.), which enables administration of the components of the kit.

### EXAMPLES

The presently disclosed subject matter will be better understood by reference to the following Examples, which are provided as exemplary of the invention, and not by way of limitation.

Table A below provides acronyms for the analytical techniques used. Details for these techniques can be found in the Instrumental section below.

TABLE A
Acronyms for the analytical techniques used.

| Analytical Technique Acronym | Full Description |
|---|---|
| DSC | Differential scanning calorimetry |
| DVS | Dynamic vapor sorption |
| FT-Raman | Fourier transform Raman |
| IR | Infrared |
| LF-Raman | Low frequency Raman |
| NMR | Nuclear magnetic resonance |
| TGA | Thermogravimetric analysis |
| XRPD | X-ray powder diffraction |

### Instrumental

### X-Ray Powder Diffraction (XRPD)

The Rigaku Smart-Lab X-ray diffraction system was configured for reflection Bragg-Brentano geometry using a line source X-ray beam. The x-ray source is a Cu Long Fine Focus tube that was operated at 40 kV and 44 ma.

Powder samples were prepared in a low background Si holder using light manual pressure to keep the sample surfaces flat and level with the reference surface of the sample holder. Each sample was analyzed from 2 to 40° 2θ using a continuous scan of 6° 2θ per minute with an effective step size of 0.02° 2θ.

It is possible to analyze the materials using a similar XRPD system equipped with a Cu x-ray source, using a Bragg-Brentano geometry and step size of 0.02° 20. It is possible to obtain similar quality XRPD diffractograms by varying incident/receiving slits, step sizes and scan speed.

### Differential Scanning Calorimetry (DSC)

DSC analyses were carried out using a TA Instruments Q2500 Discovery Series instrument. The instrument temperature calibration was performed using indium. The DSC cell was kept under a nitrogen purge of ˜50 mL per minute during each analysis. The sample (about 1-2 mg) was placed in a standard, crimped, aluminum pan and was heated from 25° C. to 350° C. at a rate of 10° C. per minute. The type of pan, preparation of the pan for analysis, and heating rate can impact the outcome of the analysis results. Nitrogen purge rate can be varied as appropriate for the specific instrument specifications.

### Thermogravimetric Analysis (TGA)

The TG analysis was carried out using a TA Instruments Q5500 Discovery Series instrument. The instrument balance was calibrated using class M weights and the temperature calibration was performed using alumel. The nitrogen purge was ˜40 mL per minute at the balance and ˜60 mL per minute at the furnace. Each sample (about 2-5 mg) was placed into a pre-tared platinum pan and heated from 20° C. to 350° C. at a rate of 10° C. per minute. The heating rate can impact the outcome of the analysis results. Nitrogen purge rate can be varied as appropriate for the specific instrument specifications.

### Dynamic Vapor Sorption (DVS) Analysis

DVS analysis was carried out using a TA Instruments Q5000 Dynamic Vapor Sorption analyzer. The instrument was calibrated with standard weights and a sodium bromide standard for humidity. Approximately 20 mg of sample was loaded into a metal-coated quartz pan for analysis. The sample was analyzed at 25° C. with a maximum equilibration time of one hour in 10% relative humidity (RH) steps from 5 to 95% RH (adsorption cycle) and from 95 to 5% RH (desorption cycle). The movement from one step to the next occurred either after satisfying the equilibrium criterion of 0.01% weight change or, if the equilibrium criterion was not met, after one hour. The temperature for the DVS analysis can impact the outcome of the results.

### Infrared (IR) Spectroscopy

IR spectra were acquired using a Thermo Scientific model iS50 Fourier-transform (FT) IR spectrophotometer equipped with a deuterated triglycine sulfate (DTGS) detector, a potassium bromide (KBr) beamsplitter, and a Polaris™ long-life IR source. A diamond attenuated total reflectance (ATR) sampling accessory with a spectral range of 4000 cm-1 to 400 cm-1 was used. Each spectrum was the result of 128 co-added scans acquired at 2 cm-1 resolution. A single beam background scan of air was acquired before the sample scan, allowing presentation of the spectra in log l/R units. Wavelength calibration was performed using polystyrene.

### Raman Spectroscopy

Fourier transform (FT) Raman spectra were acquired on a Nicolet model 6700 spectrometer interfaced to a Nexus Raman accessory module. This instrument is configured with a Nd:YAG laser operating at 1024 nm, a CaF₂ beamsplitter, and a indium gallium arsenide detector. Samples were packed into a 3-inch glass NMR tube for analysis. The FT-Raman spectrum was collected with 256 signal-averaged scans at a resolution of 4 cm⁻¹ over the spectral range 3700-100 cm⁻¹.

### Low Frequency (LF) Raman Spectroscopy

Low frequency Raman spectra were obtained using a Renishaw inVia Raman microscope equipped with an Ondax THz-Raman system (TR-PROBE; excitation laser 853.1 nm, notch filter). Individual sample powders were analyzed in the open air using the probe tip attachment. Spectra were acquired using a static scan centered at 36 cm⁻¹ to collect over the spectral range −575 to 575 cm⁻¹ with 100% power, an exposure time of one second, and 32 accumulations. The wavelength calibration was confirmed using a sulfur reference standard.

### Nuclear Magnetic Resonance (NMR) Spectroscopy

The ¹H NMR spectra were acquired on a Bruker Avance II 400 spectrometer utilizing a 5-mm cryoprobe operating at an observing frequency of 400.18 MHz. Samples were prepared by dissolving material in DMSO-d₆. The solutions were filtered and placed into individual 5-mm NMR tubes for subsequent spectral acquisition at 295K.

### Example 1: Preparation of Compound 1 Calcium Chloride Co-Crystal

In a 1-dram glass vial were combined 200.3 mg of Compound 1 and 83.3 mg of calcium chloride (1 molar equivalent). Two mL of 95:5 ethanol:water were added to the vial and the resulting slurry was stirred magnetically at room temperature for 11 days. The slurry was then vacuum filtered and the solids were allowed to dry at room temperature.

Compound 1 calcium chloride co-crystal was analyzed by XRPD, DSC, TGA, DVS, IR spectroscopy, FT-Raman spectroscopy, LF Raman spectroscopy, and NMR spectroscopy. The solid resulting from the DVS analysis was also) analyzed by XRPD. Characterization data are summarized below in Table 1A.

TABLE 1A

| Sample | Analytical | |
|---|---|---|
| Information | Technique | Results |
| Compound 1 | XRPD | Calcium chloride co-crystal |
| Calcium chloride | DSC | Endo 112.3, 136.2° C. |
| co-crystal | TGA | 9.3% start to 130° C. |
| | DVS | Very hygroscopic |
| | Post-DVS | Unchanged |
| | XRPD | |
| | IR | — |
| | FT-Raman | — |
| | LF-Raman | — |
| | NMR | Consistent with structure |

Compound 1 calcium chloride co-crystal is crystalline based on XRPD data where the sample shows very sharp and well resolved signals as provided in FIG. 1A, as well as in Tables 1B to 1D below.

TABLE 1B
X-ray powder diffraction full peak listing and relative
intensities of Compound 1 calcium chloride co-crystal

| | Relative | d-spacing |
|---|---|---|
| °2θ | Intensity | value (Å) |
| 10.8 | 10 | 8.1615 |
| 11.6 | 9 | 7.6547 |
| 12.9 | 29 | 6.8731 |
| 14.9 | 16 | 5.9455 |
| 17.7 | 53 | 5.0220 |
| 18.0 | 26 | 4.9225 |
| 18.2 | 8 | 4.8689 |
| 18.9 | 6 | 4.6953 |
| 19.2 | 5 | 4.6273 |
| 19.9 | 78 | 4.4660 |
| 20.7 | 9 | 4.2868 |
| 21.5 | 100 | 4.1406 |
| 21.9 | 25 | 4.0621 |
| 22.9 | 12 | 3.8834 |
| 25.0 | 40 | 3.5673 |
| 25.9 | 6 | 3.4452 |
| 26.7 | 27 | 3.3387 |
| 27.1 | 42 | 3.2903 |
| 27.7 | 70 | 3.2204 |
| 28.3 | 9 | 3.1535 |
| 29.0 | 14 | 3.0831 |
| 29.1 | 14 | 3.0727 |
| 29.9 | 31 | 2.9883 |
| 30.4 | 37 | 2.9402 |
| 31.0 | 17 | 2.8829 |
| 31.8 | 10 | 2.8122 |
| 32.2 | 27 | 2.7782 |
| 32.5 | 12 | 2.7565 |
| 32.9 | 9 | 2.7207 |
| 33.4 | 11 | 2.6858 |
| 33.6 | 12 | 2.6656 |
| 34.2 | 24 | 2.6247 |
| 34.7 | 16 | 2.5837 |
| 35.5 | 28 | 2.5300 |
| 35.6 | 32 | 2.5232 |
| 35.8 | 30 | 2.5082 |
| 36.7 | 11 | 2.4474 |
| 37.0 | 9 | 2.4308 |
| 37.5 | 8 | 2.3995 |
| 38.0 | 16 | 2.3691 |
| 39.0 | 18 | 2.3083 |

TABLE 1C
X-ray powder diffraction selected peak listing and relative
intensities of Compound 1 calcium chloride co-crystal

| | Relative |
|---|---|
| °2θ ± 0.2 °2θ | Intensity |
| 10.8 | 10 |
| 11.6 | 9 |
| 12.9 | 29 |
| 14.9 | 16 |

TABLE 1D
X-ray powder diffraction unique peak and relative
intensity of Compound 1 calcium chloride co-crystal

| | Relative |
|---|---|
| °2θ ± 0.2 °2θ | Intensity |
| 10.8 | 10 |
| 11.6 | 9 |
| 14.9 | 16 |

Compound 1 calcium chloride co-crystal is stable under various humidity conditions. It is very hygroscopic based on the DVS data provided in FIG. 1D, with a weight gain of over 25% up to 95% RH (relative humidity). As shown by the XRPD data provided in FIG. 1E, the crystalline form remained unchanged after the DVS analysis where the sample was exposed up to 95% RH, and down to 5% RH.

FIG. 1F shows the IR spectrum of Compound 1 Calcium chloride co-crystal, and Table 1E below provides IR peaks.

TABLE 1E
Complete IR spectrum peak list of Compound
1 Calcium chloride co-crystal

| IR Peak | IR Peak |
|---|---|
| (cm⁻¹) | (cm⁻¹) |
| 411 | 1214 |
| 450 | 1253 |
| 510 | 1285 |
| 551 | 1308 |
| 568 | 1339 |
| 654 | 1369 |
| 708 | 1414 |
| 724 | 1438 |
| 757 | 1462 |
| 790 | 1567 |
| 841 | 1586 |
| 871 | 1644 |
| 897 | 1679 |
| 926 | 2956 |
| 1009 | 3005 |
| 1097 | 3020 |
| 1116 | 3387 |
| 1191 | |

FIG. 1G shows the FT-Raman spectrum of Compound 1 Calcium chloride co-crystal, and Tables 1F and 1G below provides FT-Raman peaks.

TABLE 1F
Complete FT-Raman peak listing of Compound
1 Calcium chloride co-crystal

| Raman Shift | Raman Shift |
|---|---|
| (cm⁻¹) | (cm⁻¹) |
| 122 | 1098 |
| 161 | 1123 |
| 190 | 1189 |
| 257 | 1214 |
| 282 | 1252 |
| 309 | 1273 |
| 340 | 1287 |
| 413 | 1308 |
| 448 | 1341 |
| 512 | 1370 |
| 570 | 1414 |
| 656 | 1461 |
| 710 | 1569 |
| 724 | 1590 |
| 757 | 1680 |
| 791 | 2866 |
| 841 | 2961 |
| 870 | 2999 |
| 897 | 3019 |
| 1009 | |

TABLE 1G
Selected FT-Raman peak listing of Compound 1
Calcium chloride co-crystal
Raman Shift (cm⁻¹) ± 2 cm⁻¹
257
413
448
791

FIG. 1H shows the Low Frequency (LF) Raman Spectrum of Compound 1 Calcium chloride co-crystal, and Table 1H below provides LF-Raman peaks.

TABLE 1H
Complete LF-Raman peak listing of Compound 1
Calcium chloride co-crystal
Raman Shift (cm⁻¹)

−340
−309
−282
−256
−189
−121
−76
−46
−35

Compound 1 Calcium chloride co-crystal is unsolvated based on its ¹H NMR data provided in FIG. 1I.

### Example 2: Preparation of Compound 1 Pyridoxine HCl Co-Crystal

In a 1-dram glass vial were combined 199.6 mg of Compound 1 and 152.6 mg of pyridoxine HCl (1 molar equivalent). Two mL of 95:5 ethanol:water were added to the vial and the resulting slurry was stirred magnetically at room temperature for 11 days. The slurry was then vacuum filtered and the solids were allowed to dry at room temperature.

Compound 1 pyridoxine HCl co-crystal was analyzed by XRPD, DSC, TGA, DVS, IR spectroscopy, FT-Raman spectroscopy, LF Raman spectroscopy, and NMR spectroscopy. The solid resulting from the DVS analysis was also analyzed by XRPD. Characterization data are summarized below in Table 2A

TABLE 2A

| Sample | Analytical | |
|---|---|---|
| Information | Technique | Results |
| Compound 1 | XRPD | Pyridoxine HCl Co-crystal |
| pyridoxine HCl | DSC | Endo 134.7° C. |
| co-crystal | TGA | 0.3% start to 130° C. |
| | DVS | Slightly hygroscopic |
| | Post-DVS | Unchanged |
| | XRPD | |
| | IR | — |
| | Raman | — |
| | LF-Raman | — |
| | NMR | Consistent with structure |
| | | Stoichiometry: 1:1 |

Compound 1 pyridoxine HCl co-crystal is crystalline based on XRPD data where the sample shows very sharp and well resolved signals as provided in FIG. 2A, as well as in Tables 2B to 2D below.

TABLE 2B
X-ray powder diffraction full peak listing and relative
intensities of Compound 1 pyridoxine HCl co-crystal

| | Relative | d-spacing |
|---|---|---|
| °2θ | Intensity | value (Å) |
| 10.3 | 40 | 8.5881 |
| 10.7 | 5 | 8.2680 |
| 10.9 | 6 | 8.1019 |
| 11.4 | 5 | 7.7483 |
| 12.7 | 10 | 6.9701 |
| 14.8 | 6 | 5.9935 |
| 15.6 | 6 | 5.6730 |
| 17.0 | 18 | 5.2277 |
| 17.5 | 29 | 5.0619 |
| 17.9 | 20 | 4.9607 |
| 18.7 | 10 | 4.7551 |
| 19.7 | 42 | 4.4973 |
| 20.0 | 10 | 4.4438 |
| 20.3 | 12 | 4.3660 |
| 20.8 | 55 | 4.2745 |
| 21.3 | 60 | 4.1752 |
| 21.6 | 27 | 4.1066 |
| 21.8 | 17 | 4.0694 |
| 22.6 | 7 | 3.9377 |
| 22.8 | 10 | 3.9069 |
| 23.3 | 14 | 3.8144 |
| 24.2 | 21 | 3.6836 |
| 24.8 | 29 | 3.5957 |
| 25.0 | 76 | 3.5561 |
| 25.8 | 42 | 3.4478 |
| 26.5 | 12 | 3.3585 |
| 26.9 | 29 | 3.3192 |
| 27.5 | 35 | 3.2434 |
| 27.8 | 100 | 3.2045 |
| 28.2 | 12 | 3.1644 |
| 28.7 | 19 | 3.1147 |
| 29.8 | 26 | 3.0020 |
| 30.2 | 15 | 2.9612 |
| 30.8 | 9 | 2.8993 |
| 31.5 | 7 | 2.8418 |
| 32.0 | 19 | 2.8002 |
| 32.2 | 7 | 2.7765 |
| 32.9 | 10 | 2.7223 |
| 33.8 | 22 | 2.6503 |
| 34.4 | 16 | 2.6040 |
| 35.3 | 23 | 2.5425 |
| 35.5 | 23 | 2.5287 |
| 37.0 | 22 | 2.4295 |
| 37.7 | 12 | 2.3836 |
| 38.1 | 8 | 2.3595 |
| 38.8 | 12 | 2.3197 |

TABLE 2C
X-ray powder diffraction selected peak listing and relative
intensities of Compound 1 pyridoxine HCl co-crystal

| | Relative |
|---|---|
| °2θ ± 0.2 °2θ | Intensity |
| 10.7 | 5 |
| 11.4 | 5 |
| 12.7 | 10 |
| 14.8 | 6 |

TABLE 2D
X-ray powder diffraction unique peak and relative
intensity of Compound 1 HCl co-crystal

| | Relative |
|---|---|
| °2θ ± 0.2 °2θ | Intensity |
| 11.4 | 5 |
| 12.7 | 10 |
| 14.8 | 6 |

Compound 1 pyridoxine HCl co-crystal is stable under various humidity conditions. It is slightly hygroscopic based on the DVS data with a weight gain of approximately 0.5% up to 95% RH (relative humidity). The crystalline form remained unchanged after the DVS analysis where the sample was exposed up to 95% RH, and down to 5% RH.

FIG. 2F shows the IR spectrum of Compound 1 pyridoxine HCl co-crystal, and Table 2E below provides IR peaks.

TABLE 2E
Complete IR spectrum peak list of Compound
1 pyridoxine HCl co-crystal

| IR Peak | IR Peak |
|---|---|
| (cm⁻¹) | (cm⁻¹) |
| 409 | 1368 |
| 450 | 1382 |
| 475 | 1400 |
| 511 | 1413 |
| 575 | 1439 |
| 589 | 1447 |
| 622 | 1460 |
| 653 | 1480 |
| 685 | 1545 |
| 724 | 1567 |
| 749 | 1587 |
| 791 | 1625 |
| 841 | 1645 |
| 870 | 1679 |
| 926 | 1732 |
| 962 | 1805 |
| 989 | 1826 |
| 1017 | 1900 |
| 1089 | 1929 |
| 1115 | 2817 |
| 1191 | 2955 |
| 1214 | 3093 |
| 1253 | 3232 |
| 1275 | 3267 |
| 1307 | 3317 |
| 1339 | |

FIG. 2G shows the FT-Raman spectrum of Compound 1 pyridoxine HCl co-crystal, and Tables 2F and 2G below provides FT-Raman peaks.

TABLE 2F
Complete FT-Raman peak listing of Compound
1 pyridoxine HCl co-crystal

| Raman Shift | Raman Shift |
|---|---|
| (cm⁻¹) | (cm⁻¹) |
| 116 | 1088 |
| 161 | 1123 |
| 257 | 1216 |
| 286 | 1233 |
| 309 | 1293 |
| 329 | 1322 |
| 356 | 1356 |
| 404 | 1356 |
| 477 | 1370 |
| 517 | 1381 |
| 533 | 1449 |
| 568 | 1569 |
| 654 | 1588 |
| 691 | 1628 |
| 724 | 1644 |
| 753 | 1678 |
| 791 | 2826 |
| 841 | 2868 |
| 868 | 2901 |
| 897 | 2934 |
| 928 | 2959 |
| 965 | 2999 |
| 988 | 3015 |
| 1009 | 3102 |
| 1052 | |

TABLE 2G
Selected FT-Raman peak listing of Compound 1
pyridoxine HCl co-crystal
Raman Shift (cm⁻¹) ± 2 cm⁻¹

477
791
1233
1628
1644

FIG. 2H shows the Low Frequency (LF) Raman Spectrum of Compound 1 pyridoxine HCl co-crystal, and Table 2H below provides LF-Raman peaks.

TABLE 2H
Complete LF-Raman peak listing of Compound 1
pyridoxine HCl co-crystal
Raman Shift (cm⁻¹)

−329
−285
−167
−118
−83
−57

Compound 1 pyridoxine HCl co-crystal is unsolvated and consistent with a 1:1 co-crystal based on its ¹H NMR data, provided in FIG. 2I.

### Example 3: Preparation of Compound 1 Thiamine HCl Co-Crystal

In a 1-dram glass vial were combined 200.2 mg of Compound 1 and 251.8 mg of thiamine HCl (1 molar equivalent). Two mL of 95:5 ethanol:water were added to the vial and the resulting slurry was stirred magnetically at room temperature for 11 days. The slurry was then vacuum filtered and the solids were allowed to dry at room temperature.

Compound 1 thiamine HCl co-crystal was analyzed by XRPD, DSC, TGA, DVS, IR spectroscopy, FT-Raman spectroscopy, LF Raman spectroscopy, and NMR spectroscopy. The solid resulting from the DVS analysis was also analyzed by XRPD. Characterization data are summarized below in Table 3A.

TABLE 3A

| Sample | Analytical | |
|---|---|---|
| Information | Technique | Results |
| Compound 1 | XRPD | Thiamine HCl Co-crystal |
| thiamine HCl | DSC | Endo 131.8° C. |
| co-crystal | TGA | 1.3% start to 130° C. |
| | DVS | moderately hygroscopic |
| | Post-DVS | Unchanged |
| | XRPD | |
| | IR | — |
| | Raman | — |
| | LF-Raman | — |
| | NMR | Consistent with structure |
| | | Stoichiometry: 1:1 |

Compound 1 thiamine HCl co-crystal is crystalline based on XRPD data where the sample shows very sharp and well resolved signals as provided in FIG. 3A, as well as in Tables 3B to 3D below.

TABLE 3B
X-ray powder diffraction full peak listing and relative
intensities of Compound 1 thiamine HCl co-crystal

| | Relative | d spacing |
|---|---|---|
| °2θ | Intensity | value (Å) |
| 7.5 | 9 | 11.7245 |
| 8.2 | 24 | 10.7560 |
| 10.3 | 32 | 8.5550 |
| 10.7 | 7 | 8.2680 |
| 11.9 | 5 | 7.4492 |
| 12.7 | 7 | 6.9920 |
| 14.8 | 11 | 5.9855 |
| 15.0 | 8 | 5.9139 |
| 16.4 | 33 | 5.3984 |
| 17.2 | 26 | 5.1672 |
| 17.5 | 32 | 5.0676 |
| 17.9 | 36 | 4.9607 |
| 18.2 | 8 | 4.8742 |
| 18.9 | 22 | 4.7051 |
| 19.3 | 24 | 4.5941 |
| 19.7 | 69 | 4.4973 |
| 20.6 | 19 | 4.3115 |
| 21.3 | 100 | 4.1713 |
| 21.6 | 54 | 4.1216 |
| 22.7 | 20 | 3.9103 |
| 23.0 | 32 | 3.8601 |
| 23.5 | 16 | 3.7919 |
| 23.6 | 15 | 3.7666 |
| 24.1 | 34 | 3.6866 |
| 24.7 | 53 | 3.6072 |
| 25.0 | 9 | 3.5617 |
| 25.3 | 13 | 3.5202 |
| 25.6 | 10 | 3.4796 |
| 25.9 | 31 | 3.4452 |
| 26.7 | 56 | 3.3411 |
| 27.5 | 83 | 3.2480 |
| 27.7 | 32 | 3.2181 |
| 28.1 | 28 | 3.1710 |
| 28.8 | 19 | 3.1041 |
| 29.2 | 8 | 3.0603 |
| 29.7 | 42 | 3.0059 |
| 30.1 | 52 | 2.9669 |
| 30.8 | 15 | 2.9030 |
| 31.1 | 26 | 2.8774 |
| 31.8 | 32 | 2.8105 |
| 32.1 | 20 | 2.7849 |
| 32.5 | 15 | 2.7565 |
| 32.7 | 15 | 2.7369 |
| 33.2 | 14 | 2.7000 |
| 33.7 | 31 | 2.6626 |
| 34.3 | 19 | 2.6129 |
| 34.8 | 10 | 2.5793 |
| 35.4 | 42 | 2.5384 |
| 35.7 | 14 | 2.5150 |
| 36.1 | 12 | 2.4853 |
| 36.4 | 16 | 2.4669 |
| 37.2 | 10 | 2.4169 |
| 37.7 | 14 | 2.3885 |
| 38.8 | 15 | 2.3232 |
| 39.2 | 10 | 2.2958 |

TABLE 3C
X-ray powder diffraction selected peak listing and relative
intensities of Compound 1 thiamine HCl co-crystal

| | Relative |
|---|---|
| °2θ ± 0.2 °2θ | Intensity |
| 7.5 | 9 |
| 8.2 | 24 |
| 10.3 | 32 |
| 14.8 | 11 |
| 16.4 | 33 |

TABLE 3D
X-ray powder diffraction unique peak and relative
intensity of Compound 1 thiamine HCl co-crystal

| | Relative |
|---|---|
| °2θ ± 0.2 °2θ | Intensity |
| 8.2 | 24 |
| 10.3 | 32 |
| 16.4 | 33 |

Compound 1 thiamine HCl co-crystal is stable under various humidity conditions. It is moderately hygroscopic based on the DVS data with a weight gain of approximately 2% up to 95% RH (relative humidity). The crystalline form remained unchanged after the DVS analysis where the sample was exposed up to 95% RH, and down to 5% RH.

FIG. 3F shows the IR spectrum of Compound 1 thiamine HCl co-crystal, and Table 3E below provides IR peaks.

TABLE 3E
Complete IR spectrum peak list of
Compound 1 thiamine HCl co-crystal

| IR Peak | IR Peak |
|---|---|
| (cm⁻¹) | (cm⁻¹) |
| 408 | 1213 |
| 440 | 1225 |
| 450 | 1233 |
| 463 | 1251 |
| 509 | 1286 |
| 542 | 1308 |
| 572 | 1339 |
| 645 | 1368 |
| 702 | 1380 |
| 724 | 1399 |
| 751 | 1438 |
| 760 | 1480 |
| 790 | 1508 |
| 841 | 1532 |
| 870 | 1567 |
| 891 | 1587 |
| 972 | 1613 |
| 1007 | 1655 |
| 1026 | 2915 |
| 1045 | 2964 |
| 1072 | 3038 |
| 1095 | 3193 |
| 1115 | 3414 |
| 1170 | 3498 |
| 1189 | |

FIG. 3G shows the FT-Raman spectrum of Compound 1 thiamine HCl co-crystal, and Tables 3F and 3G below provides FT-Raman peaks.

TABLE 3F
Complete FT-Raman peak listing of
Compound 1 thiamine HCl co-crystal

| Raman Shift | Raman Shift |
|---|---|
| (cm⁻¹) | (cm⁻¹) |
| 109 | 1057 |
| 246 | 1189 |
| 282 | 1212 |
| 309 | 1235 |
| 346 | 1250 |
| 409 | 1283 |
| 517 | 1308 |
| 543 | 1331 |
| 571 | 1370 |
| 583 | 1381 |
| 627 | 1449 |
| 645 | 1480 |
| 666 | 1507 |
| 701 | 1569 |
| 751 | 1590 |
| 791 | 1613 |
| 820 | 1651 |
| 841 | 1678 |
| 868 | 2882 |
| 897 | 2932 |
| 932 | 2959 |
| 946 | 2998 |
| 973 | 3015 |
| 1007 | |

TABLE 3G
Selected FT-Raman peak listing of Compound 1
thiamine HCl co-crystal
Raman Shift (cm⁻¹) ± 2 cm⁻¹

627
751
791
820
1480
1651
2932

FIG. 3H shows the Low Frequency (LF) Raman Spectrum of Compound 1 thiamine HCl co-crystal, and Table 3H below provides LF-Raman peaks.

TABLE 3H
Complete LF-Raman peak listing of Compound 1
thiamine HCl co-crystal
Raman Shift (cm⁻¹)

−346
−107
−77
−62
−40
−29

Compound 1 thiamine HCl co-crystal is unsolvated and consistent with a 1:1 co-crystal based on its ¹H NMR data, provided in FIG. 3I.

### Example 4: Preparation of Compound 1 Ferric Chloride Co-Crystal

In a PEEK grinding cup were combined 20.0 mg of Compound 1 and 12.2 mg of ferric chloride (1 molar equivalent). Ten micro liters of HPLC grade water and a stainless-steel ball were added to the cup. The PEEK grinding cup was capped and placed on a Retsch Mill MM200 and milled at 100% power for 20 minutes. The resulting solids were recovered and analyzed.

Compound 1 ferric chloride co-crystal was analyzed by XRPD, DSC, TGA, and NMR spectroscopy. Characterization data are summarized below in Table 4A.

TABLE 4A

| Sample | Analytical | |
|---|---|---|
| Information | Technique | Results |
| Compound 1 ferric | XRPD | Ferric Chloride Co-crystal |
| chloride co-crystal | DSC | Endo 40.0° C. |
| | TG | 7.7% start to 125° C. |
| | NMR | Consistent with structure |

Compound 1 ferric chloride co-crystal is crystalline based on XRPD data where the sample shows very sharp and well resolved signals as provided in FIG. 4A, as well as in Tables 4B to 4D below.

TABLE 4B
X-ray powder diffraction data of Compound
1 ferric chloride co-crystal

| | Relative | d spacing |
|---|---|---|
| °2θ | Intensity | value (Å) |
| 7.8 | 33 | 11.3633 |
| 11.2 | 20 | 7.9140 |
| 13.5 | 15 | 6.5782 |
| 15.7 | 57 | 5.6515 |
| 16.0 | 19 | 5.5323 |
| 16.2 | 21 | 5.4847 |
| 17.5 | 23 | 5.0619 |
| 17.9 | 14 | 4.9663 |
| 20.6 | 36 | 4.3115 |
| 21.0 | 43 | 4.2302 |
| 22.1 | 49 | 4.0149 |
| 22.4 | 20 | 3.9654 |
| 24.2 | 20 | 3.6806 |
| 24.8 | 23 | 3.5957 |
| 26.2 | 100 | 3.3987 |
| 27.8 | 21 | 3.2136 |
| 28.3 | 50 | 3.1578 |
| 29.4 | 39 | 3.0420 |
| 31.1 | 15 | 2.8774 |
| 31.4 | 18 | 2.8524 |
| 31.6 | 39 | 2.8295 |
| 32.0 | 27 | 2.8002 |
| 32.4 | 27 | 2.7615 |
| 32.6 | 39 | 2.7467 |
| 33.4 | 31 | 2.6843 |
| 33.9 | 24 | 2.6458 |
| 34.2 | 20 | 2.6188 |
| 35.2 | 18 | 2.5481 |
| 36.1 | 26 | 2.4907 |
| 37.6 | 18 | 2.3946 |
| 38.0 | 20 | 2.3667 |
| 38.7 | 22 | 2.3255 |
| 39.7 | 19 | 2.2714 |

TABLE 4C
X-ray powder diffraction selected peak listing and relative
intensities of Compound 1 ferric chloride co-crystal

| | Relative |
|---|---|
| °2θ ± 0.2 °2θ | Intensity |
| 7.8 | 33 |
| 11.2 | 20 |
| 13.5 | 15 |
| 15.7 | 57 |
| 16.0 | 19 |
| 16.2 | 21 |

TABLE 4D
X-ray powder diffraction unique peak and relative
intensity of Compound 1 ferric chloride co-crystal

| | Relative |
|---|---|
| °2θ ± 0.2 °2θ | Intensity |
| 7.8 | 33 |
| 13.5 | 15 |
| 15.7 | 57 |

Compound 1 ferric chloride co-crystal is unsolvated and consistent with the chemical structure based on its ¹H NMR data.

### Example 5: Preparation of Compound 1 Manganese Chloride Co-Crystal

In an HPLC vial were combined 19.5 mg of Compound 1 and 9.0 mg of manganese chloride (1 molar equivalent). A half milliliter of 95:5 ethanol:water was added to the vial and the resulting slurry was stirred magnetically at room temperature for 7 days. The sample was then centrifuged, the mother liquor decanted, and the solids allowed to air dry prior to analysis. Characterization data are summarized below in Table 5A.

TABLE 5A

| Sample | Analytical | |
|---|---|---|
| Information | Technique | Results |
| Compound 1 | XRPD | Manganese Chloride Co-crystal |
| Manganese Chloride | DSC | Various endos below 200° C. |
| Co-crystal | TG | 8.1% start to 150° C. |
| | NMR | Consistent with structure |

Compound 1 Manganese Chloride co-crystal is crystalline based on XRPD data where the sample shows very sharp and well resolved signals as provided in FIG. 5A, as well as in Tables 5B to 5D below.

TABLE 5B
X-ray powder diffraction data of Compound
1 Manganese Chloride Co-crystal

| | Relative | d-spacing |
|---|---|---|
| °2θ | Intensity | value (Å) |
| 10.6 | 13 | 8.3144 |
| 11.4 | 13 | 7.7754 |
| 12.7 | 20 | 6.9920 |
| 14.7 | 17 | 6.0178 |
| 15.3 | 16 | 5.7834 |
| 15.8 | 15 | 5.6159 |
| 17.5 | 48 | 5.0733 |
| 17.8 | 40 | 4.9718 |
| 18.3 | 16 | 4.8584 |
| 19.7 | 92 | 4.5064 |
| 20.5 | 18 | 4.3407 |
| 21.2 | 100 | 4.1830 |
| 21.7 | 58 | 4.0953 |
| 22.7 | 24 | 3.9137 |
| 24.3 | 21 | 3.6627 |
| 24.7 | 52 | 3.6014 |
| 24.9 | 43 | 3.5730 |
| 25.4 | 18 | 3.5065 |
| 26.5 | 32 | 3.3634 |
| 26.8 | 44 | 3.3216 |
| 27.5 | 69 | 3.2480 |
| 28.1 | 21 | 3.1732 |
| 28.8 | 54 | 3.0977 |
| 29.7 | 42 | 3.0079 |
| 30.1 | 37 | 2.9650 |
| 30.9 | 43 | 2.8956 |
| 31.9 | 32 | 2.8036 |
| 32.2 | 21 | 2.7799 |
| 32.8 | 25 | 2.7320 |
| 33.2 | 23 | 2.6952 |
| 33.8 | 35 | 2.6488 |
| 34.4 | 23 | 2.6040 |
| 35.3 | 45 | 2.5439 |
| 35.5 | 36 | 2.5287 |
| 37.7 | 21 | 2.3872 |
| 38.8 | 26 | 2.3220 |
| 39.6 | 51 | 2.2736 |

TABLE 5C
X-ray powder diffraction selected peak listing and relative
intensities of Compound 1 Manganese Chloride Co-crystal

| | Relative |
|---|---|
| °2θ ± 0.2 °2θ | Intensity |
| 10.6 | 13 |
| 11.4 | 13 |
| 12.7 | 20 |
| 14.7 | 17 |
| 15.3 | 16 |
| 15.8 | 15 |

TABLE 5D
X-ray powder diffraction unique peak and relative intensity
of Compound 1 Manganese chloride co-crystal

| | Relative |
|---|---|
| °2θ ± 0.2 °2θ | Intensity |
| 7.8 | 33 |
| 13.5 | 15 |
| 15.7 | 57 |

Compound 1 Manganese Chloride co-crystal is unsolvated and consistent with the chemical structure based on its ¹H NMR data.

### Example 6: Preparation of Compound 1 Zinc Chloride Co-Crystal

In a PEEK grinding cup were combined 19.8 mg of Compound 1 and 10.1 mg of zinc chloride (1 molar equivalent). Ten micro liters of water and a stainless-steel ball were added to the cup. The PEEK grinding cup was capped and placed on a Retsch Mill MM200 and milled at 100% power for 20 minutes. The resulting solids were recovered and analyzed. Characterization data are summarized below in Table 6A.

TABLE 6A

| Sample | Analytical | |
|---|---|---|
| Information | Technique | Results |
| Compound 1 Zinc | XRPD | Zinc Chloride Co-crystal |
| Chloride Co-crystal | DSC | Endo 124.1, 153.0° C. |
| | TG | 3.6% start to 150° C. |
| | NMR | Consistent with structure |
| | | Two small impurities (2.7, 2.9 ppm) |

Compound 1 Zinc Chloride co-crystal is crystalline based on XRPD data where the sample shows very sharp and well resolved signals.

TABLE 6B
X-ray powder diffraction data of Compound
1 Zinc Chloride Co-crystal

| | Relative | d spacing |
|---|---|---|
| °2θ | Intensity | value (Å) |
| 11.2 | 51 | 7.8999 |
| 13.4 | 29 | 6.6075 |
| 15.1 | 16 | 5.8518 |
| 16.5 | 20 | 5.3659 |
| 17.1 | 18 | 5.1973 |
| 17.5 | 19 | 5.0791 |
| 18.3 | 63 | 4.8531 |
| 18.7 | 35 | 4.7551 |
| 19.3 | 69 | 4.6083 |
| 20.7 | 31 | 4.2950 |
| 21.4 | 45 | 4.1521 |
| 22.0 | 19 | 4.0438 |
| 22.1 | 35 | 4.0149 |
| 22.5 | 88 | 3.9584 |
| 22.8 | 37 | 3.9036 |
| 23.0 | 16 | 3.8601 |
| 23.4 | 31 | 3.8079 |
| 24.0 | 52 | 3.7139 |
| 24.9 | 21 | 3.5815 |
| 25.1 | 35 | 3.5506 |
| 26.9 | 100 | 3.3192 |
| 27.2 | 36 | 3.2808 |
| 28.1 | 44 | 3.1755 |
| 28.4 | 52 | 3.1448 |
| 29.0 | 20 | 3.0768 |
| 29.3 | 16 | 3.0522 |
| 29.6 | 52 | 3.0219 |
| 30.1 | 49 | 2.9650 |
| 30.5 | 28 | 2.9271 |
| 31.4 | 22 | 2.8453 |
| 31.7 | 19 | 2.8226 |
| 31.9 | 21 | 2.8036 |
| 32.4 | 35 | 2.7615 |
| 32.8 | 73 | 2.7288 |
| 33.3 | 31 | 2.6874 |
| 34.4 | 20 | 2.6084 |
| 35.1 | 29 | 2.5537 |
| 35.6 | 20 | 2.5204 |
| 36.0 | 16 | 2.4920 |
| 36.8 | 23 | 2.4423 |
| 37.0 | 19 | 2.4283 |
| 37.8 | 20 | 2.3787 |
| 38.6 | 21 | 2.3313 |

TABLE 6C
X-ray powder diffraction selected peak listing and relative
intensities of Compound 1 Zinc Chloride Co-crystal

| | Relative |
|---|---|
| °2θ ± 0.2 °2θ | Intensity |
| 11.2 | 51 |
| 13.4 | 29 |
| 15.1 | 16 |
| 16.5 | 20 |
| 17.1 | 18 |

TABLE 6D
X-ray powder diffraction unique peak and relative
intensity of Compound 1 Zinc chloride co-crystal

| | Relative |
|---|---|
| °2θ ± 0.2 °2θ | Intensity |
| 13.4 | 29 |
| 15.1 | 16 |
| 17.1 | 18 |

Compound 1 Zinc Chloride co-crystal is unsolvated and consistent with the chemical structure though two small impurities at 2.7 and 2.9 ppm were observed in its ¹H NMR data.

1. A co-crystal comprising:
Compound I 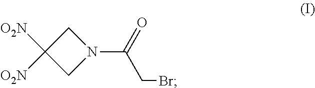 [SMILES: O=C(CBr)N1CC([N+](=O)[O-])([N+](=O)[O-])C1]
and
calcium chloride, pyridoxine HCl, thiamine HCl, ferric chloride, manganese chloride, or zinc chloride.

2. The co-crystal of claim 1, wherein the co-crystal comprises Compound I and calcium chloride and is characterized by an XRPD pattern comprising peaks at angles 2-theta of 10.8±0.2, 11.6±0.2, and 14.9±0.2 degrees.

3.-19. (canceled)

20. The co-crystal of claim 1, wherein the co-crystal comprises Compound I and pyridoxine HCl and is characterized by an XRPD pattern comprising peaks at angles 2-theta of 11.4±0.2, 12.7±0.2, and 14.8±0.2 degrees.

21.-37. (canceled)

38. The co-crystal of claim 1, wherein the co-crystal comprises Compound I and thiamine HCl and is characterized by an XRPD pattern comprising peaks at angles 2-theta of 8.2±0.2, 10.3±0.2, and 16.4±0.2 degrees.

39.-55. (canceled)

56. The co-crystal of claim 1, wherein the co-crystal comprises Compound I and ferric chloride and is characterized by an XRPD pattern comprising peaks at angles 2-theta of 7.8±0.2, 13.5±0.2, and 15.7±0.2 degrees.

57.-65. (canceled)

66. The co-crystal of claim 1, wherein the co-crystal comprises Compound I and manganese chloride and is characterized by an XRPD pattern comprising peaks at angles 2-theta of 12.7±0.2, 14.7±0.2, and 15.3±0.2 degrees.

67-75. (canceled)

76. The co-crystal of claim 1, wherein the co-crystal comprises Compound I and zinc chloride and is characterized by an XRPD pattern comprising peaks at angles 2-theta of 13.4±0.2, 15.1±0.2, and 17.1±0.2 degrees.

77.-84. (canceled)

85. A pharmaceutical composition comprising a co-crystal of claim 1 and a pharmaceutically acceptable excipient.

86. The pharmaceutical composition of claim 85, further comprising: a blood product.

87. The pharmaceutical composition of claim 86, wherein the blood product:
comprises erythrocyte cells;
is a mixture of packed red blood cells; or
is whole blood.

88.-91. (canceled)

92. The pharmaceutical composition of claim 85, further comprising: at least one agent.

93. The pharmaceutical composition of claim 92, wherein each agent of the at least one agent is selected from the group consisting of:
a sympathomimetic drug, an anti-inflammatory agent, an anti-diabetic agent, an anti-fibrotic agent, an anti-steatotic agent, a cholesterol/lipid modulating agent, an anti-cancer agent, and an anti-diabetic agent; or,
an antioxidant, a vitamin, a mineral, a steroid, a growth hormone, a nitric oxide donor, a terpene-indole alkaloid compound, a bisphosphonate compound, a glucocorticoid, a coagulation factor, a narcotic, an opioid receptor agonist, a narcotic receptor antagonist, an anti-viral agent, a biologic response modifier (BRM) agent, and an antigen.

94. (canceled)

95. A method for preventing or treating a disease, a disorder, a condition, or an infection, in a subject in need thereof, the method comprising: administering an effective amount of the co-crystal of claim 1 to the subject.

96.-99. (canceled)

100. The method of claim 95, wherein the disease is:
associated with abnormal cell proliferation;
selected from the group consisting of a neurodegenerative disease, an allergic disease, an autoimmune disease, a fibrotic disease, an inflammatory disease, an infectious disease, a pulmonary disease, a reproductive-related disease, a cardiovascular disease, and a metabolic disease; or
cancer.

101.-107. (canceled)

108. The method of claim 95, wherein the disease, the disorder, or the condition is selected from the group consisting of non-alcoholic fatty liver disease (NAFLD), non-alcoholic steatohepatitis (NASH), atherosclerosis, hyperlipidemia, hypercholesterolemia, steatosis, liver fibrosis, coronary heart disease, peripheral vascular disease, stroke, aortic aneurysm, glucose intolerance, diabetes, muscular dystrophies, patent foramen ovale, obesity, mitochondrial disorders or diseases, chronic obstructive pulmonary disease, hyperCKemia, motor neuron disease, neuromuscular disease, multiple sclerosis, Charcot-myositis including polymyositis and dermatomyositis, insulin resistance, myoedema, rhabdomyolysis, idiopathic chronic muscle fatigue, reduced skeletal muscle function, and disrupted skeletal muscle function or metabolism.

109.-117. (canceled)

118. The method of claim 95, wherein the infection comprises a bacterial infection and wherein the condition comprises a hemolytic, ischemic, or hypoxic condition.

119.-127. (canceled)

128. A method of protecting a subject in need thereof against normal tissue toxicity caused by chemotherapy and/or radiation therapy, the method comprising: subcutaneously administering to the subject an effective amount of the co-crystal of claim 1 before the subject is exposed to the chemotherapy and/or the radiation therapy.

129. A method of treating or preventing neurodegenerative, allergic, autoimmune, fibrotic, inflammatory, infectious, pulmonary, cardiac, vascular, or metabolic diseases in a subject in need thereof, the method comprising: subcutaneously administering to the subject in an effective amount of the co-crystal of claim 1.

130. A method of treating a subject suffering from reduced blood volume or low perfusion, the method comprising: administering to the subject a blood product comprising the co-crystal of claim 1.

131. A method of preparing the co-crystal of claim 1, the method comprising:
wherein the co-crystal comprises Compound I and calcium chloride:
a) mixing molar equivalents of Compound I and calcium chloride;
b) mixing ethanol and water with Compound I and calcium chloride to form a slurry;
c) stirring the slurry of step b) to obtain stirred slurry;
d) filtering the stirred slurry of step c) to obtain solid content; and
e) drying the solid content of step d); or,
wherein the co-crystal comprises Compound I and pyridoxine HCl:
a) mixing molar equivalents of Compound I and pyridoxine HCl;
b) mixing ethanol and water with Compound I and pyridoxine HCl to form a slurry;
c) stirring the slurry of step b) to obtain stirred slurry;
d) filtering the stirred slurry of step c) to obtain solid content; and
e) drying the solid content of step d); or,
wherein the co-crystal comprises Compound I and thiamine HCl:
a) mixing molar equivalents of Compound I and thiamine HCl;
b) mixing ethanol and water with Compound I and thiamine HCl to form a slurry;
c) stirring the slurry of step b) to obtain stirred slurry;
d) filtering the stirred slurry of step c) to obtain solid content; and
e) drying the solid content of step d); or
wherein the co-crystal comprises Compound I and ferric chloride:
a) mixing molar equivalents of Compound I and ferric chloride;
b) mixing ethanol and water with Compound I and ferric chloride to form a slurry;
c) stirring the slurry of step b) to obtain stirred slurry;
d) filtering the stirred slurry of step c) to obtain solid content; and
e)drying the solid content of step d); or,
wherein the co-crystal comprises Compound I and manganese chloride:
a) mixing molar equivalents of Compound I and manganese chloride;
b) mixing ethanol and water with Compound I and manganese chloride to form a slurry;
c) stirring the slurry of step b) to obtain stirred slurry;
d) filtering the stirred slurry of step c) to obtain solid content; and
e) driving the solid content of step d); or,
wherein the co-crystal comprises Compound I and zinc chloride:
a) mixing molar equivalents of Compound I and zinc chloride;
b) mixing ethanol and water with Compound I and zinc chloride to form a slurry;
c) stirring the slurry of step b) to obtain stirred slurry;
d) filtering the stirred slurry of step c) to obtain solid content; and
e) driving the solid content of step d).

132.-136. (canceled)
